SEQUENCE LISTING (76 sequences)

SEQ ID NO: 1 - protein, 262 aa, Artificial Sequence
  Description of Artificial Sequence Synthetic polypeptide
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKAL

SEQ ID NO: 2 - DNA, 786 bp, Artificial Sequence
  Description of Artificial Sequence Synthetic polynucleotide
atggactggatctggcgcatcctgtttctcgtgggagccgccacaggcgcccattctgaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctcta

SEQ ID NO: 3 - protein, 415 aa, Artificial Sequence
  Description of Artificial Sequence Synthetic polypeptide
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - DNA, 1245 bp, Artificial Sequence
  Description of Artificial Sequence Synthetic polynucleotide
atggactggatctggcgcatcctgtttctcgtgggagccgccacaggcgcccattctgaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctctagatctcgagcccaaatcttgtgacaaaactcacacatgcccaccgtgcccggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 5 - protein, 462 aa, Artificial Sequence
  Description of Artificial Sequence Synthetic polypeptide
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - DNA, 1386 bp, Artificial Sequence
  Description of Artificial Sequence Synthetic polynucleotide
atggactggatctggcgcatcctgtttctcgtgggagccgccacaggcgcccattctgaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctctagatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 7 - protein, 267 aa, Artificial Sequence
  Description of Artificial Sequence Synthetic polypeptide
MDWIWRILFLVGAATGAHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIK

SEQ ID NO: 8 - DNA, 801 bp, Artificial Sequence
  Description of Artificial Sequence Synthetic polynucleotide
atggactggatctggcgcatcctgtttctcgtgggagccgccacaggcgcccattctcaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaag

SEQ ID NO: 9 - protein, 467 aa, Artificial Sequence
  Description of Artificial Sequence Synthetic polypeptide
MDWIWRILFLVGAATGAHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - DNA, 1401 bp, Artificial Sequence
  Description of Artificial Sequence Synthetic polynucleotide
atggactggatctggcgcatcctgtttctcgtgggagccgccacaggcgcccattctcaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaaggatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 11 - protein, 323 aa, Artificial Sequence
  Description of Artificial Sequence Synthetic polypeptide
MDWIWRILFLVGAATGAHSQVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIKDLEPKSCDKTHTCPPCPDPKFWVLVVVGGVLACYSLLVTVAFIIFWVRSKRSRLLH

SEQ ID NO: 12 - DNA, 969 bp, Artificial Sequence
  Description of Artificial Sequence Synthetic polynucleotide
atggactggatctggcgcatcctgtttctcgtgggagccgccacaggcgcccattctcaggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaaggatctcgagcccaaatcttgtgacaaaactcacacatgcccaccgtgcccggatcccaaattttgggtgctggtggtggttggtggagtcctggcttgctatagcttgctagtaacagtggcctttattattttctgggtgaggagtaagaggagcaggctcctgcac

SEQ ID NO: 13 - protein, 810 aa, Artificial Sequence
  Description of Artificial Sequence Synthetic polypeptide
MDWIWRILFLVGAATGAHSQVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIKDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRASRAEGRGSLLTCGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 14 - DNA, 2433 bp, Artificial Sequence
  Description of Artificial Sequence Synthetic polynucleotide
atggactggatctggcggattctgttcctcgtgggagccgccacaggcgctcactcacaggtgcagctgctggaatctggcggcggactggtgcagcctggcggcagcctgagactgagctgcgccgccagcggcttcaccttcagcagctacaccatgagctgggtccggcaggctcctggacaggccctggaatggatgggcaccatcagcagcggcggcacctacacctactaccccgacagcgtgaagggccggttcaccatcagccgggacaacgccaagaacagcctgtacctgcagatgaacagcctgagagccgaggacacagccgtgtactactgcgccagagaggccatcttcacctactggggcagaggcaccctggtcacaagcagcggaggcggaggaagtggagggggaggatcaggcggcggaggcagcgatatccagctgacccagagccctagcagcctgagcgccagcgtgggcgacagagtgaccatcacatgcaaggccagccaggacatcaacaactacctgagctggtatcagcagaagcccggccaggcccccagactgctgatctaccgggccaacagactggtggacggcgtgcccgatagattcagcggcagcggctacggcaccgacttcaccctgaccatcaacaacatcgagtccgaggacgccgcctactacttctgcctgaagtacgacgtgttcccctacaccttcggccagggcaccaaggtggagatcaaggatctcgagcccaaatcttgtgacaaaactcacacatgcccaccgtgcccggatcccaagttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaagagcctctagagccgagggcagaggcagcctgctgacatgtggcgacgtggaagagaacccaggccccatgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttctaa

SEQ ID NO: 15 - protein, 19 aa, Artificial Sequence
  Description of Artificial Sequence Synthetic peptide
MDWIWRILFLVGAATGAHS

SEQ ID NO: 16 - DNA, 57 bp, Artificial Sequence
  Description of Artificial Sequence Synthetic oligonucleotide
atggactggatctggcgcatcctgtttctcgtgggagccgccacaggcgcccattct

SEQ ID NO: 17 - protein, 243 aa, Artificial Sequence
  Description of Artificial Sequence Synthetic polypeptide
EVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKAL

SEQ ID NO: 18 - DNA, 729 bp, Artificial Sequence
  Description of Artificial Sequence Synthetic polynucleotide
gaggtacaactgcagcagtctggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcaaagctcta

SEQ ID NO: 19 - protein, 17 aa, Artificial Sequence
  Description of Artificial Sequence Synthetic peptide
DLEPKSCDKTHTCPPCP

SEQ ID NO: 20 - DNA, 51 bp, Artificial Sequence
  Description of Artificial Sequence Synthetic oligonucleotide
gatctcgagcccaaatcttgtgacaaaactcacacatgcccaccgtgcccg

SEQ ID NO: 21 - protein, 20 aa, Artificial Sequence
  Description of Artificial Sequence Synthetic peptide
DLEPKSCDKTHTCPPCPDPK

SEQ ID NO: 22 - DNA, 60 bp, Artificial Sequence
  Description of Artificial Sequence Synthetic oligonucleotide
gatctcgagcccaaatcttgtgacaaaactcacacatgcccaccgtgcccggatcccaaa

SEQ ID NO: 23 - protein, 21 aa, Artificial Sequence
  Description of Artificial Sequence Synthetic peptide
LCYLLDGILFIYGVILTALFL

SEQ ID NO: 24 - DNA, 63 bp, Artificial Sequence
  Description of Artificial Sequence Synthetic oligonucleotide
ctctgctacctgctggatggaatcctcttcatctatggtgtcattctcactgccttgttcctg

SEQ ID NO: 25 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - DNA, 336 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 27 - protein, 133 aa, Artificial Sequence
  Description of Artificial Sequence Synthetic polypeptide
LCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - DNA, 399 bp, Artificial Sequence
  Description of Artificial Sequence Synthetic polynucleotide
ctctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 29 - protein, 248 aa, Artificial Sequence
  Description of Artificial Sequence Synthetic polypeptide
QVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAKTTPPKLEEGEFSEARVDIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIK

SEQ ID NO: 30 - DNA, 744 bp, Artificial Sequence
  Description of Artificial Sequence Synthetic polynucleotide
caggtgcagctgcagcagcctggcgctgaactcgtgcggccaggcgcttctgtgaagctgagctgtaaagccagcggctacaccttcagcaactacctgatgaactgggtcaagcagcggcccgagcaggacctggattggatcggcagaatcgacccctacgacggcgacatcgactacaaccagaacttcaaggacaaggccatcctgaccgtggacaagagcagcagcaccgcctacatgcagctgtccagcctgaccagcgaggacagcgccgtgtactactgcgccagaggctacggcacagcctacggcgtggactattggggccagggcacaagcgtgaccgtgtccagcgccaagaccaccccccctaagctggaagagggcgagttctccgaggcccgggtggacattgtgctgacacagtctccagccagcctggccgtgtccctgggacagagagccaccatcagctgtagggccagcgagagcgtggacaactacggcatcagcttcatgaattggttccagcagaagcccggccagccccccaagctgctgatctatgccgccagcagacagggcagcggagtgcctgccagattttctggcagcggctccggcaccgacttcagcctgaacatccaccctatggaagaggacgacaccgccatgtacttttgccagcagagcaaagaggtgccctggacctttggcggaggcaccaagctggaaatcaag

SEQ ID NO: 31 - protein, 27 aa, Artificial Sequence
  Description of Artificial Sequence Synthetic peptide
FWVLVVVGGVLACYSLLVTVAFIIFWV

SEQ ID NO: 32 - DNA, 81 bp, Artificial Sequence
  Description of Artificial Sequence Synthetic oligonucleotide
ttttgggtgctggtggtggttggtggagtcctggcttgctatagcttgctagtaacagtggcctttattattttctgggtg

SEQ ID NO: 33 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 34 - DNA, 123 bp, Artificial Sequence
  Description of Artificial Sequence Synthetic polynucleotide
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 35 - protein, 68 aa, Artificial Sequence
  Description of Artificial Sequence Synthetic polypeptide
FWVLVVVGGVLACYSLLVTVAFIIFWVRSKRSRLLHSDYMNMTPRRPGPTRKHYQPYAPPRDFAAYRS

SEQ ID NO: 36 - DNA, 204 bp, Artificial Sequence
  Description of Artificial Sequence Synthetic polynucleotide
ttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcc

SEQ ID NO: 37 - DNA, 57 bp, Artificial Sequence
  Description of Artificial Sequence Synthetic oligonucleotide
atggactggatctggcggattctgttcctcgtgggagccgccacaggcgctcactca

SEQ ID NO: 38 - protein, 236 aa, Artificial Sequence
  Description of Artificial Sequence Synthetic polypeptide
QVQLLESGGGLVQPGGSLRLSCAASGFTFSSYTMSWVRQAPGQALEWMGTISSGGTYTYYPDSVKGRFTISRDNAKNSLYLQMNSLRAEDTAVYYCAREAIFTYWGRGTLVTSSGGGGSGGGGSGGGGSDIQLTQSPSSLSASVGDRVTITCKASQDINNYLSWYQQKPGQAPRLLIYRANRLVDGVPDRFSGSGYGTDFTLTINNIESEDAAYYFCLKYDVFPYTFGQGTKVEIK

SEQ ID NO: 39 - DNA, 708 bp, Artificial Sequence
  Description of Artificial Sequence Synthetic polynucleotide
caggtgcagctgctggaatctggcggcggactggtgcagcctggcggcagcctgagactgagctgcgccgccagcggcttcaccttcagcagctacaccatgagctgggtccggcaggctcctggacaggccctggaatggatgggcaccatcagcagcggcggcacctacacctactaccccgacagcgtgaagggccggttcaccatcagccgggacaacgccaagaacagcctgtacctgcagatgaacagcctgagagccgaggacacagccgtgtactactgcgccagagaggccatcttcacctactggggcagaggcaccctggtcacaagcagcggaggcggaggaagtggagggggaggatcaggcggcggaggcagcgatatccagctgacccagagccctagcagcctgagcgccagcgtgggcgacagagtgaccatcacatgcaaggccagccaggacatcaacaactacctgagctggtatcagcagaagcccggccaggcccccagactgctgatctaccgggccaacagactggtggacggcgtgcccgatagattcagcggcagcggctacggcaccgacttcaccctgaccatcaacaacatcgagtccgaggacgccgcctactacttctgcctgaagtacgacgtgttcccctacaccttcggccagggcaccaaggtggagatcaag

SEQ ID NO: 40 - protein, 15 aa, Artificial Sequence
  Description of Artificial Sequence Synthetic peptide
EPKSCDKTHTCPPCP

SEQ ID NO: 41 - DNA, 45 bp, Artificial Sequence
  Description of Artificial Sequence Synthetic oligonucleotide
gagcccaaatcttgtgacaaaactcacacatgcccaccgtgcccg

SEQ ID NO: 42 - DNA, 60 bp, Artificial Sequence
  Description of Artificial Sequence Synthetic oligonucleotide
gatctcgagcccaaatcttgtgacaaaactcacacatgcccaccgtgcccggatcccaag

SEQ ID NO: 43 - DNA, 204 bp, Artificial Sequence
  Description of Artificial Sequence Synthetic polynucleotide
ttctgggtgctggtggtcgtgggcggagtgctggcctgttacagcctgctcgtgaccgtggccttcatcatcttttgggtgcgcagcaagcggagccggctgctgcacagcgactacatgaacatgacccccagacggcctggccccaccagaaagcactaccagccttacgcccctcccagagacttcgccgcctaccggtcc

SEQ ID NO: 44 - DNA, 336 bp, Artificial Sequence
  Description of Artificial Sequence Synthetic polynucleotide
agagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagaagaaagaacccccaggaaggcctgtataacgaactgcagaaagacaagatggccgaggcctacagcgagatcggcatgaagggcgagcggagaagaggcaagggccacgatggcctgtaccagggactgagcaccgccaccaaggacacctacgacgccctgcacatgcaggccctgcctccaaga

SEQ ID NO: 45 - protein, 18 aa, Thosea asigna virus
EGRGSLLTCGDVEENPGP

SEQ ID NO: 46 - DNA, 54 bp, Thosea asigna virus
gagggcagaggcagcctgctgacatgtggcgacgtggaagagaacccaggcccc

SEQ ID NO: 47 - protein, 333 aa, Artificial Sequence
  Description of Artificial Sequence Synthetic polypeptide
MPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 48 - DNA, 999 bp, Artificial Sequence
  Description of Artificial Sequence Synthetic polynucleotide
atgcctccccccagactgctgttcttcctgctgttcctgacccctatggaagtgcggcccgaggaacccctggtcgtgaaagtggaagagggcgacaacgccgtgctgcagtgtctgaagggcacctccgatggccctacccagcagctgacctggtccagagagagccccctgaagcccttcctgaagctgtctctgggcctgcctggcctgggcatccatatgaggccactggccatctggctgttcatcttcaacgtgtcccagcagatgggaggcttctacctgtgccagcctggcccaccttctgagaaggcttggcagcctggctggaccgtgaacgtggaaggatctggcgagctgttccggtggaacgtgtccgatctgggcggcctgggatgcggcctgaagaacagatctagcgagggccccagcagccccagcggcaaactgatgagccccaagctgtacgtgtgggccaaggacagacccgagatttgggagggcgagcccccttgcctgccccctagagatagcctgaaccagagcctgagccaggacctgacaatggcccctggcagcacactgtggctgagctgtggcgtgccacccgactctgtgtctagaggccctctgagctggacccacgtgcaccctaagggccctaagagcctgctgtccctggaactgaaggacgacaggcccgccagagatatgtgggtcatggaaaccggcctgctgctgcctagagccacagcccaggatgccggcaagtactactgccacagaggcaacctgaccatgagcttccacctggaaatcaccgccagacccgtgctgtggcactggctgctgagaaccggcggatggaaagtgtccgccgtgactctggcctacctgatcttctgcctgtgctccctcgtgggcatcctgcatctgcagagggctctggtgctgcggcggaagcggaagagaatgaccgaccctacccggcggttc

SEQ ID NO: 49 - protein, 30 aa, Artificial Sequence
  Description of Artificial Sequence Synthetic polypeptide
CDIYIWAPLAGTCGVLLLSLVITLYCNHRN

SEQ ID NO: 50 - DNA, 90 bp, Artificial Sequence
  Description of Artificial Sequence Synthetic oligonucleotide
tgcgacatctacatctgggcccctctggccggcacatgtggcgtgctgctgctgagcctcgtgatcaccctgtactgcaaccaccggaac

SEQ ID NO: 51 - protein, 31 aa, Artificial Sequence
  Description of Artificial Sequence Synthetic polypeptide
QPMALIVLGGVAGLLLFIGLGIFFCVRCRHR

SEQ ID NO: 52 - DNA, 93 bp, Artificial Sequence
  Description of Artificial Sequence Synthetic oligonucleotide
cagccaatggccctgattgtgctggggggcgtcgccggcctcctgcttttcattgggctaggcatcttcttctgtgtcaggtgccggcaccga

SEQ ID NO: 53 - protein, 21 aa, Artificial Sequence
  Description of Artificial Sequence Synthetic peptide
GSGEGRGSLLTCGDVEENPGP

SEQ ID NO: 54 - protein, 63 aa, Artificial Sequence
  Description of Artificial Sequence Synthetic polypeptide
GSGSRVTELLYRMKRAETYCPRPLLAIHPTEARHKQKIVAPVKQLLNFDLLKLAGDVESNPGP

SEQ ID NO: 55 - protein, 19 aa, Amphimedon queenslandica
LLCFLLLLLSGDVELNPGP

SEQ ID NO: 56 - protein, 20 aa, Amphimedon queenslandica
HHFMFLLLLLAGDIELNPGP

SEQ ID NO: 57 - protein, 20 aa, Saccoglossus kowalevskii
WFLVLLSFILSGDIEVNPGP

SEQ ID NO: 58 - protein, 20 aa, Branchiostoma floridae
KNCAMYMLLLSGDVETNPGP

SEQ ID NO: 59 - protein, 20 aa, Branchiostoma floridae
MVISQLMLKLAGDVEENPGP

SEQ ID NO: 60 - protein, 22 aa, Artificial Sequence
  Description of Artificial Sequence Synthetic peptide
GSGATNFSLLKQAGDVEENPGP

SEQ ID NO: 61 - protein, 23 aa, Artificial Sequence
  Description of Artificial Sequence Synthetic peptide
GSGQCTNYALLKLAGDVESNPGP

SEQ ID NO: 62 - protein, 8 aa, Unknown
  Description of Unknown 2A consensus sequence
  MOD_RES (2)..(2): Any amino acid
  MOD_RES (4)..(4): Any amino acid
DXEXNPGP

SEQ ID NO: 63 - DNA, 23 bp, Artificial Sequence
  Description of Artificial Sequence Synthetic oligonucleotide
  modified_base (21)..(21): a, c, t, or g
  misc_feature (21)..(21): n is a, c, g, or t
cctgcatgatgcgcggcccangg

SEQ ID NO: 64 - DNA, 23 bp, Artificial Sequence
  Description of Artificial Sequence Synthetic oligonucleotide
  modified_base (21)..(21): a, c, t, or g
  misc_feature (21)..(21): n is a, c, g, or t
caagtagtcggggatgtcggngg

SEQ ID NO: 65 - DNA, 23 bp, Artificial Sequence
  Description of Artificial Sequence Synthetic oligonucleotide
  modified_base (21)..(21): a, c, t, or g
  misc_feature (21)..(21): n is a, c, g, or t
agtagtcggggatgtcggcgngg

SEQ ID NO: 66 - protein, 9 aa, Artificial Sequence
  Description of Artificial Sequence Synthetic peptide
RSKRSRLLH

SEQ ID NO: 67 - DNA, 27 bp, Artificial Sequence
  Description of Artificial Sequence Synthetic oligonucleotide
aggagtaagaggagcaggctcctgcac

SEQ ID NO: 68 - DNA, 23 bp, Artificial Sequence
  Description of Artificial Sequence Synthetic oligonucleotide
  modified_base (21)..(21): a, c, t, or g
  misc_feature (21)..(21): n is a, c, g, or t
gcatgatgcgcggcccaaggngg

SEQ ID NO: 69 - DNA, 57 bp, Artificial Sequence
  Description of Artificial Sequence Synthetic primer
cactctttccctacacgacgctcttccgatcttcccgatgaccctgtcttcccgtgc

SEQ ID NO: 70 - DNA, 59 bp, Artificial Sequence
  Description of Artificial Sequence Synthetic primer
gtgactggagttcagacgtgtgctcttccgatctaggtagaagccattagtgagctggc

SEQ ID NO: 71 - DNA, 25 bp, Artificial Sequence
  Description of Artificial Sequence Synthetic oligonucleotide
tcccgatgaccctgtcttcccgtgc

SEQ ID NO: 72 - DNA, 25 bp, Artificial Sequence
  Description of Artificial Sequence Synthetic oligonucleotide
aggtagaagccattagtgagctggc

SEQ ID NO: 73 - DNA, 26 bp, Artificial Sequence
  Description of Artificial Sequence Synthetic oligonucleotide
ggtcccatcactgttgaatcctctgt

SEQ ID NO: 74 - DNA, 25 bp, Artificial Sequence
  Description of Artificial Sequence Synthetic oligonucleotide
accctctggctgctggatgtgctga

SEQ ID NO: 75 - DNA, 20 bp, Artificial Sequence
  Description of Artificial Sequence Synthetic oligonucleotide
cctgcatgatgcgcggccca

SEQ ID NO: 76 - DNA, 20 bp, Artificial Sequence
  Description of Artificial Sequence Synthetic oligonucleotide
gcatgatgcgcggcccaagg

(19) United States
(12) Patent Application Publication
(10) Pub. No.: US 2024/0366759 A1
(43) Pub. Date: Nov. 7, 2024

(54) METHOD FOR PRESERVING DEVELOPMENTAL POTENTIAL OF IMMUNE CELLS USED FOR ADOPTIVE CELLULAR THERAPIES

(71) Applicant: St. Jude Children's Research Hospital, Inc., Memphis, TN (US)

(72) Inventors: Benjamin YOUNGBLOOD, Memphis, TN (US); Giedre KRENCIUTE, Memphis, TN (US); Stephen GOTTSCHALK, Germantown, TN (US); Brooke PRINZING, Memphis, TN (US); Caitlin ZEBLEY, Memphis, TN (US)

(73) Assignee: St. Jude Children's Research Hospital, Inc., Memphis, TN (US)

(21) Appl. No.: 18/572,990

(22) PCT Filed: Jun. 21, 2022
(86) PCT No.: PCT/US2022/034257

§ 371 Date: Dec. 21, 2023

(51) Int. Cl. A61K 39/00; C12N 5/0783; C12N 9/22; C12N 15/11; C12N 15/90

(52) U.S. Cl. CPC A61K 39/4611; A61K 39/4631; A61K 39/4635; C12N 5/0636; C12N 9/22; C12N 15/11; C12N 15/907; C12N 2310/20; C12N 2501/231; C12N 2501/2315; C12N 2502/30

(57) ABSTRACT

The application provides modified immune effector cells wherein the DNA (cytosine-5)-methyltransferase 3A (DNMT3A)-mediated de novo DNA methylation of the cell genome is inhibited, and IL10 signaling pathway is enhanced. The application also provides related pharmaceutical compositions and the methods for generating such modified immune effector cells. The application further provides uses of such modified immune effector cells for treating diseases such as cancers, infectious diseases and autoimmune diseases.

### CROSS REFERENCE TO RELATED APPLICATIONS

This application claims priority to U.S. Provisional Application No. 63/213,503, filed Jun. 22, 2021, the disclosure of which is herein incorporated by reference in its entirety.

### STATEMENT REGARDING FEDERALLY SPONSORED RESEARCH

This invention was made with government support under grant number CA237311 awarded by the National Institutes of Health. The government has certain rights in the invention.

### FIELD OF THE INVENTION

The application relates to modified immune effector cells with preserved developmental potential, as well as related pharmaceutical compositions. The application further relates to methods for generating the modified immune effector cell and methods for using the modified immune effector cell (e.g., adoptive cell therapy) for treatment of diseases.

### SEQUENCE LISTING

The instant application contains a Sequence Listing which has been submitted electronically in ASCII format and is hereby incorporated by reference in its entirety. Said ASCII copy, created on Jun. 21, 2022, is named 243734_000170_SL.txt and is 74,569 bytes in size.

### BACKGROUND

Cellular immunotherapy with adoptively transferred chimeric antigen receptor (CAR)-modified T cells is an attractive approach to improve outcomes for patients with cancer. However, T cell therapy for solid tumors has shown limited antitumor activity in early phase clinical studies. Even for the most successful CAR T cell therapy (1), CD19-CAR T cell therapy for CD19+ acute lymphoblastic leukemia (ALL), only 50% of patients have responses that last more than one year (11). Complete responses are much lower for CD19+ chronic lymphocytic leukemia (CLL) (12), and only few long-term survivors have been reported for CAR T cell therapies targeting solid tumor or brain tumor antigens such as HER2, mesothelin, CECAM5, GD2, EGFRvIII, and IL13Rα2 (13-18). There exists a need in the art for developing improved antigen-specific T cell therapy. This need can be met with a modified immune effector cell with preserved multipotency, as disclosed herein.

### SUMMARY OF THE INVENTION

As specified in the Background section above, there is a great need in the art for modified immune effector cells with preserved multipotency (i.e., developmental potential) for use in cell therapy for cancer and other disease (e.g., infectious or autoimmune diseases). The present application addresses these and other needs.

In one aspect is provided a modified immune effector cell, wherein DNA (cytosine-5)-methyltransferase 3A (DNMT3A)-mediated de novo DNA methylation of the cell genome may be inhibited, and IL10 signaling pathway may be enhanced. In some embodiments, DNMT3A-mediated methylaytion is inhibited by inhibting the enzymatic activity of the DNMT3A protein in the cell. In some embodiments, the enzymatic activity of the DNMT3A protein may be inhibited by exposing the cell to a DNMT3A active site inhibitor. In some embodiments, the DNMT3A gene may be mutated in a DNMT3A catalytic domain so that the enzymatic activity of the DNMT3A protein may be inhibited. In some embodiments, DNMT3A-mediated methylaytion is inhibited by deleting the DNMT3A gene or inhibiting expression of the DNMT3A gene.

In some embodiments of any of the modified immune effector cells described above, the level of functional DNMT3A protein in the cell may be decreased by about 50% or more. In some embodiments, the level of functional DNMT3A protein in the cell may be decreased by about 70% or more. In some embodiments, the level of functional DNMT3A protein in the cell may be decreased by about 90% or more. In some embodiments, the level of functional DNMT3A protein in the cell may be decreased by about 99% or more.

In certain aspects, the IL10 signaling pathway in the immune effector cell may be enhanced by subjecting the cell to an effective amount of an exogenous IL10 or a carrier comprising the exogenous IL10. In some embodiments, the exogenous IL10 may be a recombinant IL10. In some embodiments, the carrier may be a nanoparticle.

In some embodiments of any of the modified immune effector cells described above, the immune effector cell may have been activated and/or expanded ex vivo. In some embodiments, the immune effector cell may be subjected to the exogenous IL10 at the beginning of cell expansion. In some embodiments, the immune effector cell may be subjected to the exogenous IL10 more than once. In some embodiments, the IL10 signaling pathway may be enhanced by genetically modifying the immune effector cell to express IL10. In some embodiments, the IL10 may be expressed from a transgene encoding IL10 introduced into the immune effector cell. In some embodiments, the transgene encoding IL10 may be inserted into the DNMT3A locus. In some embodiments, the IL10 may be constitutively expressed.

In various embodiments, the modified immune effector cell of the disclosure may further comprise a transgene encoding a suicide gene.

In some embodiments, the transgene encoding IL10 or the suicide gene may be introduced into the immune effector cell using a viral vector, a non-viral vector or a physical means.

In some embodiments, the immune effector cell may be a T cell. As a non-limiting example, the T cell may be a CD8+ T cell, a CD4+ T cell, a cytotoxic T cell, an αβ T cell receptor (TCR) T cell, a natural killer T (NKT) cell, a γδ T cell, a memory T cell, a T-helper cell, or a regulatory T cell (Treg).

In some embodiments, the immune effector cell may be a Nature Killer (NK) cell.

In some embodiments, the modified immune effector cell, e.g., T cell, may further comprise at least one surface molecule capable of binding specifically to an antigen. As a non-limiting example, the antigen may be a tumor antigen, a viral antigen, a bacterial antigen, a fungal antigen, a parasite antigen, a prion antigen, or an antigen associated with an inflammation or an autoimmune disease. In some embodiments, the tumor antigen may be human epidermal growth factor receptor 2 (HER2), IL13Rα2, erythropoietin-producing human hepatocellular receptor A2 (EphA2), B7-H3 (CD276), disialoganglioside (GD2), CD19, CD22, CD123, or glucose regulatory protein 78 (GRP78).

In one aspect of any of the modified immune effector cells described above, the cell may further comprise a chimeric antigen receptor (CAR), an antigen specific T-cell receptor (TCR), a bispecific antibody, and/or a T cell antigen coupler (TAC). In some embodiments, the cell further may comprise a chimeric antigen receptor (CAR).

In certain aspects, the modified immune cell may further comprise a CAR comprising an extracellular antigen-binding domain, a transmembrane domain, and a cytoplasmic domain. In some embodiments, the extracellular antigen-binding domain may comprise an antibody or an antibody fragment. In some embodiments, the extracellular antigen-binding domain may comprise an scFv capable of binding to human epidermal growth factor receptor 2 (HER2). In various embodiments, the scFv capable of binding to HER2 may comprise the amino acid sequence SEQ ID NO: 17. In some embodiments, the extracellular antigen binding domain may comprise an scFv capable of binding to IL13Rα2. In various embodiments, the scFv capable of binding to IL13Rα2 may comprise the amino acid sequence SEQ ID NO: 29. In some embodiments, the extracellular antigen binding domain may comprise an scFv capable of binding to erythropoietin-producing human hepatocellular receptor A2 (EphA2). In various embodiments, the scFv capable of binding to EphA2 may comprise the amino acid sequence SEQ ID NO: 38.

In some embodiments, the extracellular antigen-binding domain may further comprise a leader sequence. In various embodiments, the leader sequence may comprise the amino acid sequence SEQ ID NO: 15.

In some embodiments, the transmembrane domain may be derived from CD3ζ, CD28, CD4, or CD8a. In various embodiments, the transmembrane domain may be derived from CD3ζ and optionally comprise the amino acid sequence SEQ ID NO: 23. In various embodiments, the transmembrane domain may be derived from CD28 and optionally comprise the amino acid sequence SEQ ID NO: 31. In various embodiments, the transmembrane domain may be derived from CD8a and optionally comprise the amino acid sequence SEQ ID NO: 49. In various embodiments, the transmembrane domain may be derived from CD4 and optionally comprise the amino acid sequence SEQ ID NO: 51.

In certain aspects of any of the modified effector cells disclosed above, the CAR may further comprise a linker domain between the extracellular antigen-binding domain and the transmembrane domain. In some embodiments, the linker domain may comprise a hinge region. As a non-limiting example, the hinge region may comprise the amino acid sequence SEQ ID NO: 19. As another non-limiting example, the hinge region may comprise the amino acid sequence SEQ ID NO: 40. In some embodiments, the linker domain may comprise the amino acid sequence SEQ ID NO: 21.

In various embodiments, the CAR cytoplasmic domain may comprise one or more lymphocyte activation domains. As a non-limiting example, the lymphocyte activation domain may be derived from DAP10, DAP12, Fc epsilon receptor I γ chain (FCER1G), CD3δ, CD3ε, CD3γ, CD3ζ, CD27, CD28, CD40, CD134, CD137, CD226, CD79A, ICOS, or MyD88. In certain embodiments, the lymphocyte activation domain may be derived from CD3ζ and optionally comprise the amino acid sequence SEQ ID NO: 25.

In various embodiments, the CAR cytoplasmic domain may comprise one or more co-stimulatory domains. In certain embodiments, the co-stimulatory domain may be derived from CD28 and optionally comprise the amino acid sequence SEQ ID NO: 33.

In one aspect of any of the modified immune effector cells disclosed herein, the immune effector cell may be an allogeneic cell. In another aspect of any of the modified immune effector cells disclosed herein, the immune effector cell may be an autologous cell. In some embodiments, the immune effector cell may be isolated from a subject having a disease. As a non-limiting example, the disease may be a cancer, an infectious disease, an inflammatory disorder, or an autoimmune disease. In some embodiments, the cancer may be a cancer expressing HER2, IL13Rα2, or EphA2. In certain embodiments, the cancer may be a HER2-positive breast cancer. In certain embodiments, the cancer may be an IL13Rα2-positive glioblastoma.

In some embodiments, any of the above-described immune effector cells may be derived from a blood, marrow, tissue, or a tumor sample.

In one aspect is provided a method of preserving multipotency of an immune effector cell, the method comprising deleting or modifying a DNA (cytosine-5)-methyltransferase 3A (DNMT3A) gene or gene product in the cell so that the DNMT3A-mediated de novo DNA methylation of the cell genome is inhibited; and, enhancing IL10 signaling pathway in the immune effector cell. In some embodiments, the DNMT3A gene in the immune effector cell may be deleted or modified as a result of an activity of a site-specific nuclease. In various embodiments, the site-specific nuclease is an RNA-guided endonuclease. Non-limiting examples of an RNA-guided endonuclease are Cas9 protein, Cpf1 (Cas12a) protein, C2cl protein, C2c3 protein, and C2c2 protein. In some embodiments, the RNA-guided endonuclease may be a Cas9 protein. In some embodiments, the Cas9 protein may be programmed with a guide RNA (gRNA) targeting DNMT3A. In certain embodiments, the Cas9 protein may be programmed with a gRNA that may comprise a nucleotide sequence encoded by CCTGCATGATGCGCGGCCCANGG (SEQ ID NO: 63). In certain embodiments, the Cas9 protein may be programmed with a gRNA that may comprise a nucleotide sequence encoded by CCTGCATGATGCGCGGCCCA (SEQ ID NO: 75). In certain embodiments, the Cas9 protein may be programmed with a gRNA that may comprise a nucleotide sequence encoded by GCATGATGCGCGGCCCAAGGNGG (SEQ ID NO: 68). In certain embodiments, the Cas9 protein may be programmed with a gRNA that may comprise a nucleotide sequence encoded by GCATGATGCGCGGCCCAAGG (SEQ ID NO: 76).

In some embodiments, the site-specific nuclease may be a zinc finger nuclease, a TALEN nuclease, or mega-TALEN nuclease.

In some embodiments of any of the methods of preserving multipotency of an immune effector cell described above, the DNMT3A gene product in the immune effector cell may be deleted or modified as a result of an activity of an RNA interference (RNAi) molecule or an antisense oligonucleotide. In some embodiments, the RNAi molecule may be a small interfering RNA (siRNA) or a small hairpin RNA (shRNA). In some embodiments, the site-specific nuclease or the RNAi molecule or the antisense oligonucleotide may be introduced into the immune effector cell via a viral vector, a non-viral vector or a physical means.

In some embodiments of any of the methods of preserving multipotency of an immune effector cell described above, the level of DNMT3A-mediated de novo DNA methylation of the cell genome may be inhibited by about 50% or more. In various embodiments, the level of DNMT3A-mediated de novo DNA methylation of the cell genome may be inhibited by about 70% or more. In various embodiments, the level of DNMT3A-mediated de novo DNA methylation of the cell genome may be inhibited by about 90% or more. In various embodiments, the level of DNMT3A-mediated de novo DNA methylation of the cell genome may be inhibited by about 99% or more.

In certain aspects of the above-described methods of preserving multipotency of an immune effector cell, the IL10 signaling pathway in the immune effector cell may be enhanced by subjecting the cell to an effective amount of an exogenous IL10 or a carrier comprising the exogenous IL10. In some embodiments, the exogenous IL10 may be a recombinant IL10. In some embodiments, the carrier may be a nanoparticle.

In certain aspects, any of the methods of preserving multipotency of an immune effector cell described above may further comprise activating and/or expanding of the immune effector cell. In some embodiments, the immune effector cell may be subjected to the exogenous IL10 at the beginning of cell expansion. In some embodiments, the immune effector cell may be subjected to the exogenous IL10 more than once. In some embodiments, the subjecting step may be conducted ex vivo. In some embodiments, the subjecting step may be conducted in vivo.

In some embodiments, the IL10 signaling pathway may be enhanced by genetically modifying the immune effector cell to express IL10. In some embodiments, IL10 may be expressed from a transgene encoding IL10 introduced into the immune effector cell. As a non-limiting example, the transgene encoding IL10 may be introduced into the immune effector cell using a viral vector, a non-viral vector or a physical means.

In certain aspects of any of the methods of preserving multipotency of an immune effector cell described above, the immune effector cell may be a T cell. Non-limiting examples of T cells are a CD8+ T cell, a CD4+ T cell, a cytotoxic T cell, an αβ T cell receptor (TCR) T cell, a natural killer T (NKT) cell, a γδ T cell, a memory T cell, a T-helper cell, and a regulatory T cell (Treg).

In some embodiments of any of the methods of preserving multipotency of an immune effector cell described above, the cell may further comprise at least one surface molecule capable of binding specifically to an antigen. In some embodiments, the cell further comprises a chimeric antigen receptor (CAR), an antigen specific T-cell receptor (TCR), a bispecific antibody and/or a T cell antigen coupler (TAC). In certain embodiments, the CAR, TCR, bispecific antibody and/or TAC may be expressed from a transgene encoding the CAR, TCR, bispecific antibody and/or TAC introduced into the immune effector cell. In some embodiments, the transgene encoding theCAR, TCR, bispecific antibody and/or TAC may be introduced into the immune effector cell using a viral vector, a non-viral vector or a physical means. As a non-limiting example, the viral vector may be a retroviral vector, an adenoviral vector, an adeno-associated viral (AAV) vector, a herpes viral vector, or a baculoviral vector. In some embodiments, the retroviral vector is a lentiviral vector.

In certain embodiments of any of the above-described methods, the non-viral vector may be a transposon. In some embodiments, the transposon may be a sleeping beauty transposon or PiggyBac transposon.

In some embodiments, the physical means may be electroporation, microinjection, magnetofection, ultrasound, a ballistic or hydrodynamic method, or a combination thereof.

In one aspect is provided a modified immune effector cell a pharmaceutical composition comprising any of the modified immune effector cell of the present disclosure and a pharmaceutically acceptable carrier and/or excipient.

In another one aspect is provided a method of treating a disease in a subject in need thereof comprising administering to the subject an effective amount of the modified immune effector cells or the pharmaceutical composition of the present disclosure. In some embodiments, the modified immune effector cell may be an autologous cell. In some embodiments, the modified immune effector cell may be an allogeneic cell. In some embodiments, the disease may be a cancer, an infectious disease, an inflammatory disorder, or an autoimmune disease. In some embodiments, the cancer may be a solid tumor. In some embodiments, the cancer may be breast, prostate, urinary bladder, skin, lung, ovary, sarcoma, or brain cancer. In some embodiments, the cancer may be a cancer expressing HER2, IL13Rα2, or EphA2. In various embodiments, the cancer may be a HER2-positive breast cancer. In various embodiments, the cancer may be an IL13Rα2-positive glioblastoma.

In certain embodiments of any of the methods of treating a subject in need thereof disclosed herein, the method may comprise isolating an immune effector cell from the subject or a donor; modifying a DNMT3A gene or gene product in the immune effector cell such that the DNMT3A-mediated de novo DNA methylation of the cell genome is inhibited enhancing the IL10 signaling pathway in the immune effector cell by either subjecting the immune effector cell to an exogenous IL10 or genetically modifying the immune effector cell to express IL10; and introducing the modified immune effector cell into the subject.

In some embodiments, the subjecting the immune effector cell to an exogenous IL10 may be carried out ex vivo. In some embodiments, the subjecting the immune effector cell to an exogenous IL10 may be carried out in vivo. In some embodiments, the exogenous IL10 may delivered to the immune effector cell in vivo in a carrier. As a non-limiting example, the wherein the carrier is an oncolytic virus, a lentivirus, or a nanoparticle.

In certain aspects of any of the methods of treating a subject disclosed herein, the method may further comprise genetically modifying the immune effector cell to express a chimeric antigen receptor (CAR) capable of binding specifically to an antigen.

In some embodiments of any of the methods of treating a subject disclosed herein, the subject may be human.

### BRIEF DESCRIPTION OF THE DRAWINGS

FIGS. 1A-1E show IL10 knockout (KO) in DNA methyltransferase 3 alpha (DNMT3A) edited chimeric antigen receptor (CAR) T-cells. FIG. 1A displays a table showing expression of genes upregulated by IL10 that overlap with DNMT3A targets. FIG. 1B shows targeted next generation sequencing (NGS) for DNMT3A and IL10 in different CAR T-cell populations. FIG. 1C shows a Western Blot confirming DNMT3A knock-out. FIG. 1D shows CAR transduction efficacy in gene edited CAR T-cells. FIG. 1E shows gene edited CAR T-cell viability at day 8-10 post transduction. FIG. 1F shows gene edited CAR T-cell expansion over 10 days. NT—non-transduced T-cell, Ctrl—T-cells transduced with double control guides to mimic double KO conditions; DNMT3A KO—Control and DNMT3A KO to mimic double KO condition; DKO—DNMT3A and IL-10 double knock-out.

FIGS. 2A-2H show IL10 promotes DNMT3A KO CAR T-cell survival. Activated human T-cells were electroporated with control (mCherry), DNMT3A, IL10, or DNMT3A and IL10 (DKO) ribonucleoproteins (RNPs). As shown in FIG. 2A, at day 3, gene edited T-cells were transduced with retroviral vector encoding HER2.ζCAR. CAR T-cells were incubated with U373 tumor cells at a 2:1 effector:target ratio in the presence of exogenous IL15 and restimulated with fresh tumor cells on a weekly basis until the T-cells stopped killing tumor cells, as shown in FIGS. 2B-2E. Cell culture supernatants were harvested 24 hours after each stimulation, and an IL10 ELISA was used to determine IL10 cytokine production (FIG. 2B). Gene edited CAR T-cell expansion after weekly stimulations with U373 cells for HER2.ζ-CAR is shown in FIG. 2C. Each graph represents one healthy donor. FIG. 2D (left panel) shows a summary graph of CAR T cell expansion (relative to Ctrl) for all donors up to 5 stimulations. n=4; ns=not significant; p<0.0001:****, 2-way ANOVA with Tukey's multiple comparisons test. FIG. 2D (middle panel) shows a summary graph of CAR T cell expansion (relative to Ctrl) for all donors in the presence of exogenous IL10. n=3; p<0.01:*, 2-way ANOVA with Tukey's multiple comparisons test. FIG. 2D (right panel) shows a summary graph of CAR T cell expansion (relative to Ctrl) for all donors, with exogenous IL10 added post 3^{rd} stimulation. n=3; p<0.0001:****, 2-way ANOVA with Tukey's multiple comparisons test. FIG. 2E shows normalized multipotency index (MPI) of Ctrl, IL-10 KO, DNMT3A KO and DKO CAR T cells were measured prior to stimulation and after four weeks of chronic stimulation. n=3; ns=not significant, p<0.001: ***, p<0.0001: ****, unpaired t-test. Each dot represents data from a single donor. FIG. 2F shows total number of differentially methylated regions (DMRs) between week 4 DNMT3A KO (D3A KO), IL10 KO, and DKO CAR T-cells. FIG. 2G shows representative methylation profiles of the CD28, LEF1, and TCF7 loci among week 4 Ctrl, DNMT3A KO, IL10 KO, and DKO CAR T-cells. FIG. 2H shows gene ontology analysis of DMRs between week 4 DNMT3A KO, IL10 KO, and DKO CAR T-cells.

FIGS. 3A-3B show genome wide comparison of control (Ctrl) and DNMT3A KO CAR T-cells with function and exhausted endogenous CD8 T-cell populations. FIG. 3A shows violin plot characterizing the methylation ratio of Control (Ctrl) KO, DNMT3A KO, IL10 KO, and DNMT3A+IL10 (DKO) CAR T-cells from three separate donors as comported to CD8 T-cell subsets from healthy human donors and tetramer positive (tet⁺) CD8 T-cells from HIV infected individuals. FIG. 3B shows principal component analysis (PCA) of whole genome methylation profiles showing segregation of week 4 post-stimulation DNMT3A-deficient CAR T-cells from CAR T-cells with intact DNMT3A activity.

FIGS. 4A-4C show DNMT3A mediated methylation programming occurs both early and late during chronic antigen stimulation. Differentially methylated regions (DMRs) between control (Ctrl) KO, DNMT3A KO, IL10 KO, and DNMT3A+IL10 double KO (DKO) CAR T-cells at week 0 and week 4 poststimulation. Individual CpG sites are represented by vertical lines with bottom bar indicating methylation and top bar indicating lack of methylation. DMRs are represented by a dashed box.

FIG. 5 shows IL10RA locus remains unmethylated near the promoter in the DNMT3A KO CAR T cells. Specifically, an enhancer upstream of the IL10RA promoter remains unmethylated in the KO CAR T cells relative to the control edited CD8+ CAR T cells. These data are consistent with the conclusion that DNMT3A deficient CAR T cells retain an ability to respond to IL 10 signaling. Representative methylation profile among week 4 control (Ctrl), DNMT3A KO, IL10 KO, and DNMT3A+IL10 double KO (DKO) CAR T-cells. Individual CpG sites are represented by vertical lines with bottom bar indicating methylation and top bar indicating lack of methylation. DMRs are represented by a dashed box.

### DETAILED DESCRIPTION

The present disclosure is based at least in part on the discovery that increased functionality and preserved multipotency of immune effector cells, e.g., CAR T cells wherein DNMT3A-mediated de novo DNA methylation of the cell genome is inhibited, can be coupled to enhanced IL10 signaling. IL10 is known as an immunosuppressive cytokine and in the CAR T cell field it is well accepted that presence of IL10 in the tumor microenvironment abolishes T-cell function (See e.g., Guo et al., Metabolic reprogramming of terminally exhausted CD8+ T cells by IL-10 enhances anti-tumor immunity. Nat Immunol. 2021 June;22(6):746-756; Ouyang et al., IL-10 Family Cytokines IL-10 and IL-22: from Basic Science to Clinical Translation. Immunity. 2019 Apr. 16; 50(4):871-89; and Wang et al., Targeting IL-10 Family Cytokines for the Treatment of Human Diseases. Cold Spring Harb Perspect Biol. 2019 Feb. 1; 11(2):a028548, each of which is incorporated herein by reference in its entirety for all purposes). Therefore, the discovery described herein is surprising and unexpected.

Enhancing the IL10 signaling pathway in such immune effector cells addresses the need in the art for immune effector cells with sustained proliferation and effector function, in particular, in the face of chronic antigen exposure, as well as preserved developmental potential; and, for optimized CAR T cell-based therapeutic strategies effective in the treatment of cancer. This approach can be used to preserve the developmental potential of immune effector cells used for adoptive cellular therapies during product manufacturing and/or supplement the effector cell response during application. Retention of a multipotent developmental status in immune effector cells utilized for immune checkpoint blockade and CAR modified cell therapies has been shown to be coupled to positive clinical outcomes. The data described in the Examples section below demonstrate in preclinical models that combining IL10 with approaches that block acquisition of tolerance epigenetic programs can enable less differentiated cells to persist in therapeutic settings.

### Definitions

The term “immune effector cell” as used herein refers to a cell that is involved in an immune response, e.g., in the promotion of an immune effector response. Non-limiting examples of immune effector cells include T cells (e.g., αβ T cells and γδ T cells), B cells, natural killer (NK) cells, natural killer T (NKT) cells, mast cells, and myeloid-derived phagocytes. Stem cells, such induced pluripotent stem cells (iPSCs), that are capable of differentiating into immune cells are also included here.

The terms “T cell” and “T lymphocyte” are interchangeable and used synonymously herein. As used herein, T cell includes thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a CD8+ T cell, a CD4+ T cell, a helper T cell or T-helper cell (HTL; CD4+ T cell), a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells and memory T cells. Also included are “αβ T cell receptor (TCR) T cells”, which refer to a population of T cells that possess a TCR composed of α- and β-TCR chains. Also included are “NKT cells”, which refer to a specialized population of T cells that express a semi-invariant αβ T-cell receptor, but also express a variety of molecular markers that are typically associated with NK cells, such as NK1.1. NKT cells include NK1.1+ and NK1.1-, as well as CD4+, CD4-, CD8+ and CD8—cells. The TCR on NKT cells is unique in that it recognizes glycolipid antigens presented by the MHC I-like molecule CD Id. NKT cells can have either protective or deleterious effects due to their abilities to produce cytokines that promote either inflammation or immune tolerance. Also included are “gamma-delta T cells (γδ T cells),” which refer to a specialized population that to a small subset of T cells possessing a distinct TCR on their surface, and unlike the majority of T cells in which the TCR is composed of two glycoprotein chains designated α- and β-TCR chains, the TCR in γδ T cells is made up of a γ-chain and a δ-chain. γδ T cells can play a role in immunosurveillance and immunoregulation, and were found to be an important source of IL-17 and to induce robust CD8+ cytotoxic T cell response. Also included are “regulatory T cells” or “Tregs”, which refer to T cells that suppress an abnormal or excessive immune response and play a role in immune tolerance. Tregs cells are typically transcription factor Foxp3-positive CD4+ T cells and can also include transcription factor Foxp3-negative regulatory T cells that are IL-10-producing CD4+ T cells.

The terms “natural killer cell” and “NK cell” are used interchangeable and used synonymously herein. As used herein, NK cell refers to a differentiated lymphocyte with a CD 16+CD56+ and/or CD57+ TCR-phenotype. NKs are characterized by their ability to bind to and kill cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate or inhibit the immune response.

The term “signaling molecule” as used herein, refers to any molecule that is capable of inducing a direct or indirect response in at least one cellular signaling pathway. The response may be stimulatory or inhibitory.

The term “switch receptor” used herein refers to a receptor that is capable of converting a potentially inhibitory signal into a positive signal. Switch receptors are also known as inverted cytokine receptors.

The term “chimeric antigen receptor” or “CAR” as used herein is defined as a cell-surface receptor comprising an extracellular target-binding domain, a transmembrane domain and a cytoplasmic domain, comprising a lymphocyte activation domain and optionally at least one co-stimulatory signaling domain, all in a combination that is not naturally found together on a single protein. This particularly includes receptors wherein the extracellular domain and the cytoplasmic domain are not naturally found together on a single receptor protein. The chimeric antigen receptors of the present invention are intended primarily for use with lymphocyte such as T cells and natural killer (NK) cells.

As used herein, the term “antigen” refers to any agent (e.g., protein, peptide, polysaccharide, glycoprotein, glycolipid, nucleic acid, portions thereof, or combinations thereof) molecule capable of being bound by a T-cell receptor. An antigen is also able to provoke an immune response. An example of an immune response may involve, without limitation, antibody production, or the activation of specific immunologically competent cells, or both. A skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components, organisms, subunits of proteins/antigens, killed or inactivated whole cells or lysates.

The term “antigen-binding moiety” refers to a target-specific binding element that may be any ligand that binds to the antigen of interest or a polypeptide or fragment thereof, wherein the ligand is either naturally derived or synthetic. Examples of antigen-binding moieties include, but are not limited to, antibodies; polypeptides derived from antibodies, such as, for example, single chain variable fragments (scFv), Fab, Fab′, F(ab′)2, and Fv fragments; polypeptides derived from T Cell receptors, such as, for example, TCR variable domains; secreted factors (e.g., cytokines, growth factors) that can be artificially fused to signaling domains (e.g., “zytokines”); and any ligand or receptor fragment (e.g., CD27, NKG2D) that binds to the antigen of interest. Combinatorial libraries could also be used to identify peptides binding with high affinity to the therapeutic target.

The terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910, each of which are incorporated by reference in their entirety for all purposes. Antibodies useful as a TCR-binding molecule include immunoglobulin molecules and immunologically active fragments of immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgM1, IgM2, IgA1 and IgA2) or subclass. Also included are “bispecific antibodies”, which refer to antibodies that are capable of binding to two different antigens or different epitopes of the same antigen.

The term “host cell” means any cell that contains a heterologous nucleic acid. The heterologous nucleic acid can be a vector (e.g., an expression vector). For example, a host cell can be a cell from any organism that is selected, modified, transformed, grown, used or manipulated in any way, for the production of a substance by the cell, for example the expression by the cell of a gene, a DNA or RNA sequence, a protein or an enzyme. An appropriate host may be determined. For example, the host cell may be selected based on the vector backbone and the desired result. By way of example, a plasmid or cosmid can be introduced into a prokaryote host cell for replication of several types of vectors. Bacterial cells such as, but not limited to DH5a, JM109, and KCB, SURE® Competent Cells, and SOLOPACK Gold Cells, can be used as host cells for vector replication and/or expression. Additionally, bacterial cells such as E. coli LE392 could be used as host cells for phage viruses. Eukaryotic cells that can be used as host cells include, but are not limited to yeast (e.g., YPH499, YPH500 and YPH501), insects and mammals. Examples of mammalian eukaryotic host cells for replication and/or expression of a vector include, but are not limited to, HeLa, NIH3T3, Jurkat, 293, COS, CHO, Saos, and PC12.

Host cells of the present invention include T cells and natural killer cells that contain the DNA or RNA sequences encoding the CAR and express the CAR on the cell surface. Host cells may be used for enhancing T cell activity, natural killer cell activity, treatment of cancer, and treatment of autoimmune disease.

The terms “activation” or “stimulation” means to induce a change in their biologic state by which the cells (e.g., T cells and NK cells) express activation markers, produce cytokines, proliferate and/or become cytotoxic to target cells. All these changes can be produced by primary stimulatory signals. Co-stimulatory signals can amplify the magnitude of the primary signals and suppress cell death following initial stimulation resulting in a more durable activation state and thus a higher cytotoxic capacity. A “co-stimulatory signal” refers to a signal, which in combination with a primary signal, such as TCR/CD3 ligation, leads to T cell and/or NK cell proliferation and/or upregulation or downregulation of key molecules.

The term “proliferation” refers to an increase in cell division, either symmetric or asymmetric division of cells. The term “expansion” refers to the outcome of cell division and cell death.

The term “differentiation” refers to a method of decreasing the potency or proliferation of a cell or moving the cell to a more developmentally restricted state.

The terms “express” and “expression” mean allowing or causing the information in a gene or DNA sequence to become produced, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as intracellular, extracellular, or transmembrane.

The term “transfection” means the introduction of a “foreign” (i.e., extrinsic, or extracellular) nucleic acid into a cell using recombinant DNA technology. The term “genetic modification” means the introduction of a “foreign” (i.e., extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences operably linked to polynucleotide encoding the chimeric antigen receptor, such as start, stop, promoter, signal, secretion, or other sequences used by a cell's genetic machinery. The gene or sequence may include nonfunctional sequences or sequences with no known function. A host cell that receives and expresses introduced DNA or RNA has been “genetically engineered.” The DNA or RNA introduced to a host cell can come from any source, including cells of the same genus or species as the host cell, or from a different genus or species.

The term “transduction” means the introduction of a foreign nucleic acid into a cell using a viral vector.

The terms “genetically modified” or “genetically engineered” refers to the addition of extra genetic material in the form of DNA or RNA into a cell.

The term “variant” as used herein refers to a modified polypeptide, protein, or polynucleotide that has substantial or significant sequence identity or similarity to a wild type or reference polypeptide, protein, or polynucleotide. The variant may retain the same, or have altered (e.g., improved, reduced, or abolished) biological activity relative to the wild type or reference polypeptide, protein, or polynucleotide of which it is a variant. The variant may contain an insertion, a deletion, a substitution of at least one amino acid residue or nucleotide.

As used herein, the term “variant” or “derivative” in the context of proteins or polypeptides (e.g., CAR constructs or domains thereof) may refer to: (a) a polypeptide that has at least 40%, 45%, 50%, 55%, 60%, 65%, 70%, 75%, 80%, 85%, 90%, 95%, 98% or 99% sequence identity to the polypeptide it is a variant or derivative of; (b) a polypeptide encoded by a nucleotide sequence that has at least 40%, 45%, 50%, 55%, 60%, 65%, 70%, 75%, 80%, 85%, 90%, 95%, 98% or 99% sequence identity to a nucleotide sequence encoding the polypeptide it is a variant or derivative of; (c) a polypeptide that contains 1, 2, 3, 4, 5, 6, 7, 8, 9, 10, 11, 12, 13, 14, 15, 16, 17, 18, 19, 20 or more amino acid mutations (i.e., additions, deletions and/or substitutions) relative to the polypeptide it is a variant or derivative of, (d) a polypeptide encoded by nucleic acids can hybridize under high, moderate or typical stringency hybridization conditions to nucleic acids encoding the polypeptide it is a variant or derivative of, (e) a polypeptide encoded by a nucleotide sequence that can hybridize under high, moderate or typical stringency hybridization conditions to a nucleotide sequence encoding a fragment of the polypeptide, it is a variant or derivative of, of at least 20 contiguous amino acids, at least 30 contiguous amino acids, at least 40 contiguous amino acids, at least 50 contiguous amino acids, at least 75 contiguous amino acids, at least 100 contiguous amino acids, at least 125 contiguous amino acids, or at least 150 contiguous amino acids; or (f) a fragment of the polypeptide it is a variant or derivative of.

Percent sequence identity can be determined using any method known to one of skill in the art. In a specific embodiment, the percent identity is determined using the “Best Fit” or “Gap” program of the Sequence Analysis Software Package (Version 10; Genetics Computer Group, Inc., University of Wisconsin Biotechnology Center, Madison, Wisconsin). Information regarding hybridization conditions (e.g., high, moderate, and typical stringency conditions) have been described, see, e.g., U.S. Patent Application Publication No. US 2005/0048549 (e.g., paragraphs 72-73).

Percent sequence identity can be determined using a global alignment between two sequences. As used herein, the term “global alignment” refers to an alignment of residues between two amino acid or nucleic acid sequences along their entire length, introducing gaps as necessary if the two sequences do not have the same length, to achieve a maximum percent identity. A global alignment can be created using the global alignment tool “Needle” from the online European Molecular Biology Open Software Suite (EMBOSS) (see ebi.ac.uk/Tools/psa/emboss_needle/) or the global alignment tool “BLAST® Global Alignment” from the National Center for Biotechnology Information (NCBI) (see blast.ncbi.nlm.nih.gov/Blast.cgi?PROGRAM=blastn&PAGE_TYPE=BlastSearch&PROG_DEF AULTS=on&BLAST_INIT=GlobalAln&BLAST_SPEC=GlobalAln&BLAST_PROGRAMS=bl astn). Both of these global alignment tools incorporate the Needleman-Wunsch algorithm (Needleman, S. B. & Wunsch, C.D. (1970) “A general method applicable to the search for similarities in the amino acid sequences of two proteins.” J. Mol. Biol. 48:443-453). In a preferred embodiment, a global alignment of nucleotide sequences using BLAST Global Alignment uses the following default parameters: match score=2; mismatch score=−3; Gap Cost Existence score=5; Gap Cost Extension Score=2. In a preferred embodiment, a global alignment of protein sequences using BLAST Global Alignment uses the following default parameters: Gap Cost Existence=11; Gap Cost Extension=1.

The terms “vector”, “cloning vector” and “expression vector” mean the vehicle by which a DNA or RNA sequence (e.g., a foreign gene) can be introduced into a host cell, so as to genetically modify the host and promote expression (e.g., transcription and translation) of the introduced sequence. Vectors include plasmids, synthesized RNA and DNA molecules, phages, viruses, etc. In some embodiments, the vector is a viral vector such as, but not limited to, an adenoviral, adeno-associated viral (AAV) vector, alphaviral, herpes, lentiviral, retroviral, baculoviral, or vaccinia vector. In some embodiments, the vector is a non-viral vector. In some embodiments, the non-viral vector is a transposon such as, but not limited to, a sleeping beauty transposon or a PiggyBac transposon.

The term “regulatory element” refers to any cis-acting genetic element that controls some aspect of the expression of nucleic acid sequences. In some embodiments, the term “promoter” comprises essentially the minimal sequences required to initiate transcription. In some embodiments, the term “promoter” includes the sequences to start transcription, and in addition, also include sequences that can upregulate or downregulate transcription, commonly termed “enhancer elements” and “repressor elements”, respectively.

As used herein, the term “operatively linked,” and similar phrases, when used in reference to nucleic acids or amino acids, refer to the operational linkage of nucleic acid sequences or amino acid sequence, respectively, placed in functional relationships with each other. For example, an operatively linked promoter, enhancer elements, open reading frame, 5′ and 3′ UTR, and terminator sequences result in the accurate production of a nucleic acid molecule (e.g., RNA). In some embodiments, operatively linked nucleic acid elements result in the transcription of an open reading frame and ultimately the production of a polypeptide (i.e., expression of the open reading frame). As another example, an operatively linked peptide is one in which the functional domains are placed with appropriate distance from each other to impart the intended function of each domain.

By “enhance” or “promote,” or “increase” or “expand” or “improve” refers generally to the ability of a composition contemplated herein to produce, elicit, or cause a greater physiological response (i.e., downstream effects) compared to the response caused by either vehicle or a control molecule/composition. A measurable physiological response may include an increase in T cell expansion, activation, effector function, persistence, and/or an increase in antitumor activity (e.g., cancer cell death and cancer cell killing ability), among others apparent from the understanding in the art and the description herein. In some embodiments, an “increased” or “enhanced” amount can be a “statistically significant” amount, and may include an increase that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. In some embodiments, a “decrease” or “reduced” amount can be a “statistically significant” amount, and may include a decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

The terms “inhibit” or “inhibition” as used herein refer to reducing a function or activity to an extent sufficient to achieve a desired biological or physiological effect. Inhibition may be complete or partial.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The term “protein” is used herein encompasses all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

The terms “nucleic acid”, “nucleotide”, and “polynucleotide” encompass both DNA and RNA unless specified otherwise. By a “nucleic acid sequence” or “nucleotide sequence” is meant the nucleic acid sequence encoding an amino acid, the term may also refer to the nucleic acid sequence including the portion coding for any amino acids added as an artifact of cloning, including any amino acids coded for by linkers

The terms “patient”, “individual”, “subject”, and “animal” are used interchangeably herein and refer to mammals, including, without limitation, human and veterinary animals (e.g., cats, dogs, cows, horses, sheep, pigs, etc.) and experimental animal models. In a preferred embodiment, the subject is a human.

The term “carrier” refers to a diluent, adjuvant, excipient, or vehicle with which the compound is administered. Such pharmaceutical carriers can be sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E.W. Martin.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

If aspects of the disclosure are described as “comprising” a feature, or versions there of (e.g., comprise), embodiments also are contemplated “consisting of” or “consisting essentially of” the feature.

The practice of the present invention employs, unless otherwise indicated, conventional techniques of statistical analysis, molecular biology (including recombinant techniques), microbiology, cell biology, and biochemistry, which are within the skill of the art. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ. Additional techniques are explained, e.g., in U.S. Pat. No. 7,912,698 and U.S. Patent Appl. Pub. Nos. 2011/0202322 and 2011/0307437.

The technology illustratively described herein suitably may be practiced in the absence of any element(s) not specifically disclosed herein.

The terms and expressions which have been employed are used as terms of description and not of limitation, and use of such terms and expressions do not exclude any equivalents of the features shown and described or portions thereof, and various modifications are possible within the scope of the technology claimed.

Certain methods and compositions described in PCT Applications WO2020/210365 and WO2020/222987 may be useful in various aspects of the present disclosure. Both of WO2020/210365 and WO2020/222987 are incorporated herein by reference in their entirety.

### Modified Immune Effector Cells

In one aspect, the invention provides a modified immune effector cell with enhanced immune cell function. In particular, the immune effector cell is modified such that the DNA (cytosine-5)-methyltransferase 3A (DNMT3A)-mediated de novo DNA methylation of the cell genome is inhibited and IL10 signaling pathway is enhanced.

In one aspect, the invention provides a modified immune effector cell with enhanced immune cell function. In particular, the immune effector cell is modified such that the DNA (cytosine-5)-methyltransferase 3A (DNMT3A)-mediated de novo DNA methylation of the cell genome is inhibited, IL10 signaling pathway is enhanced, and optionally STAT5 signaling pathway is activated.

In some embodiments, the immune effector cell is a T cell. T cells may include, but are not limited to, thymocytes, naive T lymphocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), a tumor infiltrating cytotoxic T cell (TIL; CD8+ T cell), CD4+CD8+ T cell, or any other subset of T cells. Other illustrative populations of T cells suitable for use in particular embodiments include naive T cells memory T cells, and NKT cells.

In some embodiments, the T cell may be a CD8+ T cell, a CD4+ T cell, a cytotoxic T cell, an αβ T cell receptor (TCR) T cell, a natural killer T (NKT) cell, a γδ T cell, a memory T cell, a T-helper cell, or a regulatory T cell (Treg).

The modification may be applied to all forms of T cell therapies, which include but not limited to therapies with i) T cells that express a chimeric antigen receptor (CAR); ii) T cells that express an endogenous αβ TCR or an endogenous γδ TCR, which may be specific for, e.g., a peptide derived from viral or tumor-associated antigens (including neoantigens); iii) T cells that transgenically express an αβ TCR or an endogenous γδ TCR, which may be specific for, e.g., a peptide derived from viral or tumor-associated antigens (including neoantigens); iv) T cells that transgenically express bispecific antibodies, which recognize viral or tumor-associated antigens (including neoantigens)/or a peptide derived from them and an activating molecule expressed on T cells such as CD3; and/or v) T cells that are generated via stimulation with for examples but not limited to peptides, antigen presenting and/or artificial antigen presenting cells (in vitro sensitized [IVS] T cell therapy). Lastly, T cell therapies in which the therapeutic genes are delivered in vivo are also included (in vivo T cell therapy).

In some embodiments, the immune effector cell is a natural killer (NK) cell. NK cell refers to a differentiated lymphocyte with a CD3−CD16+, CD3−CD56+, CD16+CD56+ and/or CD57+ TCR-phenotype.

In some embodiments, the immune effector cell is a stem cell that is capable of differentiating into an immune cell. The stem cell may be an induced pluripotent stem cell (iPSC).

DNA (cytosine-5)-methyltransferase 3A (DNMT3A) is an enzyme that catalyzes the addition of methyl groups to cytosine residues of CpG structures in DNA. The enzyme is encoded in humans by the DNMT3A gene. This enzyme is responsible for de novo DNA methylation. Such function may be different from maintenance DNA methylation which ensures the fidelity of replication of inherited epigenetic patterns. The DNMT3A-mediated de novo DNA methylation is critical in DNA imprinting and modulation of gene expression.

In some embodiments, the enzymatic activity of the DNMT3A protein is inhibited in the cell. The enzymatic activity of the DNMT3A protein may be inhibited by exposing the cell to a DNMT3A active site inhibitor. Although not wishing to be bound by theory, the methyl-transfer reaction carried out by a DNA methyltransferase is typically initiated by nucleophilic attack from a catalytic cysteine in the active site. The catalytic cysteine is highly conserved among cytosine methyltransferases. When the catalytic cysteine is mutated or blocked the enzymatic activity of the DNMT3A protein can be inhibited, although binding may still occur. The catalytic cysteine of human DNMT3A has been identified to be C710 (Zhang, Z. M. et al., Nature. 2018; 554(7692): 387-391, which is incorporated herein by reference in its entirety for all purposes). Examples of DNMT3A active site inhibitors that may be used in the present invention include 5-azacytidine, Decitabine, Zebularine, 5-fluoro-2′-deoxycytidine, as well as other cytidine analogs known in the art. A further example of a DNMT3A active site inhibitor includes RG108.

In some embodiments, the DNMT3A gene is mutated in the DNMT3A catalytic domain so that the enzymatic activity of the DNMT3A protein is inhibited. As a non-limiting example, a catalytic cysteine in the catalytic domain may be mutated in a way that the enzymatic reaction can no longer occur.

In some embodiments, the expression of the DNMT3A gene is inhibited.

In some embodiments, the level of functional DNMT3A protein in the cell is decreased by about 50% or more. The level of functional DNMT3A protein in the cell may be decreased by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level of functional DNMT3A protein in the cell may be decreased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the level of functional DNMT3A protein in the cell is decreased by about 70% or more. In some embodiments, the level of functional DNMT3A protein in the cell is decreased by about 90% or more. In some embodiments, the level of functional DNMT3A protein in the cell is decreased by about 99% or more.

In some embodiments, the DNMT3A gene is deleted or defective so that no detectable wild-type DNMT3A protein is produced. The DNMT3A gene may be deleted or become defective using the methods described herein.

In some embodiments, the IL10 signaling pathway in any of the various immune effector cells disclosed herein is modified. In some embodiments, the IL10 signaling pathway is enhanced in the cell. In some embodiments, the IL10 signaling pathway is enhanced by subjecting the cell to an effective amount of an exogenous IL10. In some embodiments, the exogenous IL10 may be a recombinant IL10. In some embodiments, the IL10 signaling pathway is enhanced by subjecting the cell to an effective amount of a carrier comprising the exogenous IL10. A non-limiting example of a carrier is a nanoparticle.

In some embodiments, the STATS signaling pathway is activated by a signaling molecule. The signaling molecule may be a common gamma chain cytokine. Non-limiting examples of cytokines that may be used in the methods described herein include IL-15, IL-7, IL-2, IL-4, IL-9, and IL-21. The cytokine may be a native or modified cytokine. In some embodiments, the signaling molecule is IL-15. In some embodiments, the signaling molecule is IL-7.

In some embodiments, IL10 signaling pathway and/or STAT5 signaling pathway is enhanced or activated in any of the immune effector cell disclosed herein.

In some embodiments, the immune effector cell has been activated and/or expanded ex vivo.

In some embodiments, the immune effector cell is subjected to the exogenous IL10 at the beginning of the expansion. In some embodiments, the immune effector cell may be subjected to the exogenous IL10 more than once. In some embodiments, the immune effector cell may be subject to exogenous IL10 2 times, 3 times, 4 times, 5 times, 6 times, 7 times, 8 times, or more than 8 times. The immune effector cell may be subject to exogenous IL10 at a frequency of every 8 hours, every 12 hours, every 16 hours, every 24 hours, every 2 days, every 3 days, every 4 days, every 5 days, every 6 days, every 7 days, every 8 days, every 8 days, every 10 days, once a week, twice a week, biweekly, once a month, twice a month, 3 times a month, 4 times a month, or 5 times a month.

In some embodiments, the IL10 signaling pathway may be enhanced by genetically modifying the immune effector cell to express IL10 using any of the methods described herein. In some embodiments, the IL-10 may be expressed from a transgene encoding IL1-0 introduced into the immune effector cell. In some embodiments, the transgene encoding the IL10 is inserted into the DNMT3A locus.

In some embodiments, IL10 may be constitutively expressed.

In some embodiments, the immune effector cell of the present disclosure may further comprise a transgene encoding a suicide gene. The invention thus represents an efficient, reliable, and targeted approach for eliminating immune effector cells that may have been, e.g., administered to a subject without damaging surrounding cells and tissues, suitable for various cellular therapeutics.

In some embodiments, the immune effector cell of the present disclosure may further comprise a transgene encoding a switch receptor. A switch receptor (also known as inverted cytokine receptor), which is capable of converting a potentially inhibitory signal into a positive signal, is also contemplated by the present invention. Non-limiting examples of switch receptors that may also be used in the methods described herein include an IL4/IL7 receptor and an IL4/IL2 receptor. Such receptors may be generated as described in Bajgain, P. et al., J Immunother Cancer. 2018;6(1):34 and Wilkie, S. et al., J Biol Chem. 2010; 285(33):25538-44, both of which are incorporated herein by reference in their entirety for all purposes.

In some embodiments, the transgene encoding the suicide gene and/or switch receptor is introduced into the immune effector cell using a vector. Vectors include plasmids, synthesized RNA and DNA molecules, phages, viruses, etc. In certain embodiments, the vector is a viral vector such as, but not limited to, an adenoviral, adeno-associated, alpha viral, herpes, lentiviral, retroviral, or vaccinia vector.

In some embodiments, the transgene encoding the suicide gene and/or switch receptor is introduced into the immune effector cell using a viral vector such as, but not limited to, an adenoviral vector, an adeno-associated viral (AAV) vector, a herpes viral vector, or a baculoviral vector. In some embodiments, the retroviral vector is a lentiviral vector. In some embodiments, the transgene encoding the suicide gene and/or switch receptor is introduced into the immune effector cell using a non-viral vector. In some embodiments, the non-viral vector is a transposon such as, but not limited to a sleeping beauty transposon or a PiggyBac transposon. In some embodiments, the transgene encoding the suicide gene and/or switch receptor is introduced into the immune effector cell using or a physical means. In some embodiments, the physical means electroporation, microinjection, magnetofection, ultrasound, a ballistic or hydrodynamic method, or a combination thereof.

In some embodiments, the transgene encoding the IL10 is introduced into the immune effector cell using a viral vector such as, but not limited to, an adenoviral vector, an adeno-associated viral (AAV) vector, a herpes viral vector, or a baculoviral vector. In some embodiments, the retroviral vector is a lentiviral vector. In some embodiments, the transgene encoding the IL10 is introduced into the immune effector cell using a non-viral vector. In some embodiments, the non-viral vector is a transposon such as, but not limited to a sleeping beauty transposon or a PiggyBac transposon In some embodiments, the transgene encoding IL10 is introduced into the immune effector cell using or a physical means. In some embodiments, the physical means is electroporation, microinjection, magnetofection, ultrasound, a ballistic or hydrodynamic method, or a combination thereof. In some embodiments, the STAT5 signaling pathway is activated by modifying the immune effector cell to express a constitutively active cytokine receptor or a switch receptor. Constitutively active cytokine receptors may trigger the activation of a cytokine signaling cascade even in the absence of extracellular cytokine. This may circumvent the need for providing extracellular cytokines to the immune effector cell. A non-limiting example of a constitutively active cytokine receptor is a constitutively active IL7 receptor (C7R). Such constitutively active cytokine receptor may be generated using methods described in Shum T et al. Cancer Discov. 2017; 7(11):1238-1247, which is incorporated herein in its entirety for all purposes.

In some embodiments, the modified immune effector cell further comprises at least one surface molecule capable of binding specifically to an antigen. The antigen may be a tumor antigen, a viral antigen, a bacterial antigen, a fungal antigen, a parasite antigen, a prion antigen, or an antigen associated with an inflammation or an autoimmune disease.

In some embodiments, the antigen is a tumor antigen. Non-limiting examples of tumor antigens that may be targeted by the modified immune effector cell described herein include human epidermal growth factor receptor 2 (HER2), interleukin-13 receptor subunit alpha-2 (IL-13Rα2), ephrin type-A receptor 2 (EphA2), A kinase anchor protein 4 (AKAP-4), adrenoceptor beta 3 (ADRB3), anaplastic lymphoma kinase (ALK), immunoglobulin lambda-like polypeptide 1 (IGLL1), androgen receptor, angiopoietin-binding cell surface receptor 2 (Tie 2), B7-H3 (CD276), bone marrow stromal cell antigen 2 (BST2), carbonic anhydrase IX (CAIX), CCCTC-binding factor (Zinc Finger Protein)-like (BORIS), CD171, CD179a, CD24, CD300 molecule-like family member f (CD300LF), CD38, CD44v6, CD72, CD79a, CD79b, CD97, chromosome X open reading frame 61 (CXORF61), claudin 6 (CLDN6), CS-1 (CD2 subset 1, CRACC, SLAMF7, CD319, or 19A24), C-type lectin domain family 12 member A (CLECi2A), C-type lectin-like molecule-1 (CLL-1), Cyclin B 1, Cytochrome P450 1B 1 (CYP1B 1), EGF-like module-containing mucin-like hormone receptor-like 2 (EMR2), epidermal growth factor receptor (EGFR), ERG (transmembrane protease, serine 2 (TMPRSS2) ETS fusion gene), ETS translocation-variant gene 6, located on chromosome 12p (ETV6-AML), Fc fragment of IgA receptor (FCAR), Fc receptor-like 5 (FCRL5), Fms-like tyrosine kinase 3 (FLT3), Folate receptor beta, Fos-related antigen 1, Fucosyl GM1, G protein-coupled receptor 20 (GPR20), G protein-coupled receptor class C group 5, member D (GPRC5D), ganglioside GD3, ganglioside GM3, glycoceramide (GloboH), Glypican-3 (GPC3), Hepatitis A virus cellular receptor 1 (HAVCR1), hexasaccharide portion of globoH, high molecular weight-melanoma-associated antigen (HMWMAA), human Telomerase reverse transcriptase (hTERT), interleukin 11 receptor alpha (IL-I IRa), KIT (CD 117), leukocyte-associated immunoglobulin-like receptor 1 (LAIR1), leukocyte immunoglobulin-like receptor subfamily A member 2 (LILRA2), Lewis(Y) antigen, lymphocyte antigen 6 complex, locus K 9 (LY6K), lymphocyte antigen 75 (LY75), lymphocyte-specific protein tyrosine kinase (LCK), mammary gland differentiation antigen (NY-BR-1), melanoma cancer testis antigen-1 (MAD-CT-1), melanoma cancer testis antigen-2 (MAD-CT-2), melanoma inhibitor of apoptosis (ML-IAP), mucin 1, cell surface associated (MUC1), N-acetyl glucosaminyl-transferase V (NA17), neural cell adhesion molecule (NCAM), o-acetyl-GD2 ganglioside (OAcGD2), olfactory receptor 51E2 (OR51E2), p53 mutant, paired box protein Pax-3 (PAX3), paired box protein Pax-5 (PAX5), pannexin 3 (PANX3), placenta-specific 1 (PLAC1), platelet-derived growth factor receptor beta (PDGFR-beta), Polysialic acid, proacrosin binding protein sp32 (OY-TES 1), prostate stem cell antigen (PSCA), Protease Serine 21 (PRSS21), Proteasome (Prosome, Macropain) Subunit, Beta Type, 9 (LMP2), Ras Homolog Family Member C (RhoC), sarcoma translocation breakpoints, sialyl Lewis adhesion molecule (sLe), sperm protein 17 (SPA17), squamous cell carcinoma antigen recognized by T cells 3 (SART3), stage-specific embryonic antigen-4 (SSEA-4), synovial sarcoma, X breakpoint 2 (SSX2), TCR gamma alternate reading frame protein (TARP), TGS5, thyroid stimulating hormone receptor (TSHR), Tn antigen (Tn Ag), tumor endothelial marker 1 (TEM1/CD248), tumor endothelial marker 7-related (TEM7R), uroplakin 2 (UPK2), vascular endothelial growth factor receptor 2 (VEGFR2), v-myc avian myelocytomatosis viral oncogene neuroblastoma derived homolog (MYCN), Wilms tumor protein (WT1), and X Antigen Family, Member 1A (XAGE1), or a fragment or variant thereof. Additional antigens that may be targeted by the extracellular target-binding domain include, but are not limited to, carbonic anhydrase EX, alpha-fetoprotein, A3, antigen specific for A33 antibody, Ba 733, BrE3-antigen, CA125, CD1, CD1a, CD3, CD5, CD15, CD16, CD19, CD20, CD21, CD22, CD23, CD25, CD30, CD33, CD38, CD45, CD74, CD79a, CD80, CD123, CD138, B7-H3 (CD276), disialoganglioside (GD2), GRP78, colon-specific antigen-p (CSAp), CEA (CEACAM5), CEACAM6, CSAp, EGFR, EGP-I, EGP-2, Ep-CAM, EphAl, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le- Y, macrophage inhibition factor (MIF), MAGE, MUC1, MUC2, MUC3, MUC4, NCA66, NCA95, NCA90, antigen specific for PAM-4 antibody, placental growth factor, p53, prostatic acid phosphatase, PSA, PSMA, RS5, S100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In some embodiments, the tumor antigen targeted by the modified immune effector cell is HER2, IL13Rα2, or EphA2, or a fragment or variant thereof.

In some embodiments, the tumor antigen targeted by the modified immune effector cell is HER2. Human epidermal growth factor receptor 2 (HER2), also referred to as HER2/neu, receptor tyrosine-protein kinase erbB-2, CD340 (cluster of differentiation 340), proto-oncogene Neu, or ERBB2, is a membrane tyrosine kinase and oncogene that is overexpressed in some types of cancer.

In some embodiments, the tumor antigen targeted by the modified immune effector cell is IL13Rα2. Interleukin-13 receptor subunit alpha-2 (IL13Rα2), also referred to as CD213A2 (cluster of differentiation 213A2), is a membrane bound protein that in humans is encoded by the IL13RA2 gene.

In some embodiments, the tumor antigen targeted by the modified immune effector cell is EphA2. Ephrin type-A receptor 2 (EphA2), also referred to as Eck (epithelial cell kinase), Myk2, or Sek2, is a member of the Eph receptor tyrosine kinase family which binds Ephrins A1, 2, 3, 4, and 5.

In some embodiments, the tumor antigen targeted by the modified immune effector cell is B7-H3 (CD276), disialoganglioside (GD2), CD19, CD22, CD123, CD33, or GRP78. In some embodiments, the tumor antigen targeted by the modified immune effector cell is B7-H3 (CD276). In some embodiments, the tumor antigen targeted by the modified immune effector cell is disialoganglioside (GD2). In some embodiments, the tumor antigen targeted by the modified immune effector cell is CD19. In some embodiments, the tumor antigen targeted by the modified immune effector cell is CD22. In some embodiments, the tumor antigen targeted by the modified immune effector cell is CD123. In some embodiments, the tumor antigen targeted by the modified immune effector cell is GRP78.

In some embodiments, the modified immune effector cell further comprises a chimeric antigen receptor (CAR), an antigen specific T-cell receptor, a bispecific antibody, and/or a T cell antigen coupler (TAC).

In some embodiments, the modified immune effector cell further comprises an antigen specific T-cell receptor. Antigen specific T-cell receptors are T-cell receptors (TCRs) that are specific for recognizing a particular antigen. In some embodiments, the modified immune effector cell comprises a T cell receptor (TCR), or a functional fragment thereof. By way of a non-limiting example, a functional fragment of a TCR may specifically bind to a particular antigen (or epitope) while retaining the capability to specifically bind to the antigen (or epitope). In various embodiments, a functional fragment of a TCR may comprise at least one complementary determining regions (CDRs) of the alpha chain and/or beta chain of the TCR. In various embodiments, a functional fragment of a TCR may comprise two or more complementary determining regions (CDRs) of the alpha chain and/or beta chain of the TCR.

In some embodiments, the TCR disclosed herein may comprise, for example, one or more of an alpha (α) chain of a TCR, a beta (β) chain of a TCR, a delta (δ) chain of a TCR, a gamma (γ) chain of a TCR, or a combination thereof. In some embodiments, the TCR may further comprise a constant region. The constant region may be derived from any suitable species such as, e.g., human or mouse.

In some embodiments, the TCR may comprise an alpha chain and/or a beta chain of the TCR. In some embodiments, the TCR may comprises, e.g., constant regions of alpha and/or beta chains of the TCR.

In some embodiments, the antigen specific TCR may recognize, without limitation, any of the antigens (e.g., an antigen(s) on a cancer cell) disclosed herein. In various embodiments, the TCR of the disclosure may specifically bind to an antigen selected from, for example, CD7, CD74, CDS, CEA, EGP-2, EGP-40, EpCAM, erb-B2,3,4, FBP, Fetal acetylcholine receptor, folate receptor-a, GD2, GD3, HER2, hTERT, IL-13R-a2, KDR, K-light chain, LeY, L1 cell, MAGE-A1, Mesothelin, MUC1, MUC16, NKG2D ligands, NY-ESO-1, oncofetal antigen (h5T4), PSCA, PSMA, ROR1, TAG-72, VEGF-R2, and WT-1.

In some embodiments, the modified immune effector cell further comprises a bispecific antibody. Bispecific antibodies are antibodies that are capable of binding to two different antigens or different epitopes of the same antigen. For example, the modified immune effector cell may comprise a bispecific antibody that is capable of binding to an molecule on the immune effector cell and is also capable of binding to an antigen on a target cell.

### Chimeric Antigen Receptor

In some embodiments, the modified immune effector cell further comprises a chimeric antigen receptor (CAR).

CARs are typically comprised primarily of 1) an extracellular antigen-binding domain comprising an extracellular antigen-binding moiety or domain, such as a single-chain variable fragment (scFv) derived from an antigen-specific monoclonal antibody, and 2) a lymphocyte activation domain, such as the ζ-chain from the T cell receptor CD3. These two regions are fused together via a transmembrane domain. Upon transduction, the lymphocyte expresses the CAR on its surface, and upon contact and ligation with the target antigen, it signals through the lymphocyte activation domain (e.g., CD3ζ chain) inducing cytotoxicity and cellular activation.

Constructs with only the antigen-specific binding region together with the lymphocyte activation domain are termed first-generation CARs. While activation of lymphocytes through a lymphocyte activation domain such as CD3ζ is sufficient to induce tumor-specific killing, such CARs fail to optimally induce T cell proliferation and survival in vivo. The second-generation CARs added co-stimulatory polypeptides to boost the CAR-induced immune response. For example, the co-stimulating polypeptide CD28 signaling domain was added to the CAR construct. This region generally contains the transmembrane region of the co-stimulatory peptide (in place of the CD3ζ transmembrane domain) with motifs for binding other molecules such as PI3K and Lck. T cells expressing CARs with only CD3ζ vs CARs with both CD3ζ and a co-stimulatory domain (e.g., CD28) demonstrated the CARs expressing both domains achieve greater activity. The most commonly used co-stimulating molecules include CD28 and 4-1BB, which promotes both T cell proliferation and cell survival. The third-generation CAR includes three signaling domains (e.g., CD3ζ, CD28, and 4-1BB), which further improves lymphocyte cell survival and efficacy. Examples of third-generation CARs include CD19 CARs, most notably for the treatment of chronic lymphocytic leukemia (Milone, M. C., et al., (2009) Mol. Ther. 17:1453-1464; Kalos, M., et al., Sci. Transl. Med. (2011) 3:95ra73; Porter, D., et al., (2011) N. Engl. J. Med. 365: 725-533, each of which is herein incorporated by reference in their entirety for all purposes). Studies in three patients showed impressive function, expanding more than a 1000-fold in vivo, and resulted in sustained remission in all three patients.

In some embodiments, the CAR expressed by a modified immune effector cell described herein comprises an extracellular antigen-binding domain and a transmembrane domain. In some embodiments, the CAR further comprises a cytoplasmic domain. Each domain is fused in frame.

In some embodiments, the CAR expressed by a modified immune effector cell described herein is a first-generation CAR. In some embodiments, the CAR expressed by a modified immune effector cell described herein is a second-generation CAR.

### Extracellular Antigen-Binding Domain of the CAR

The choice of antigen-binding domain depends upon the type and number of antigens that define the surface of a target cell. For example, the antigen-binding domain may be chosen to recognize an antigen that acts as a cell surface marker on target cells associated with a particular disease state. In some embodiments, the CARs can be genetically modified to target a tumor antigen of interest by way of engineering a desired antigen-binding domain that specifically binds to an antigen (e.g., on a cancer cell). Non-limiting examples of cell surface markers that may act as targets for the antigen-binding domain in the CAR include those associated with viral, bacterial, and parasitic infections, autoimmune disease, and cancer cells.

In some embodiments, the extracellular antigen-binding domain comprises an antigen-binding polypeptide or functional variant thereof that binds to an antigen. In some embodiments, the antigen-binding polypeptide is an antibody or an antibody fragment that binds to an antigen.

In some embodiments, the antigen-binding polypeptide can be monomeric or multimeric (e.g., homodimeric or heterodimeric), or associated with multiple proteins in a non-covalent complex. In some embodiments, the extracellular target-binding domain may consist of an Ig heavy chain. In some embodiments, the Ig heavy chain can be covalently associated with Ig light chain (e.g., via the hinge and optionally the CH1 region). In some embodiments, the Ig heavy chain may become covalently associated with other Ig heavy/light chain complexes (e.g., by the presence of hinge, CH2, and/or CH3 domains). In the latter case, the heavy/light chain complex that becomes joined to the chimeric construct may constitute an antibody with a specificity distinct from the antibody specificity of the chimeric construct. In some embodiments, the entire chain may be used. In some embodiments, a truncated chain may be used, where all or a part of the CH1, CH2, or CH3 domains may be removed or all or part of the hinge region may be removed. Non-limiting examples of antigen-binding polypeptides include antibodies and antibody fragments such as e.g., murine antibodies, rabbit antibodies, human antibodies, fully humanized antibodies, single chain variable fragments (scFv), single chain antibodies, Fab fragments, F(ab′) fragments, disulfide-linked Fvs (sdFv), intrabodies, minibodies, or diabodies, camelid antibody variable domains and humanized versions, shark antibody variable domains and humanized versions, single domain antibody variable domains, nanobodies (VHHs), and camelized antibody variable domains. In some embodiments, the antigen-binding polypeptide include an scFv.

In some embodiments, the extracellular antigen-binding domain is specific for HER2, or a fragment or variant thereof. In some embodiments, the extracellular antigen-binding domain is specific for IL13Rα2, or a fragment or variant thereof. In some embodiments, the extracellular antigen-binding domain is specific for EphA2, or a fragment or variant thereof.

In some embodiments, the extracellular antigen-binding domain comprises an antibody or antibody fragment.

In a specific embodiment, the extracellular antigen-binding domain comprises an scFv capable of binding to HER2. The scFv capable of binding to HER2 may comprise the amino acid sequence of SEQ ID NO: 17, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 17. In some embodiments, the nucleotide sequence encoding the anti-HER2 scFV comprises the nucleotide sequence that encodes the amino acid sequence of SEQ ID NO: 17, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 17. In some embodiments, the nucleotide sequence encoding the anti-HER2 scFV comprises the sequence set forth in SEQ ID NO: 18, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 18. In some embodiments, the anti-HER2 scFV comprises the amino acid sequence of SEQ ID NO: 17. In some embodiments, the nucleotide sequence encoding the anti-HER2 scFV comprises the nucleotide sequence set forth in SEQ ID NO: 18.

In a specific embodiment, the extracellular antigen binding domain comprises an scFv capable of binding to IL13Rα2. The scFv capable of binding to IL13Rα2 may comprise the amino acid sequence of SEQ ID NO: 29, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 29. In some embodiments, the nucleotide sequence encoding the anti-IL13Rα2 scFV comprises the nucleotide sequence that encodes the amino acid sequence of SEQ ID NO: 29, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 29. In some embodiments, the nucleotide sequence encoding the anti-IL13Rα2 scFV comprises the sequence set forth in SEQ ID NO: 30, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 30. In some embodiments, the anti-IL13Rα2 scFV comprises the amino acid sequence of SEQ ID NO: 29. In some embodiments, the nucleotide sequence encoding the anti-IL13Rα2 scFV comprises the nucleotide sequence set forth in SEQ ID NO: 30.

In a specific embodiment, the extracellular antigen binding domain comprises an scFv capable of binding to EphA2. The scFv capable of binding to EphA2 comprises the amino acid sequence of SEQ ID NO: 38, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 38. In some embodiments, the nucleotide sequence encoding the anti-EphA2 scFV comprises the nucleotide sequence that encodes the amino acid sequence of SEQ ID NO: 38, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 38. In some embodiments, the nucleotide sequence encoding the anti-EphA2 scFV comprises the sequence set forth in SEQ ID NO: 39, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 39. In some embodiments, the anti-EphA2 scFV comprises the amino acid sequence of SEQ ID NO: 38. In some embodiments, the nucleotide sequence encoding the anti-EphA2 scFV comprises the nucleotide sequence set forth in SEQ ID NO: 39.

In some embodiments, the extracellular antigen-binding moiety comprises an antibody or an antibody fragment that binds to an antigen. Antigen-binding moieties may comprise antibodies and/or antibody fragments such as monoclonal antibodies, multispecific antibodies, chimeric antibodies, single-chain Fvs (scFv), single chain antibodies, Fab fragments, F(ab′) fragments, disulfide-linked Fvs (sdFv), intrabodies, minibodies, single domain antibody variable domains, nanobodies (VHHs), diabodies and anti-idiotypic (anti-Id) antibodies (including, e.g., anti-Id antibodies to antigen specific TCR), and epitope-binding fragments of any of the above. Antibodies and/or antibody fragments may be derived from murine antibodies, rabbit antibodies, human antibodies, fully humanized antibodies, camelid antibody variable domains and humanized versions, shark antibody variable domains and humanized versions, and camelized antibody variable domains.

In some embodiments the extracellular antigen-binding moiety comprises an scFv capable of binding to, e.g., CD19, CD22, CD123, CD33, B7-H3 (CD276), HER2, IL13Rα2, and/or EphA2.

In some embodiments, the antigen-binding moiety comprises a ligand. Non-limiting examples of CARs comprising an antigen-binding moiety comprising a ligand include IL-13 mutein-CARs or CD27-CARs. In some embodiments, the antigen-binding moiety may comprise a peptide sequence. Non-limiting examples of CARs comprising an antigen-binding domain comprising a peptide sequence include chlorotoxin and GRP78-CARs. See, for example, PCT Patent Application WO/2021/216994, which is herein incorporated by reference in its entirety.

In some embodiments, the antigen-binding moiety binds to at least one tumor antigen. In some embodiments, the antigen-binding moiety binds to two or more tumor antigens. In some embodiments, the two or more tumor antigens are associated with the same tumor. In some embodiments, the two or more tumor antigens are associated with different tumors.

In some embodiments, the antigen-binding moiety binds to at least one antigen of extracellular matrix. In some embodiments, the antigen-binding moiety binds to two or more antigens of the extracellular matrix. In some embodiments, the two or more tumor antigens are associated with the same extracellular matrix. In some embodiments, the two or more tumor antigens are associated with different extracellular matrix.

In some embodiments, the antigen-binding moiety binds to at least one antigen present on cells within the tumor microenvironment. In some embodiments, the antigen-binding moiety binds to two or more antigens present on cells within the tumor microenvironment. In some embodiments, the two or more antigens are associated with the same cell. In some embodiments, the two or more tumor antigens are associated with different cells.

In some embodiments, the antigen-binding moiety binds to at least one autoimmune antigen. In some embodiments, the antigen-moiety domain binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the antigen-binding moiety binds to at least one infectious antigen. In some embodiments, the antigen-binding moiety binds to two or more infectious antigens. In some embodiments, the two or more infectious antigens are associated with the same infectious disease. In some embodiments, the two or more infectious antigens are associated with different infectious diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of the tumor antigens associated with cervical cancer or head and neck cancer include MUC1, Mesothelin, HER2, GD2, and EGFR. Non-limiting examples of tumor antigens associated with ovarian cancer include FOLR1, FSHR, MUC16, MUC1, Mesothelin, CA125, EpCAM, EGFR, PDGFRa, Nectin-4, B7-H3 and B7-H4. Non-limiting examples of tumor antigens associated with hematological malignancies include BCMA, GPRC5D, SLAM F7, CD33, CD19, CD22, CD79, CLL1, CD123, and CD70. Non-limiting examples of tumor antigens associated with bladder cancer include Nectin-4 and SLITRK6. Non-limiting examples of tumor antigens associated with renal cancer include CD70 and FOLR1. Non-limiting examples of tumor antigen associated with glioblastoma include FGFR1, FGFR3, MET, CD70, ROBO1, IL13Rα2, HER2, EGFRvIII, EGFR, CD133, and PDGFRA. Non-limiting examples of tumor antigen associated with liver cancer include, EpCAM, cMET, AFP, Claudin 18.2, and GPC-3.

Additional examples of antigens that may be targeted by the antigen-binding moiety include, but are not limited to, alpha-fetoprotein, A3, antigen specific for A33 antibody, Ba 733, BrE3-antigen, carbonic anhydrase Ep-CAM, EphAl, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, EX, EGFR, EGP-I, EGP-2, antigen specific for PAM-4 antibody, placental growth factor, p53, prostatic acid phosphatase, PSA, PSMA, RS5, S100, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, TAC, TAG-72, tenascin, VEGF, ED-B fibronectin, COL11A1, 17-IA-antigen, TRAIL receptors, Tn antigen, Thomson-Friedenreich antigens, tumor necrosis antigens, an oncogene marker, an oncogene product, or an angiogenesis marker.

In some embodiments, the antigen is associated with an autoimmune disease or disorder. Such antigens may be derived from cell receptors and cells which produce “self”-directed antibodies. In some embodiments, the antigen is associated with an autoimmune disease or disorder such as, psoriasis, vasculitis, Wegener's granulomatosis, Hashimoto's thyroiditis, Graves' disease, chronic inflammatory demyelinating polyneuropathy, Guillain-Barre syndrome, Crohn's disease, ulcerative colitis, Rheumatoid arthritis (RA), multiple sclerosis (MS), Sjogren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, or Myasthenia gravis.

In some embodiments, autoimmune antigens that may be targeted by the CAR disclosed herein include but are not limited to islet cell antigen, platelet antigens, Sm antigens in snRNPs, myelin protein antigen, Rheumatoid factor, and anticitrullinated protein., glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase fibrinogen, fibrin, vimentin, filaggrin, collagen I and II peptides, alpha-enolase, citrullinated proteins and peptides such as CCP-1, CCP-2 (cyclical citrullinated peptides), translation initiation factor 4G1, perinuclear factor, keratin, Sa (cytoskeletal protein vimentin), circulating serum proteins such as RFs (IgG, IgM), fibrinogen, plasminogen, components of articular cartilage such as collagen II, IX, and XI, ferritin, nuclear components such as RA33/hnRNP A2, Sm, stress proteins such as HSP-65, −70, −90, BiP, inflammatory/immune factors such as B7-H1, IL-1 alpha, and IL-8, enzymes such as calpastatin, alpha-enolase, eukaryotic translation elongation factor 1 alpha laldolase-A, dipeptidyl peptidase, osteopontin, cathepsin G, myeloperoxidase, proteinase 3, antigen, islet cell antigen, rheumatoid factor, histones, ribosomal P proteins platelet antigens, myelin protein, cardiolipin, vimentin, nucleic acids such as, and RNA, ribonuclear particles and proteins such as Sm antigens (including but not limited to SmD's and SmB′/B), U1RNP, A2/B1 hnRNP, Ro (SSA), and La (SSB) antigens, dsDNA, and ssDNA.

In some embodiments, the antigen targeted by CARs of the present disclosure is an antigen expressed in the tumor stroma. Exemplary antigens expressed in the tumor stroma that may be targeted by CARs of the present disclosure include, but are not limited to oncofetal splice variants of fibronectin and tenascin C, tumor-specific splice variants of collagen, and fibroblast activating protein (FAP).

In some embodiments, the antigen targeted by CARs of the present disclosure is an antigens expressed on endothelial cell. Exemplary antigens expressed on endothelial cells that may be targeted by CARs of the present disclosure include, but are not limited to, VEGF receptors, and tumor endothelial markers (TEMs).

Exemplary infectious associated antigens that may be targeted by the modified immune effector cells of the present disclosure include those derived from Adenoviridae (most adenoviruses); Arena viridae (hemorrhagic fever viruses); Birnaviridae; Bungaviridae (e.g., Hantaan viruses, bunga viruses, phleboviruses and Nairo viruses); Calciviridae (e.g., strains that cause gastroenteritis); Coronoviridae (e.g., coronaviruses); Filoviridae (e.g., ebola viruses); Flaviridae (e.g., dengue viruses, encephalitis viruses, yellow fever viruses); Hepadnaviridae (Hepatitis B virus; HBsAg); Herpesviridae (herpes simplex virus (HSV) 1 and 2, varicella zoster virus, cytomegalovirus (CMV), herpes virus); Iridoviridae (e.g., African swine fever virus); Norwalk and related viruses, and astroviruses.; Orthomyxoviridae (e.g., influenza viruses); Papovaviridae (papilloma viruses, polyoma viruses); Paramyxoviridae (e.g., parainfluenza viruses, mumps virus, measles virus, respiratory syncytial virus); Parvovirida (parvoviruses); Picornaviridae (e.g., polio viruses, hepatitis A virus; enteroviruses, human Coxsackie viruses, rhinoviruses, echoviruses); Poxviridae (variola viruses, vaccinia viruses, pox viruses); Reoviridae (e.g., reoviruses, orbiviurses and rotaviruses); Retroviridae (e.g., human immunodeficiency viruses, such as HIV-1 (also referred to as HTLV-III, LAV or HTLV-III/LAV, or HIV-III); and other isolates, such as HIV-LP); Rhabdoviradae (e.g., vesicular stomatitis viruses, rabies viruses); Togaviridae (e.g., equine encephalitis viruses, rubella viruses); and unclassified viruses (e.g., the etiological agents of Spongiform encephalopathies, the agent of delta hepatitis, the agents of non-A, non-B hepatitis (i.e. Hepatitis C)).

Additional infectious antigens that may be targeted by the modified immune effector cells of the present disclosure include bacterial antigens, fungal antigens, parasite antigens, or prion antigens, or the like. Non-limiting examples of infectious bacteria include but are not limited to: Actinomyces israelli, Bacillus antracis, Bacteroides sp., Borelia burgdorferi, Chlamydia., Clostridium perfringens, Clostridium tetani, Corynebacterium diphtheriae, Corynebacterium sp., Enterobacter aerogenes, Enterococcus sp., Erysipelothrix rhusiopathiae, Fusobacterium nucleatum, Haemophilus influenzae, Helicobacter pyloris, Klebsiella pneumoniae, Legionella pneumophilia, Leptospira, Listeria monocytogenes, Mycobacteria sps. (e.g., M tuberculosis, M avium, Mgordonae, M intracellulare, M kansaii), Neisseria gonorrhoeae, Neisseria meningitidis, Pasteurella multocida, pathogenic Campylobacter sp., Rickettsia, Staphylococcus aureus, Streptobacillus monihformis, Streptococcus (anaerobic sps.), Streptococcus (viridans group), Streptococcus agalactiae (Group B Streptococcus), Streptococcus bovis, Streptococcus faecalis, Streptococcus pneumoniae, Streptococcus pyogenes (Group A Streptococcus), Treponema pallidium, and Treponema pertenue. Non-limiting examples of infectious fungi include: Cryptococcus neoformans, Histoplasma capsulatuin, Coccidioides immitis, Blastomyces dernatitidis, Chlamydia trachomatis and Candida albicans. Other infectious organisms (i.e., protists) include: Plasmodium such as Plasmodium falciparum, Plasmodium malariae, Plasmodium ovale, Plasmodium vivax, Toxoplasma gondii and Shistosoma. Other medically relevant microorganisms have been descried extensively in the literature, e.g., see C. G. A. Thomas, “Medical Microbiology”, Bailliere Tindall, Great Britain 1983, which is hereby incorporated by reference in its entirety.

Other examples of antigens that may be targeted by the modified immune cells of the present disclosure include antigens expressed on immune and/or stem cells to deplete these cells such as CD45RA and c-kit.

Various non-limiting exemplary antigen targets are also displayed in Tables 1-3.

In some embodiments, the antigen-binding moiety may comprise a VH sequence, a VL sequence, and/or CDRs thereof, such as those described in the cited publications, the contents of each publication are incorporated herein by reference in their entirety for all purposes (Table 1).

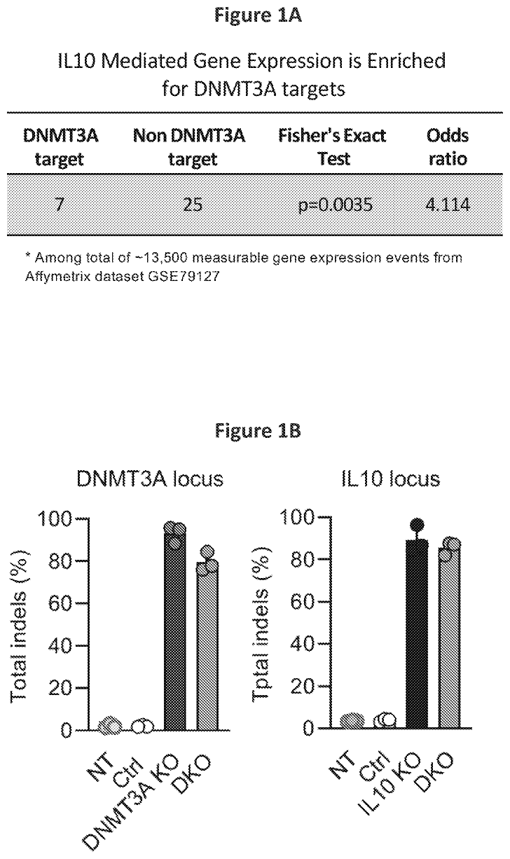

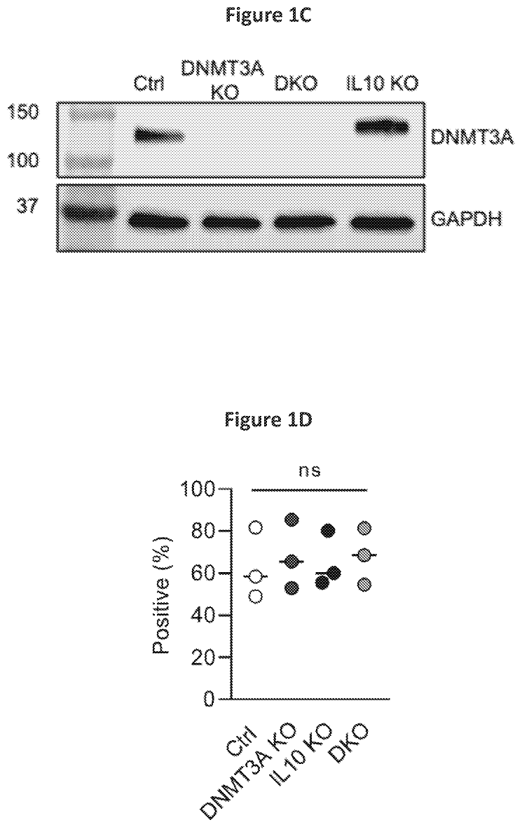

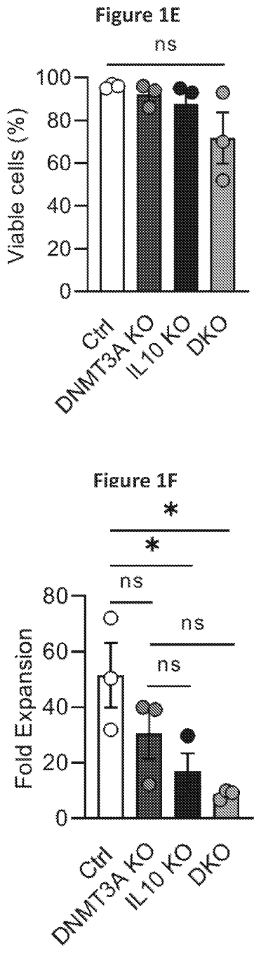

TABLE 1
Exemplary antigen-binding moieties comprising a VH sequence, a VL sequence, and/or CDRs thereof

| Antigen target | Type | Examples of Source | Type | Examples of Source |
|---|---|---|---|---|
| Oncofetal | — | — | VL | Identifier 1, 2, 7 in |
| fibronectin | | | | US20070202103Al |
| VMS2 | VH | FIG. 1 in WO2000058363 | — | — |
| GCC1 | VH | Identifier 1 in US20160030595Al | VL | Identifier 4, 2 in US20160030595A1 |
| MVR | VH | Identifier 1 in US20160257762Al | VL | Identifier 5 in US20160257762A1 |
| OlfmB | VH | Identifier 1 in WO2015054441A1 | VL | Identifier 2 in WO2015054441A1 |
| TRBC1 | VH | Identifier 1 in WO2015132598 | VL | Identifier 2 in WO2015132598 |
| Malignant | VH | Identifier 1 in WO2015133817Al | VL | Identifier 5 in WO2015133817A1 |
| Variable | | | | |
| Receptor | | | | |
| CD40 | VH | Identifier 1 in WO2016069919; | VL | Identifier 2 in WO2016069919; |
| | | Identifier 5, 7, 8 in WO2015091655 | | Identifier 6 in WO2015091655 |
| CDIM | VH | Identifier 1, 10, 11, 12, 13, | VL | Identifier 28, 29, 30, 31, |
| | | 14, 15, 16, 17, 18, 19, 2, 20, | | 32, 33, 34, 35, 36, 37, 38, |
| | | 21, 22, 3, 4, 5, 6, 7, 8, 9 in | | 39, 40, 41, 42, 43, 44, 45, |
| | | WO2013120012 | | 46, 47, 48, 49 in WO2013120012 |
| ILIRAP | VH | Identifier 1, 10, 19, 8, 9 in | VL | Identifier 14, 15, 17, 18, 2, |
| | | WO2016020502; Identifier | | 20 in WO2016020502; |
| | | 120, 122, 124 in WO2016179319A1 | | Identifier 121, 123, 125 in |
| | | | | WO2016179319A1 |
| ALK | VH | Identifier 1, 11, 13, 15, 3, 5, | VL | Identifier 10, 12, 14, 16, 2, |
| | | 7, 9 in US20160280798Al; | | 4, 6, 8 in US20160280798Al; |
| | | Identifier 9, 1, 3, 5, 11, 13, | | Identifier 10, 12, 14, 16, 8 |
| | | 15, 7, 9 in WO2015069922 | | in WO2015069922; |
| | | | | Identifier 2, 4, 6 in WO2015069922 |
| LHR | VH | Identifier 1, 2, 3, 4, 5, 6, 7, 8 | — | — |
| | | in WO2016160618A3 | | |
| MUC16 | VH | Identifier 1, 21, 41, 81, 61 in | VL | Identifier 2, 22, 42, 62, 82 |
| | | WO2016149368; Identifier | | in WO2016149368 |
| | | 11, 4, 6, 8 in US20130171152 | | |
| PTK7 | VH | Identifier 1, 25, 49 in | VL | Identifier 20, 22, 24, 26, |
| | | US20150315293; Identifier | | 28, 30, 32, 34, 36, 38, 40, |
| | | 21, 23, 25, 27, 29, 31, 33, | | 42, 44, 46, 48, 50, 52, 54, |
| | | 35, 37, 39, 41, 43, 45, 47, | | 56, 58, 60, 62, 64, 66, 68 in |
| | | 49, 51, 53, 55, 57, 59, 61, | | WO2012112943A1; |
| | | 63, 65, 67, 69 in WO2012112943A1 | | Identifier 15, 39, 63 in |
| | | | | US20150315293 |
| ANG2 | VH | Identifier 1, 3 in WO2015091655 | VL | Identifier 2, 4 in WO2015091655 |
| CD71 | VH | Identifier 1, 3, 325, 4, 5, 699 | VL | Identifier 2, 327, 329, 331, |
| | | in US20160355599 | | 333, 335, 337, 6, 650, 652, |
| | | | | 654, 656, 658, 660, 670, |
| | | | | 671, 672, 673, 7, 701, 702, |
| | | | | 703, 704, 705, 706, 707, |
| | | | | 708, 709, 710, 711, 712, |
| | | | | 721, 722, 723, 724, 725, |
| | | | | 726, 727, 728, 729, 730, |
| | | | | 731, 732, 733, 734, 735, |
| | | | | 736, 737, 738, 739, 740, |
| | | | | 741, 742, 743, 744, 745, |
| | | | | 746, 747, 748, 749, 750, |
| | | | | 751, 752, 753, 754, 755, |
| | | | | 756, 757, 758, 759, 760, |
| | | | | 761, 762, 763, 764, 765, |
| | | | | 766, 767, 768, 769, 770, |
| | | | | 771, 772, 773, 774, 775, |
| | | | | 776, 777, 778, 779, 780, |
| | | | | 781, 782, 783, 784 785, |
| | | | | 786, 787, 788, 8, 810, 811, |
| | | | | 812, 813, 814, 815, 816, |
| | | | | 817, 818, 819, 820, 821, |
| | | | | 822, 823, 824, 825, 826, |
| | | | | 827, 828, 829, 830, 831, |
| | | | | 832, 833, 834, 835, 836, |
| | | | | 810, 842, 843, 844, 845, |
| | | | | 846, 847, 848, 849, 850, |
| | | | | 851, 852, 853, 854, 855, |
| | | | | 856, 857, 858, 859, 860, |
| | | | | 861, 862, 863, 864, 865, |
| | | | | 866, 867, 868, 869, 870, |
| | | | | 871, 872, 873, 874, 875, |
| | | | | 876, 877, 879, 879, 880, |
| | | | | 881, 882, 883, 884, 885, |
| | | | | 886, 887, 888, 889, 890, |
| | | | | 891, 892, 893, 894, 895, |
| | | | | 896, 897, 898, 899, 900, |
| | | | | 901, 902, 903, 904, 905, |
| | | | | 906, 907, 908 in US20160355599 |
| TEM8 | VH | Identifier 1, 3, 5, 7 in | VL | Identifier 4, 6, 8 in |
| | | US20160264662A1 | | US20160264662A1 |
| IGFI | VH | Identifier 1, 3, 7 in | VL | Identifier 2, 4, 6, 8 in |
| | | WO2007118214; Identifier | | WO2007118214 |
| | | 7 in WO2015073575 | | |
| FAP | VH | Identifier 1, 5 in | VL | Identifier 2, 6 in |
| | | WO2015118030; Identifier | | WO2015118030; Identifier |
| | | 170, 172 in WO2016120216; | | 171, 173 in |
| | | Identifier | | WO2016120216; Identifier |
| | | 8 in US20160326265 A1 | | 9 in US20160326265A1 |
| Mesothelin | VH | Identifier 1, 6 in | VL | Identifier 3, 5 |
| | | WO2015188141; Identifier | | WO2015188141; Identifier |
| | | 119, 50 in US20160333114A1; | | 1, 2, 3 in WO2013142034; |
| | | Identifier 5, 6 in | | Identifier 11, 15, 19, 23, 27 |
| | | WO2013142034; Identifier 15, | | in US20160229919A1; |
| | | 2 in U.S. Pat. No. 9,416,190B2; | | Identifier 120, 47, 49 in |
| | | Identifier 13, 17, 21, 25, 29, | | US20160333114A1 |
| | | 9 in US20160229919A1 | | |
| Frizzled | VH | Identifier 10 in WO2010037041 | VL | Identifier 12, 14 in WO2010037041 |
| Receptor | | | | |
| APCDD1 | VH | Identifier 10, 102, 106, 110, | VL | Identifier 136, 100, 104, |
| | | 114, 118, 122, 126, 130, | | 108, 112, 116, 12, 120, |
| | | 134, 14, 6, 98 in WO2012019061 | | 124, 128, 132, 16, 8 in |
| | | | | WO2012019061 |
| CSF | VH | Identifier 10, 102, 14, 18, 2, | VL | Identifier 12, 32, 44, 48, 60 |
| | | 22, 26, 30, 34, 38, 46, 50, | | in US20050059113Al |
| | | 54, 58, 6, 62, 66, 70, 74, 78, | | |
| | | 82, 86, 90, 94, 98 in | | |
| | | US20050059113A1 | | |
| TIGIT | VH | Identifier 10, 11, 12, 124, | VL | Identifier 130, 131, 132, |
| | | 125, 126, 127, 128, 129, 13, | | 133, 137, 139, 145, 146, |
| | | 136, 138, 14, 143, 144, 149, | | 151, 152, 25, 26, 27, 28, |
| | | 15, 150, 16, 17, 18, 19, 20, | | 29, 30, 50, 51, 52, 64, 95, 8 |
| | | 21, 22, 23, 24, 37, 38, 39, | | in US20160355589 |
| | | 40, 41, 42, 43, 44, 45, 46, | | |
| | | 47, 63, 94, 7, 9 in US20160355589 | | |
| B7H3 | VH | Identifier 10, 11, 12, 13, 14, | VL | Identifier 1, 2, 3, 4, 5, 6, 7, |
| | | 15, 16, 9 in WO2016033225 | | 8 in WO2016033225 |
| IL4R | VH | Identifier 10, 11, 14, 15, 9 in | VL | Identifier 13, 7, 8 in |
| | | WO2009121847 | | WO2009121847 |
| Lymphotoxin | VH | Identifier 10, 12, 14, 16, 2 in | VL | Identifier 1, 15, 4, 6, 8 in |
| beta receptor | | WO2004002431 | | WO2004002431 |
| LGR5 | VH | Identifier 10, 12, 16, 18, 20, | VL | Identifier 15, 19, 21, 23, |
| | | 22, 24, 26, 4 in US20160102146 | | 25, 3 in US20160102146 |
| CD148 | VH | Identifier 10, 14, 18, 2, 22, | VL | Identifier 12, 16, 20, 24, |
| | | 26, 30, 6 in WO2005118643 | | 28, 32, 4, 8 in WO2005118643 |
| GPC3 | VH | Identifier 10, 14, 2, 3, 4, 5, | VL | Identifier 10, 14, 18, 22, |
| | | 6, 7, 8, 9 in US20160208015 Al; | | 24, 26 in US9409994B2; |
| | | Identifier 22 in WO2016049459; | | Identifier 16, 31 in |
| | | Identifier 12, 16, 20, 37, 8 in | | US20160208015Al; |
| | | U.S. Pat. No. 9,409,994B2 | | Identifier 23 in WO2016049459 |
| CSPG4 | VH | Identifier 10, 16, 18, 4, 6, 8 | VL | Identifier 7 in |
| | | in WO2016077638; | | WO2016164429; Identifier |
| | | Identifier 8 in WO2016164429 | | 12, 14 in WO2016077638 |
| AGR2 | VH | Identifier 10, 18 in WO2016040321 | VL | Identifier 11, 19 in WO2016040321 |
| Tissue factor | VH | Identifier 10, 19, 23, 27, 29, | VL | Identifier 25. 31 in |
| | | 6 in WO2004094475; | | US20040229301Al; |
| | | Identifier 38 in US20160333114A1 | | Identifier 12, 21, 25, 31, 8 |
| | | | | in WO2004094475; |
| | | | | Identifier 35, 37 in |
| | | | | US20160333114Al |
| SEMAPHORIN | VH | Identifier 10, 25, 9 in | VL | Identifier 17, 18, 29 in |
| 4D | | US20160115240A1 | | US20160115240Al |
| PDL1 | VH | Identifier 10, 32, 8 in | VL | Identifier 22, 26, 34, 42, |
| | | US20160319022; Identifier | | 58, 66, 74, 82, 86 in |
| | | 18, 30, 38, 46, 50, 54, 62, | | WO2016061142; Identifier 30, 8, |
| | | 70, 78 in WO2016061142; | | 9 in US20150190506; Identifier |
| | | Identifier 29, 7 in | | 7, 9 in US20160319022; |
| | | US20150190506; Identifier | | Identifier 17, 22, 24, 249, |
| | | 16, 18, 197, 247, 248, 250, | | 26, 28, 309, 311, 313, 320, |
| | | 251, 252, 253, 254, 255, | | 325, 34, 340, 357, 359, 36, |
| | | 256, 257, 258, 259, 260, 30, | | 42, 44, 58, 60, 66, 68, 74, |
| | | 308, 310, 312, 319, 32, 324, | | 76, 8, 82, 84, 86, 88 in |
| | | 339, 356, 38, 40, 46, 48, 50, | | US20160108123 |
| | | 52, 54, 6, 62, 70, 72, 78, 80, | | |
| | | 91, 96 in US20160108123; | | |
| | | Identifier 358, 56, 64 in | | |
| | | US20160108123 | | |
| HLAG | VH | Identifier 10, 8 in | VL | Identifier 18, 20 in |
| | | WO2016160622A2 | | WO2016160622A2 |
| B7H4 | VH | Identifier 100, 101, 102, | VL | Identifier 104, 11, 126, |
| | | 103, 107, 108, 109, 110, | | 134, 138, 19, 27, 3, 35, 55, |
| | | 11l, 112, 113, 114, 12, 127, | | 93, 95, 97, 98, 145, 146, |
| | | 130, 131, 132, 133, 137, 2, | | 147, 148 in US20160159910; |
| | | 20, 28, 36, 37, 38, 4, 56, 99, | | Identifier 29, 31, 33 in |
| | | 144 in US20160159910; | | WO2016160620 |
| | | Identifier 13, 15, 17 in | | |
| | | WO2016160620 | | |
| VEGFR2 | VH | Identifier 100, 101, 102, | VL | Identifier 107, 108, 109, |
| | | 103, 114, 115, 116, 117, | | 110, 111, 112, 113, 86, 87, |
| | | 118, 119, 120, 121, 122, | | 88, 89, 90, 91, 92, 93, 94 in |
| | | 123, 124, 95, 96, 97, 98, 99 | | WO2017004254 |
| | | in WO2017004254 | | |
| CD73 | VH | Identifier 100, 103, 107, | VL | Identifier 12. 20, 44, 72, |
| | | 109, 112, 114, 116, 119, | | 76, 8, 84, 92 in |
| | | 121, 16, 32, 4, 52, 60, 68, | | US20160145350; Identifier |
| | | 80, 88 in US20160145350; | | 22, 29, 37, 4 in |
| | | Identifier 135, 40, 21, 3, 28, | | WO2016055609A1; |
| | | 36 in WO2016055609A1 | | Identifier 101, 102, 104, |
| | | | | 106, 110, 117, 118, 120, |
| | | | | 122 in US20160145350 |
| Claudin | VH | Identifier 101, 103, 105, | VL | Identifier 114, 116, 118, |
| | | 107, 109, 111, 113, 115, | | 120, 22, 25, 29, 33, 37, 41, |
| | | 117, 119, 121, 122, 123, | | 45, 49, 53, 57, 61, 65, 69, |
| | | 124, 125, 126, 127, 128, | | 73, 77 in WO2016073649A1 |
| | | 129, 130, 131, 132, 133, | | |
| | | 134, 135, 136, 137, 138, | | |
| | | 139, 140, 141, 142, 143, | | |
| | | 144, 145, 146, 147, 148, | | |
| | | 149, 150, 23, 27, 31, 35, 39, | | |
| | | 43, 47, 51, 55, 59, 63, 67, | | |
| | | 71, 75, 79, 81, 83, 85, 87, | | |
| | | 89, 91, 93, 95, 97, 99 in | | |
| | | WO2016073649A1 | | |
| CLL3 | VH | Identifier 101, 103, 105, | VL | Identifier 100, 102, 104, |
| | | 107, 109, 111, 113, 115, | | 106, 108, 110, 112, 114, |
| | | 117, 119, 121, 123, 125, | | 116, 118, 120, 122, 124, |
| | | 127, 129, 131, 133, 135, | | 126, 128, 130, 132, 134, |
| | | 137, 139, 141, 145, 147, | | 136, 138, 140, 144, 146, |
| | | 149, 151, 153, 155, 157, | | 148, 150, 152, 154, 156, |
| | | 159, 161, 163, 165, 167, | | 158, 160, 162, 164, 166, |
| | | 169, 171, 173, 175, 177, | | 170, 172, 174, 176, 178, |
| | | 179, 181, 183, 185, 187, | | 180, 182, 184, 186, 190, |
| | | 191, 193, 195, 197, 199, | | 192, 194, 196, 198, 20, |
| | | 201, 203, 205, 207, 209, 21, | | 200, 202, 204, 206, 208, |
| | | 211, 213, 23, 25, 27, 29, 31, | | 210, 212, 22, 24, 26, 28, |
| | | 33, 35, 37, 39, 41, 43, 45, | | 30, 32, 34, 36, 38, 40, 42, |
| | | 47, 49, 51, 53, 55, 57, 59, | | 44, 46, 48, 50, 54, 56, 58, |
| | | 61, 63, 65, 67, 69, 71, 73, | | 60, 62, 64, 66, 68, 70, 72, |
| | | 75, 77, 79, 81, 83, 85, 87, | | 74, 76, 78, 80, 82, 84, 86, |
| | | 89, 91, 93, 95, 97, 99 in | | 88, 90, 92, 94, 96, 98 in |
| | | US20170000901 | | US20170000901 |
| OX40 | VH | Identifier 101, 103, 105, | VL | Identifier 10, 45, 47, 49, 8 |
| | | 107, 109, 111, 113, 115, | | in U.S. Pat. No. 8,283,450; |
| | | 117, 119, 121, 123, 124, | | Identifier 11, 7 in |
| | | 125, 17, 28, 318, 37, 48, 50, | | U.S. Pat. No. 9,428,570; |
| | | 58, 66,, 74, 85, 93, 95, 97, | | Identifier 116, 120, 122, |
| | | 99 in WO2016196228; | | 30, 38, 49, 57, 65, 73, 84, |
| | | Identifier 31, 34, 36, 38, 40, | | 86, 94, 98 in WO2016196228; |
| | | 42, 44, 46, 48, 50, 53, 54, | | Identifier 24, 26, 27, 28, |
| | | 55, 58, 59, 61 in | | 30, 60, 8, 81, 82, 83, 84, 85, |
| | | US20150190506; Identifier | | 86, 87, 88, 89 in U.S. Pat. No. |
| | | 33, 35, 37, 39, 41, 43, 45, | | 8,748,585; Identifier 30, 32 in |
| | | 47, 49, 51, 53, 55, 57, 59, | | US20160137740; Identifier |
| | | 61, 63, 65, 67, 71 in | | 32, 35, 39, 41, 43, 45, 47, |
| | | US20160137740; Identifier | | 49, 51, 52, 56, 57, 62 in |
| | | 44, 46, 48, 7, 9 in | | US20150190506; Identifier |
| | | U.S. Pat. No. 8,283,450; | | 29, 37 in US20160137740 |
| | | Identifier 9, 15 in U.S. Pat. | | |
| | | No. 9,428,570; Identifier | | |
| | | 19, 21, 22, 23, 29, 58, 59, | | |
| | | 7, 77, 78, 79, 80 in | | |
| | | U.S. Pat. No. 8,748,585 | | |
| MUCIN1 | VH | Identifier 101, 106, 109, | VL | Identifier 148, 158, 162, |
| | | 115, 119, 123, 127, 141, 15, | | 167, 170, 174, 184, 190, |
| | | 23, 28, 33, 39, 42, 47, 5, 57, | | 193, 203, 208, 211, 220, |
| | | 66, 70, 75, 80, 83, 87, 92 in | | 225, 229, 234, 242, 246, |
| | | EP3049812A2 | | 250, 255, 261, 270, 275, |
| | | | | 279, 283, 291, 297, 303, |
| | | | | 308, 315, 319, 323, 333, |
| | | | | 340 in EP3049812A2 |
| MCSF | VH | Identifier 102, 10, 14, 18, 2, | VL | Identifier 8, 32, 52, 60, 28, |
| | | 22, 26, 30, 34, 38, 46, 50, | | 36, 4, 44, 48, 56, 62, 12, |
| | | 54, 58, 6, 66, 70, 74, 78, 82, | | 16, 20, 24 in WO2005030124 |
| | | 86, 90, 94, 98 in WO2005030124 | | |
| LAG3 | VH | Identifier 102, 106, 110, | VL | Identifier 32, 36, 40, 44, |
| | | 113, 122, 18, 30, 66, 70, 74, | | 48, 52, 56, 60, 84, 88, 92, |
| | | 78 in US20150259420; | | 96, 134, 34, 38, 42, 46, 50, |
| | | Identifier 100, 104, 108, 28, | | 54, 58, 60, 86, 90, 94, 98 in |
| | | 64, 68, 72, 76, 8, 80 in | | US20150259420; Identifier |
| | | US20150259420; Identifier | | 2 in WO2015042246 |
| | | 1 in WO2015042246 | | |
| TIM3 | VH | Identifier 102, 112, 12, 2, | — | — |
| | | 22, 32, 42, 52, 62, 72, 82, 91 | | |
| | | in US20150086574; | | |
| | | Identifier 82 in WO2013006490; | | |
| | | Identifier 13, 21, 29, 37, 45, | | |
| | | 5, 53, 61, 69, 77, 85, 93 in | | |
| | | WO2016179319A1; | | |
| | | Identifier 7 in WO2013006490; | | |
| | | Identifier 107, 117, 17, 27, | | |
| | | 37, 47, 57, 67, 7, 77, 87, 97 | | |
| | | in US20150086574; Identifier | | |
| | | 17, 25, 33, 41, 49, 57, 65, | | |
| | | 73, 81, 89, 9, 97 in | | |
| | | WO2016179319A1 | | |
| CD2 | VH | Identifier 103, 117, 119 in | VL | Identifier 102, 116 in WO2016122701 |
| | | WO2016122701 | | |
| CD52 | VH | Identifier 103, 136, 137 in | VL | Identifier 102, 138 in WO2010132659 |
| | | WO2010132659 | | |
| WT1/HLA | VH | Identifier 104, 111, 128, 14, | VL | Identifier 106, 112, 130, |
| Bispecific | | 32, 50, 68, 86 in WO2015070061 | | 34, 52, 70, 88 in WO2015070061 |
| CD3 | VH | Identifier 108, 112, 115 in | VL | Identifier 104 in WO2016122701; |
| | | WO2016122701; Identifier | | Identifier 13 in WO2016126213A1 |
| | | 29 in WO2014144722 A2; | | |
| | | Identifier 12 in WO2016126213A1 | | |
| PG9 | VH | Identifier 11 in EP3074419A2 | VL | Identifier 10 in EP3074419A2 |
| V2 | VH | Identifier 11 in US20160194375Al | — | — |
| HSP70 | VH | Identifier 11, 12 in WO2016120217 | VL | Identifier 16, 17 in WO2016120217 |
| CD37 | VH | Identifier 11, 12, 18 in | VL | Identifier 14, 15 in US20170000900 |
| | | US20170000900 | | |
| CD123 | VH | Identifier 11, 13, 14, 21 in | VL | Identifier 9, 11, 18, 19, 20, |
| | | WO2015140268A1; | | 21, 22, 23 in WO2016120220; |
| | | Identifier 113, 115, 57, 59, | | Identifier 12, 16, 18, 19, |
| | | 63 in WO2016120216; | | 22 in WO2015140268A1; |
| | | Identifier 12, 123, 24, 25, | | Identifier 275, 276, 277, |
| | | 26, 27, 28, 29, 30, 9 in | | 278, 307, 308, 309, 310 in |
| | | WO2016120220; Identifier | | WO2016028896; Identifier |
| | | 216, 217, 218, 219, 274 in | | 5 in US20160333108A1; |
| | | WO2016028896 | | Identifier 114, 116, 58, 60, |
| | | | | 64 in WO2016120216 |
| GD3 | VH | Identifier 11, 13, 15, 17 in | VL | Identifier 12, 14, 16, 18 in |
| | | WO2016185035A1 | | WO2016185035A1 |
| TAG72 | VH | Identifier 115 in US20160333114A1 | VL | Identifier 116 in US20160333114Al |
| MCAM | VH | Identifier 115, 116, 117, | VL | Identifier 109, 110, 111, |
| | | 118, 119, 157, 158, 159, | | 112, 121, 122, 123 in |
| | | 160, 161, 178, 179 in | | US20150259419; Identifier |
| | | US20150259419; Identifier | | 30, 40, 50, 60, 70, 71, 72 in |
| | | 35, 45, 55, 65, 77, 89 in | | US20150239980 |
| | | US20150239980; Identifier | | |
| | | 101, 102, 103, 104, 105, | | |
| | | 106, 107 in US20150259419 | | |
| CA19.9 | VH | Identifier 117 in US20160333114Al | VL | Identifier 118 in US20160333114A1 |
| BMPR1A | VH | Identifier 12 in WO2011116212 | — | — |
| LGR4 | VH | Identifier 12, 13, 5, 9 in | VL | Identifier 10, 11, 6 in |
| | | US20160046723 | | US20160046723 |
| APRIL | VH | Identifier 12, 14, 16. 18. 3. | VL | Identifier 20, 22, 24, 26, |
| | | 32, 34, 36, 38, 40, 42, 44, | | 28, 30, 4, 50 in US2016026467 |
| | | 46, 48, 52 in US20160264674 | | |
| FcRL5 | VH | Identifier 12, 16, 20, 24, 28, | VL | Identifier 11, 15, 19, 23, |
| (FcReceptorLike 5) | | 32, 36, 4, 40, 44, 48, 8, 915, | | 27, 3, 31, 35, 39, 43, 47, 7, |
| | | 919 in WO2016090337 | | 917, 921 WO2016090337 |
| CLDN18.2 | VH | Identifier 12, 2 in | VL | Identifier 13, 3 in |
| | | US20160347815Al | | US20160347815Al |
| ROR1 | VH | Identifier 12, 20, 28, 36, 44, | VL | Identifier 16, 24, 32, 40, |
| | | 60, 68 WO2016016343A1; | | 56, 64, 72, 36, 62, 23, 49, |
| | | Identifier 57, 19, 31, 45, 53, | | 58 WO2016016343A1; |
| | | 71 in WO2016016344A1; | | Identifier 86, 88, 90 in |
| | | Identifier 85, 87, 89 in | | WO2016120216; Identifier |
| | | WO2016120216; Identifier | | 126, 127, 234, 235, 236, |
| | | 122, 125, 175, 176, 179, | | 237, 238, 240, 241, 242, |
| | | 180, 181, 182, 183, 184, | | 243, 244, 245, 246, 247, |
| | | 185, 186, 187, 188, 189, | | 248 in US20160208018A1; |
| | | 190, 191, 192, 193, 194, | | Identifier 56 in |
| | | 195, 196, 197, 197, 199, | | EP3083671A1; Identifier |
| | | 200, 201, 202, 203, 204, | | 103, 111, 127, 135, 143, |
| | | 205, 206, 207, 208, 209 in | | 15, 151, 159, 167, 175, |
| | | US20160208018A1; | | 183, 191, 199, 207, 215, |
| | | Identifier 55 in | | 223, 23, 231, 239, 247, |
| | | EP3083671Al; Identifier | | 255, 263, 271, 279, 287, |
| | | 104, 112, 120, 128, 152, 16, | | 295, 303, 31, 311, 319, |
| | | 160, 168, 176, 184, 192, | | 327, 335, 343, 351, 359, |
| | | 200, 208, 216, 224, 232, 24, | | 39, 47, 55, 63, 7, 71, 79, |
| | | 240, 248, 256, 264, 272, | | 87, 95 in WO2016187216A1 |
| | | 280, 288, 296, 304, 312, 32, | | |
| | | 320, 336, 344, 352, 360, 40, | | |
| | | 48, 56, 64, 72, 8, 80, 88 in | | |
| | | WO2016187216A1 | | |
| B7H1 | VH | Identifier 12, 32, 42, 52, 72, | VL | Identifier 17, 37, 47, 57, 7, |
| | | 2, 62 in US20130034559 | | 77, 27, 67 in US20130034559 |
| CD32B | VH | Identifier 127 in WO2016122701 | VL | Identifier 126 in WO2016122701 |
| CD64 | VH | Identifier 129 in WO2016122701 | VL | Identifier 128 in WO2016122701 |
| PG16 | VH | Identifier 13 in EP3074419A2 | VL | Identifier 12 in EP3074419A2 |
| MPER | VH | Identifier 13 in US20160194375A1 | VL | Identifier 12 in US20160194375A1 |
| CD105 | VH | Identifier 13, 14, 16 in | VL | Identifier 1, 17, 20, 22, 23 |
| | | WO2014039682 | | in WO2014039682 |
| CLL1 | VH | Identifier 13, 14, 15, 17, 19, | VL | Identifier 16, 18, 20, 22, |
| | | 21, 23, 25, 27, 29, 31, 33, 35 | | 24, 26, 28, 30, 32, 24, 36 in |
| | | in WO2016120219; | | WO2016120219; Identifier |
| | | Identifier 195, 65, 66, 67, | | 196, 78, 79, 80, 81, 82, 83, |
| | | 68, 69, 70, 71, 72, 73, 74, | | 84, 85, 86, 87, 88, 89, 90 in |
| | | 75, 76, 77 in WO2016014535; | | WO2016014535; Identifier |
| | | Identifier 31, 33, 34, 36, 38, | | 30, 32, 35, 37, 39, 41 in |
| | | 40, 42, 46 in US20160075787; | | US20160075787; Identifier |
| | | Identifier 150, 103, 105, | | 152, 104, 106, 108, 110, |
| | | 107, 109, 111, 113, 115, 117 | | 112, 114, 116, 118 in |
| | | in WO2016179319A1 | | WO2016179319A1 |
| GPRC5D | VH | Identifier 13, 17, 21, 25, 29, | VL | Identifier 10, 14, 18, 2, 22, |
| | | 314, 326, 33, 338, 350, 362, | | 26, 30, 303, 315, 327, 339, |
| | | 37, 374, 386, 41, 45, 49, 5, | | 34, 351, 363, 375, 38, 387, |
| | | 53, 57, 61, 65, 69, 73, 77, | | 42, 46, 50, 54, 58, 6, 62, |
| | | 81, 85, 89, 93, 1, 9 in | | 66, 70, 74, 78, 82, 86, 94 in |
| | | WO2016090312 | | WO2016090312 |
| EFNA4 | VH | Identifier 13, 39 in US20150125472 | — | — |
| CD79 | VH | Identifier 131 in WO2016122701 | VL | Identifier 130 in WO2016122701 |
| FGFR3 | VH | Identifier 132, 134, 136 in | VL | Identifier 133, 135, 137, |
| | | U.S. Pat. No. 9,499,623 | | 139 in U.S. Pat. No. 9,499,623 |
| TCR | VH | Identifier 133 in WO2016122701 | VL | Identifier 132 in WO2016122701 |
| MN | VH | Identifier 133, 135, 137, | VL | Identifier 134, 136, 138, |
| | | 139, 141, 143, 145, 147, | | 140, 142, 144, 146, 148, |
| | | 149, 151 in WO2007070538 | | 150, 152 in WO2007070538 |
| IL33 | VH | Identifier 134, 136, 138, | VL | Identifier 135, 137, 139, |
| | | 185, 187, 189, 216, 218, | | 184, 188, 217, 219, 237, |
| | | 220, 221,236, 246, 282, 284, | | 247, 283, 285, 287, 37, 39, |
| | | 286, 36, 38, 40, 84, 86, 88 | | 41, 87 in US20160168242 |
| | | in US20160168242 | | |
| NKG2D | VH | Identifier 135, 137 in WO2016122701 | VL | Identifier 134, 136 in WO2016122701 |
| CD30 | VH | Identifier 14, 16 in WO2016134284 | VL | Identifier 13, 15 in WO2016134284 |
| EGFR | VH | Identifier 14, 50, 9 in | VL | Identifier 15 in |
| | | WO2015143382; Identifier | | WO2015143382; Identifier |
| | | 12, 14, 15, 21 in | | 14 in WO2014143765; |
| | | US20100008978Al; | | Identifier 4051, 4052, |
| | | Identifier 2123 in | | 4053, 4054, 4055, 4056, |
| | | WO2018231759 | | 4057, 4058, 4059, 4060, |
| | | | | 4061, 4062, 4063, 4064, |
| | | | | 4065, 4066, 4067, 4068, |
| | | | | 4069, 4070, 4071, 4072, |
| | | | | 4073, 4074, 4075, 4076, |
| | | | | 4077, 4078, 4079, 4080, |
| | | | | 4081, 4082, 4083, 4084, |
| | | | | 4085, 4086, 4087, 4088, |
| | | | | 4089. 4090, 4091, 4092, |
| | | | | 4093, 4094, 4095, 4096, |
| | | | | 4097, 4098, 4099, 4100, |
| | | | | 4101,4102, 4103, 4104, |
| | | | | 4105, 4106, 4107, 4108, |
| | | | | 4109, 4110, 4111, 4112, |
| | | | | 4113, 4114, 4115, 4116, |
| | | | | 4117, 4118, 4119, 4120, |
| | | | | 4121, 4122, 4123, 4124, |
| | | | | 4125, 4126, 4127, 4128, |
| | | | | 4129, 4130, 4131, 4132, |
| | | | | 4133, 4134, 4135, 4136, |
| | | | | 4137, 4138, 4139, 4140, |
| | | | | 4141, 4142, 4143, 4144, |
| | | | | 4145, 4146, 4147, 4148, |
| | | | | 4149, 4150, 4151, 4152, |
| | | | | 4153, 4154, 4155, 4156, |
| | | | | 4157, 4158, 4159, 4160, |
| | | | | 4161, 4162, 4163, 4164, |
| | | | | 4165, 4166, 4167, 4168, |
| | | | | 4169, 4170, 4171, 4172, |
| | | | | 4173, 4174, 4175, 4176, |
| | | | | 4177, 4178, 4179, 4180, |
| | | | | 4181, 4182, 4183, 4184, |
| | | | | 4185, 4186, 4187, 4188, |
| | | | | 4189, 4190, 4191, 4192, |
| | | | | 4193, 4194, 4195, 4196, |
| | | | | 4197, 4198, 4199, 4200, |
| | | | | 4201, 4202, 4203, 4204, |
| | | | | 4205, , 4206, 4207, 4208, |
| | | | | 4209, 4210, 4211, 4212, |
| | | | | 4213, 4214, 4215, 4216, |
| | | | | 4217, 4218, 4219, 4220, |
| | | | | 4221, 4222, 4223, 4224, |
| | | | | 4225, 4226, 4227, 4228, |
| | | | | 4229, 4230, 4231, 4232, |
| | | | | 4233, 4234, 4235, 4236, |
| | | | | 4237, 4238, 4239, 4240, |
| | | | | 4241, 4242, 4243, 4244, |
| | | | | 4245, 4246, 4247, 4248, |
| | | | | 4249, 4250, 4251, 4252, |
| | | | | 4253, 4254, 4255, 4256, |
| | | | | 4257, 4258, 4259, 4260, |
| | | | | 4261, 4262, 4263, 4264, |
| | | | | 4265, 4266, 4267, 4268, |
| | | | | 4269, 4270, 4271, 4272, |
| | | | | 4273, 4274, 4275, 4276, |
| | | | | 4277, 4278, 4279, 42870, |
| | | | | 4281, 4282, 42834284, |
| | | | | 4285, 4286, 4287, 4288, |
| | | | | 4289, 4290, 4291, 4292, |
| | | | | 4293, 4294, 4295, 4296, |
| | | | | 4297, 4298, 4299, 4300, |
| | | | | 4301, 4302, 4303, 4304, |
| | | | | 4305, 4306, 4307, 4308, |
| | | | | 4309, 4310, 4311, 4312, |
| | | | | 4313, 4314, 4315, 4316, |
| | | | | 4317, 4318, 4319, 4320, |
| | | | | 4321, 4322, 4323, 4324, |
| | | | | 4325, 4326, 4327, 4328, |
| | | | | 4329, 4330, 4331, 4332, |
| | | | | 4333, 4334, 4335, 4336, |
| | | | | 4337, 4338, 4339, 4340, |
| | | | | 4341, 4342, 4343, 4344, |
| | | | | 4345, 4346, 4347, 4348, |
| | | | | 4349, 4350, 4351, 4352, |
| | | | | 4353, 4354, 4355, 4356, |
| | | | | 4357, 4358, 4359, 4360, |
| | | | | 4361, 4362, 4363, 4364, |
| | | | | 4365, 4366, 4367, 4368, |
| | | | | 4369, 4370, 4371, 4372, |
| | | | | 4373, 4374, 4375, 4376, |
| | | | | 4377, 4378, 4379, 4380, |
| | | | | 4381, 4382, 4383, 4384, |
| | | | | 4385, 4386, 4387, 4388, |
| | | | | 4389, 4390, 4391, 4392, |
| | | | | 4393, 4394, 4395, 4396, |
| | | | | 4397, 4398, 4399, 4400, |
| | | | | 4401, 4402, 4403, 4404, |
| | | | | 4405, 4406, 4407, 4408, |
| | | | | 4409, 4410, 4411, 4412, |
| | | | | 4413, 4414, 4415, 4416, |
| | | | | 4417, 4418, 4419, 4420, |
| | | | | 4421, 4422, 4423, 4424, |
| | | | | 4425, 4426, 4427, 4428, |
| | | | | 4429, 4430, 4431, 4432, |
| | | | | 4433, 4434, 4435, 4436, |
| | | | | 4437, 4438, 4439, 4440, |
| | | | | 4441, 4442, 4443, 4444, |
| | | | | 4445, 4446, 4447, 4448, |
| | | | | 4449, 4450, 4451, 4452, |
| | | | | 4453, 4454, 4455, 4456, |
| | | | | 4457, 4458, 4459, 4460, |
| | | | | 4461, 4462, 4463, 4464, |
| | | | | 4465, 4466, 4467, 4468, |
| | | | | 4469, 4470, 4471, 4472, |
| | | | | 4473, 4474, 4475, 4476, |
| | | | | 4477, 4478, 4479, 4480, |
| | | | | 4481, 4482, 4483, 4484, |
| | | | | 4485, 4486, 4487, 4488, |
| | | | | 4489, 4490, 4491, 4492, |
| | | | | 4493, 4494, 4495, 4496, |
| | | | | 4497, 4498, 4499, 4500, |
| | | | | 4501, 4502, 4503, 4504, |
| | | | | 4505, 4506, 4507, 4508, |
| | | | | 4509, 4510, 4511, 4512, |
| | | | | 4513, 4514, 4515, 4516, |
| | | | | 4517, 4518, 4519, 4520, |
| | | | | 4521, 4522, 4523, 4524, |
| | | | | 4525, 4526, 4527, 4528, |
| | | | | 4529, 4530, 4531, 4532, |
| | | | | 4533, 4534, 4535, 4536, |
| | | | | 4537, 4538, 4539, 4540, |
| | | | | 4541, 4542, 4543, 4544, |
| | | | | 4545, 4546, 4547, 4548, |
| | | | | 4549, 4550, 4551, 4552, |
| | | | | 4553, 4554, 4555, 4556, |
| | | | | 4557, 4558, 4559, 4560, |
| | | | | 4561, 4562, 4563, 4564, |
| | | | | 4565, 4566, 4567, 4568, |
| | | | | 4569, 4570, 4571, 4572, |
| | | | | 4573, 4574, 4575, 4576, |
| | | | | 4577, 4578, 4579, 4580, |
| | | | | 4581, 4582, 4583, 4584, |
| | | | | 4585, 4586, 4587, 4588, |
| | | | | 4589, 4590, 4591, 4592, |
| | | | | 4593, 4594, 4595, 4596, |
| | | | | 4597, 4598, 4599, 4600, |
| | | | | 4601, 4602, 4603, 4604, |
| | | | | 4605, 4606, 4607, 4608, |
| | | | | 4609, 4610, 4611, 4612, |
| | | | | 4613, 4614, 4615, 4616, |
| | | | | 4617, 4618, 4619, 4620, |
| | | | | 4621, 4622, 4623, 4624, |
| | | | | 4625, 4626, 4627, 4629, |
| | | | | 4629, 4630, 4631, 4632, |
| | | | | 4633, 4634, 4635, 4636, |
| | | | | 4637, 4638, 4639 in |
| | | | | WO2018231759 |
| Her2 | VH | Identifier 141, 262, 264, | VL | Identifier 140 in WO2016054555A2; |
| | | 266, 268, 270 in WO2016168773A3; | | Identifier 261, 263, 265, |
| | | Identifier 11 in U.S. Pat. No. | | 267, 269 in WO2016168773A3; |
| | | 9,518,118; | | Identifier 10, 18, 23 in |
| | | Identifier 62 in US20160333114Al; | | U.S. Pat. No. 9,518,118; |
| | | Identifier 19, 24 in | | Identifier 3 in WO2016168769A1; |
| | | U.S. Pat. No. 9,518,118 | | Identifier 59, 61 in |
| | | | | US20160333114Al |
| EFNA | VH | Identifier 149, 153, 157, 161 | VL | Identifier 151, 155, 159, |
| | | in US20150125472 | | 163 in WO2012118547; |
| | | | | Identifier 27, 53 in |
| | | | | US20150125472 |
| PGT1 | VH | Identifier 15 in EP307441 | — | — |
| Factor XII | VH | Identifier 15 in WO2014089493 | VL | Identifier 17 in WO2014089493 |
| E7MC | VH | Identifier 15, 16, 17, 18, 19, | VL | Identifier 238, 239, 240, |
| | | 20, 21, 22, 23, 233, 234, | | 241, 242, 243, 36, 37, 38, |
| | | 235, 236, 237, 24, 25, 26, | | 39, 41, 42, 43, 44, 45, 46, |
| | | 27, 28, 29, 30, 31, 32, 33, | | 47, 48, 49, 50, 51, 52, 53, |
| | | 34, 35 in WO2016182957A1 | | 54, 55, 56 in WO2016182957A1 |
| ICOS | VH | Identifier 15, 16, 19, 23, 7 in | VL | Identifier 17, 18, 20, 24, 8 |
| | | US20160215059 | | in US20160215059 |
| CD76b | VH | Identifier 15, 17, 19, 23, 27, | VL | Identifier 16, 18, 22, 38, |
| | | 29, 37, 57, 59, 61 in | | 58, 60, 62 in US20160159906 |
| | | US20160159906 | | |
| MUC1C ECD | VH | Identifier 15, 19, 23, 60, 64, | VL | Identifier 17, 21, 25, 62, |
| | | 68, 72 in US20160340442A1 | | 66, 70, 75 in US20160340442Al |
| CD4BS | VH | Identifier 15, 3 in | VL | Identifier 14, 2 in US20160194375A1 |
| | | US20160194375A1 | | |
| CD7 | VH | Identifier 16, 20 in | VL | Identifier 17, 21 in WO2016126213A1 |
| | | WO2016126213A1 | | |
| PGT2 | VH | Identifier 17 in EP3074419A2 | VL | Identifier 16 in EP3074419A2 |
| OTK3 | VH | Identifier 17 in WO2015158868 | VL | Identifier 18 in WO2015158868 |
| GD2 | VH | Identifier 17 in WO2016134284; | VL | Identifier 10, 2, 5, 7, 9 in |
| | | Identifier 1 in US20130216528; | | US20130216528; Identifier |
| | | Identifier 10, 9 in | | 11, 12 in WO2015132604; |
| | | WO2015132604; Identifier | | Identifier 18 in WO2016134284 |
| | | 3, 4, 6, 8 in US20130216528 | | |
| Trophoblast | VH | Identifier 17, 13, 15, 11 in | VL | Identifier 18, 12, 14, 16 in |
| Glycoprotein | | WO2016034666A1 | | WO2016034666A1 |
| 5T4 | | | | |
| AMC | VH | Identifier 17, 18, 19, 20, 21, | VL | Identifier 27, 28, 29, 31, 32, |
| | | 22, 23, 24, 25, 26 in WO2016161390 | | 33, 34, 35, 36 in WO2016161390 |
| Factor D | VH | Identifier 17, 20, 27, 29, 30, | VL | Identifier 16, 18, 19, 26, 3 |
| | | 31, 32, 33, 4 in US20160017052 | | in US20160017052 |
| CD276 | VH | Identifier 17, 26, 7 in | VL | Identifier 18, 27 in |
| | | US20160053017 | | US20160053017 |
| B7H3 (CD276) | VH | Identifier 17, 26, 7 in | VL | Identifier 18, 27, 8 in |
| | | WO2016044383 | | WO2016044383 |
| RAS | VH | Identifier 17, 47, 57, 67, 7, | VL | Identifier 19, 49, 59, 69, |
| | | 77 in WO2016154047 | | 79, 9 in WO2016154047 |
| DR5 | VH | Identifier 18, 82, 90, 98, 8 in | VL | Identifier 13, 23, 25, 27, 3, |
| | | WO2016122701 | | 78, 86, 94, 29 in WO2016122701; |
| | | | | Identifier 62 in WO2016122701; |
| | | | | Identifier 54 in WO2016122701; |
| | | | | Identifier 70 in WO2016122701 |
| PGT3 | VH | Identifier 19 in EP3074419A2 | VL | Identifier 18 in EP3074419A2 |
| PD1 | VH | Identifier 19 in US20150290316; | VL | Identifier 2, 39, 7, 8, 9 in |
| | | Identifier 25, 26, 27, 28, 29 in | | US 20160159905; |
| | | US20130291136; Identifier 29, 3, | | Identifier 21 in US20150290316; |
| | | 38 in US 20160159905; | | Identifier 30, 31, 32, 33 in |
| | | Identifier 38, 50 in WO2015112900; | | US20130291136; Identifier |
| | | Identifier 4, 4, 6 in US | | 42, 46, 54 in WO2015112900; |
| | | 20160159905; Identifier 82, | | Identifier 58, 62, 66, 70, 74, |
| | | 86 in WO2015112900; | | 78 in WO2015112900; |
| | | Identifier 17 in WO2014055648; | | Identifier 18 in WO2014055648; |
| | | Identifier 4 in WO2016040892; | | Identifier 5 in WO2016040892; |
| | | Identifier 12 in US20150190506 | | Identifier 13 in US20150190506 |
| NYBR1 | VH | Identifier 19 in US20160333422A1 | VL | Identifier 18 in US20160333422A1 |
| CD28 | VH | Identifier 19 in WO2015158868 | VL | Identifier 20 in WO2015158868 |
| Tn Glycopeptide | VH | Identifier 19, 20 in WO2015120180 | — | — |
| humanERBB3 | VH | Identifier 19, 29, 38, 45, 55, | VL | Identifier 10, 20, 30, 39, |
| | | 61, 9 in WO2013052745 | | 46, 56, 62 in WO2013052745 |
| Olfml3 | VH | Identifier 19, 3 in WO2015054441A1 | VL | Identifier 20, 4 in WO2015054441A1 |
| RHAMM | VH | Identifier 2 in WO2000029447 | VL | Identifier 4 in WO2000029447 |
| antagonist body | | | | |
| CD38 | VH | Identifier 2 in WO2009080830; | VL | Identifier 1, 11 in WO2009080830 |
| | | Identifier 10 in WO2015121454 | | |
| PDl (Nivolumab) | VH | Identifier 2 in WO2016040892; | VL | Identifier 11 in US20150190506 |
| | | Identifier 10 in US20150190506 | | |
| PDK1 | VH | Identifier 2 in WO2016090365 | VL | Identifier 9 in WO2016090365 |
| IL21 | VH | Identifier 2, 3 in US20160145332 | — | — |
| ERBB2 | VH | Identifier 2, 4 in US20110129464; | VL | Identifier 1 in US20110129464; |
| | | Identifier 10, 2, 26, 30, 38, 4, | | Identifier 12, 16, 20, 24, 32, 36, |
| | | 40, 42, 52, 54, 56, 57, 58, 6 in | | 44, 50, 51, 53, 8 in US20130089544; |
| | | US20130089544; Identifier | | Identifier 7 in US20130266564; |
| | | 8 in US20130266564; | | Identifier 3 in US20110129464 |
| | | Identifier 1 in US20150104443 | | |
| 5T4 | VH | Identifier 2, 4 in WO2016022939 | VL | Identifier 1, 3 in WO2016022939 |
| GPDL1 | VH | Identifier 20 in US20160108123 | — | — |
| GM2 | VH | Identifier 20, 22, 23, 26, 27, | VL | Identifier 21, 24, 25, 31, |
| | | 28, 29, 30 in US20090028877 | | 32, 33, 34, 35 in US20090028877 |
| EphA2 receptor | VH | Identifier 20, 22, 24, 32, 34, | VL | Identifier 26, 28, 30, 47, |
| | | 36, 37, 38, 40, 42, 43, 45, | | 48, 49, 50, 52, 78, 80 in |
| | | 74, 76 in US20150274824 | | US20150274824 |
| KDR | VH | Identifier 20, 24, 26, 29, 31, | VL | Identifier 22 in WO2003075840 |
| | | 33 in WO2003075840 | | |
| PGT4 | VH | Identifier 21 in EP3074419A2 | VL | Identifier 20 in EP3074419A2 |
| CD324 | VH | Identifier 21, 23, 25, 27, 29, | VL | Identifier 20, 22, 24, 26, |
| | | 31, 33, 35, 37, 39, 41, 43, | | 28, 30, 32, 34, 36, 38, 40, |
| | | 45, 47, 49, 51, 53, 55, 57, | | 42, 44, 46, 48, 50, 52, 54, |
| | | 59, 61, 63, 65, 67, 69, 71 in | | 56, 58, 60, 62, 64, 66, 68, |
| | | U.S. Pat. No. 9,534,058 | | 70 in U.S. Pat. No. 9,534,058 |
| AXL | VH | Identifier 21, 3, 45 in | VL | Identifier 22, 4 in |
| | | WO2016097370 | | WO2016097370 |
| collagen | VH | Identifier 21, 4, 15, 17, 18, | VL | Identifier 11, 12, 14, 23, |
| | | 19, 20, 5, 6, 7, 1, 2, 3 in | | 25, 26, 27, 8, 9 in WO2007024921 |
| | | WO2007024921 | | |
| EGFR | VH | Identifier 2124 in WO2018231759; | VL | 4640, 4641, 4642, 4643, 4644, 4645, |
| (EGFRvIII) | | Identifier 13 in WO2016016341; | | 4646, 4647, 4648, 4649, 4650, 4651, |
| | | Identifier 24 in WO2016168773 A3; | | 4652, 4653, 4654, 4655, 4656, 4657, |
| | | Identifier 34 in US20160304615; | | 4658 in WO2018231759; |
| | | Identifier 2 in US20160200819A1; | | Identifier 14 in WO2016016341; |
| | | Identifier 91, 93 in WO2016120216 | | Identifier 23 in WO2016168773 A3; |
| | | | | Identifier 42 in US20160304615; |
| | | | | Identifier 1 in US20160200819A1; |
| | | | | Identifier 92, 94 in WO2016120216 |
| IL3alpha | VH | Identifier 22 in WO2008127735 | VL | Identifier 27, 37 in WO2008127735 |
| CS1 | VH | Identifier 22 in WO2016168773 A3; | VL | Identifier 104, 106, 108 in |
| | | 103, 105, 107, 109 in WO2016120216; | | WO2016120216; Identifier 14, 16, |
| | | Identifier 13, 15, 17, 19 in | | 18, 20, 22 in WO2015166056A1; |
| | | WO2015166056A1; | | Identifier 39, 41, 43, 45, |
| | | Identifier 38, 40, 42, 44, | | 47 in WO2015121454; |
| | | 46 in WO2015121454; | | Identifier 110, 112 in WO2016120216 |
| | | Identifier 26 in US20160075784Al | | |
| KMA | VH | Identifier 22 in WO2016172703 A2 | VL | Identifier 2, 21 in WO2016172703A2 |
| PGT5 | VH | Identifier 23 in EP3074419A2 | VL | Identifier 22 in EP3074419A2 |
| CD45 | VH | Identifier 24 in WO2016126213A1 | VL | Identifier 25 in WO2016126213A1 |
| leukocytegenA2 | VH | Identifier 25 in WO2010065962 A2 | — | — |
| CD16 | VH | Identifier 25 in WO2015158868 | VL | Identifier 26 in WO2015158868 |
| BCMA | VH | Identifier 26 in WO2016168773 A3; | VL | Identifier 25 in WO2016168773 A3; |
| | | Identifier 142, 148, 154, | | Identifier 42 in WO2016097231; |
| | | 160, 166, 172, 178, 184, | | Identifier 143, 149, 155, 161, |
| | | 190, 196, 202, 208, 214, | | 167, 173, 179, 185, 191, 197, |
| | | 220, 226, 232, 238, 244, | | 203, 209, 215, 221, 227, |
| | | 250, 256, 262, 268, 274, | | 233, 239, 245, 251, 257, |
| | | 280, 286, 292, 298, 304, | | 263, 269, 275, 281, 287, |
| | | 310, 316, 322, 328, 334, | | 293, 299, 305, 311, 317, |
| | | 340, 346, 352 in WO2016168595A1; | | 323, 329, 335, 341, 347, |
| | | Identifier 8 in WO2016094304A3; | | 353 in WO2016168595 Al; |
| | | Identifier 171, 172, 173, | | Identifier 192, 193, 194, |
| | | 174, 175, 176, 177, 178, | | 195, 196, 197, 198, 199, |
| | | 179, 180, 181, 182, 183, | | 200, 201, 204, 205, 207, |
| | | 184, 185, 186, 187, 190, | | 208, 211, 259, 260, 84, 85, |
| | | 255, 257, 258, 69, 70, 71, | | 86, 87, 88, 89, 90, 91, 92, |
| | | 72, 73, 74, 75, 76, 77, 78, | | 93, 94, 95, 96, 97, 98 in |
| | | 79, 80, 81 WO2016014565; | | WO2016014565; Identifier |
| | | Identifier 38 in | | 53 in WO2016187349A1; |
| | | EP3057994Al; Identifier 55 | | Identifier 7 in WO2016094304 A3; |
| | | in WO2016187349A1; | | Identifier 10, 14, 18, 2, 22, |
| | | Identifier 1, 13, 17, 21, 25, | | 26, 30, 34, 38, 42, 46, 50, |
| | | 29, 33, 37, 41, 45, 49, 5, 53, | | 54, 58, 6, 62, 66 in |
| | | 57, 61, 65, 9 in | | WO2016090320; Identifier |
| | | WO2016090320; Identifier | | 100, 102, 175, 96, 98 in |
| | | 101, 743, 174, 758, 95, 759, | | WO2016120216; Identifier |
| | | 97, 760, 99 in WO2016120216; | | 12, 14, 16, 18 in |
| | | Identifier 11, 741, 17 in | | WO2015158671A1; |
| | | WO2015158671Al; | | Identifier 7, 8, 9 in |
| | | Identifier 10, 11, 12, 13, 14 | | WO2016014789; Identifier |
| | | in WO2016014789; | | 14 in WO2016168766A1 |
| | | Identifier 15 in WO2016168766A1 | | |
| humanCD79b | VH | Identifier 27 in WO2016112870 | VL | Identifier 28, 30 in WO2016112870 |
| | | humanCD79b VH 2304 | | |
| | | Identifier 29 in WO2016112870 | | |
| B2MG | VH | Identifier 28 in WO2016126213A1 | VL | Identifier 29 in WO2016126213A1 |
| CD19H 803 | VH | Identifier 28, 29, 32, 33, 34, | — | — |
| | | 35 in WO2016168773 A3; | | |
| | | Identifier 51 in WO2016187349A1; | | |
| | | Identifier 20 in US20160039942; | | |
| | | Identifier 1 in WO2014184143; | | |
| | | Identifier 5 in US20160145337Al; | | |
| | | Identifier 166, 167, 168, 172, | | |
| | | 176, 177, 181, 183, 184, 185, | | |
| | | 62 in US20160152723; | | |
| | | Identifier 15 US20160319020; | | |
| | | Identifier 17, 33, 34, 35 in | | |
| | | EP3057994Al; Identifier 62 | | |
| | | in WO2016097231; | | |
| | | Identifier 12 in WO2016134284; | | |
| | | Identifier 111, 113 in | | |
| | | US20160333114A1 | | |
| NOTCH 2/3 | VH | Identifier 29 in WO2013074596 | VL | Identifier 31 in WO2013074596 |
| KIR (Lirilumab) | VH | Identifier 3 in US20150290316; | VL | Identifier 5 in US20150290316; |
| | | 2371 Identifier 1 in WO2014055648 | | Identifier 2 in WO2014055648 |
| CD22 | VH | Identifier 3 in | VL | Identifier 17, 8, 14, 15 in |
| | | WO2013059593; Identifier | | US20150239974; Identifier |
| | | 10, 11, 12, 7, 9, 8 in | | 7 in US20150299317; |
| | | US20150299317; Identifier | | Identifier 681 in |
| | | 201 in WO2016164731; | | WO2016164731; Identifier |
| | | CD22 VH 869 Identifier | | 682 in WO2016164731; |
| | | 671 in WO2016164731; | | Identifier 683, 2020 in |
| | | Identifier 672 in | | WO2016164731; Identifier |
| | | WO2016164731; Identifier | | 684 in WO2016164731; |
| | | 673 in WO2016164731; | | Identifier 685 in |
| | | Identifier 676 in | | WO2016164731; Identifier |
| | | WO2016164731; Identifier | | 686 in WO2016164731; |
| | | 678 in WO2016164731; | | Identifier 687 in |
| | | Identifier 679 in | | WO2016164731; Identifier |
| | | WO2016164731; Identifier | | 688 in WO2016164731; |
| | | 680 in WO2016164731; | | Identifier 690 in |
| | | Identifier 700 in | | WO2016164731; Identifier |
| | | WO2016164731; Identifier | | 740 in WO2016164731; |
| | | 701 in WO2016164731; | | Identifier 741 in |
| | | Identifier 702 in | | WO2016164731; Identifier |
| | | WO2016164731; Identifier | | 742 in WO2016164731; |
| | | 703 in WO2016164731; | | Identifier 743 in |
| | | Identifier 704 in | | WO2016164731; Identifier |
| | | WO2016164731; Identifier | | 744 in WO2016164731; |
| | | 705 in WO2016164731; | | Identifier 745 in |
| | | Identifier 706 in | | WO2016164731; Identifier |
| | | WO2016164731; Identifier | | 746 in WO2016164731; |
| | | 707 in WO2016164731; | | Identifier 747 in |
| | | Identifier 708 in | | WO2016164731; Identifier |
| | | WO2016164731, Identifier | | 748 in WO2016164731; |
| | | 709 in WO2016164731; | | Identifier 749 in |
| | | Identifier 711 in | | WO2016164731; Identifier |
| | | WO2016164731; Identifier | | 750 in WO2016164731; |
| | | 712 in WO2016164731; | | Identifier 752 in |
| | | Identifier 713 in | | WO2016164731; Identifier |
| | | WO2016164731; Identifier | | 753 in WO2016164731; |
| | | 714 in WO2016164731; | | Identifier 754 in |
| | | Identifier 715 in | | WO2016164731; Identifier |
| | | WO2016164731; Identifier | | 755 in WO2016164731; |
| | | 716 in WO2016164731; | | Identifier 756 in |
| | | Identifier 717 in | | WO2016164731; Identifier |
| | | WO2016164731; Identifier | | 757 in WO2016164731; |
| | | 718 in WO2016164731; | | Identifier 758 in |
| | | Identifier 719 in | | WO2016164731; Identifier |
| | | WO2016164731; Identifier | | 759 in WO2016164731; |
| | | 720 in WO2016164731; | | Identifier 760 in |
| | | Identifier 721 in | | WO2016164731; Identifier |
| | | WO2016164731; Identifier | | 761 in WO2016164731; |
| | | 722 in WO2016164731; | | Identifier 762 in |
| | | Identifier 723 in | | WO2016164731; Identifier |
| | | WO2016164731; Identifier | | 763 in WO2016164731; |
| | | 724 in WO2016164731; | | Identifier 764 in |
| | | Identifier 725 in | | WO2016164731; Identifier |
| | | WO2016164731; Identifier | | 765 in WO2016164731; |
| | | 726 in WO2016164731; | | Identifier 766 in |
| | | Identifier 727 in | | WO2016164731; Identifier |
| | | WO2016164731; Identifier | | 767 in WO2016164731; |
| | | 728 in WO2016164731; | | Identifier 768 in |
| | | Identifier 729 in | | WO2016164731; Identifier |
| | | WO2016164731; Identifier | | 769 in WO2016164731; |
| | | 730 in WO2016164731; | | Identifier 770 in |
| | | Identifier 731 in | | WO2016164731; Identifier |
| | | WO2016164731; Identifier | | 771 in WO2016164731; |
| | | 732 in WO2016164731; | | Identifier 772 in |
| | | Identifier 733 in | | WO2016164731; Identifier |
| | | WO2016164731; Identifier | | 773 in WO2016164731; |
| | | 734 in WO2016164731; | | Identifier 774 in |
| | | Identifier 735 in | | WO2016164731; Identifier |
| | | WO2016164731; Identifier | | 775 in WO2016164731; |
| | | 736 in WO2016164731; | | Identifier 776 in |
| | | Identifier 737 in | | WO2016164731; Identifier |
| | | WO2016164731; Identifier | | 777 in WO2016164731; |
| | | 738 in WO2016164731 | | Identifier 124 in WO2016122701 |
| CTLA4 | VH | Identifier 3, 31, 32, 33, 34, | VL | Identifier 36, 37, 38, 39, 40, 46, 47, |
| | | 35, 41, 42, 43, 44, 45, 7 in | | 48, 49, 50, 8, 4 in US20140105914; |
| | | US20140105914; Identifier 4 | | Identifier 2 in U.S. Pat. No. 8,697,845; |
| | | in U.S. Pat. No. 8,697,845; | | Identifier 20 in US20150283234; |
| | | Identifier 19 in US20150283234; | | Identifier 18 in WO2014066532 |
| | | Identifier 17 in WO2014066532 | | |
| Integrin | VH | Identifier 3, 4, 5 in US | VL | Identifier 10, 11, 8, 9 in US |
| | | 20140161794 | | 20140161794 |
| IL13 | VH | Identifier 302 in US20160168242 | VL | Identifier 303 in US20160168242 |
| Human collagen | VH | Identifier 31 in WO2016112870 | VL | Identifier 32 in WO2016112870 |
| VII | | | | |
| NKG2A | VH | Identifier 32 in WO2016126213A1; | VL | Identifier 33 in WO2016126213A1; |
| | | Identifier 2, 3, 4, 5, 6 in | | Identifier 7 in WO2016041947 |
| | | WO2016041947 | | |
| Campath 1 | VH | Identifier 34 in US20160333114Al | VL | Identifier 31, 33 in US20160333114A1 |
| KIR2DL 1 | VH | Identifier 36 in WO2016126213A1 | VL | Identifier 37 in WO2016126213A1 |
| KIR2DL 2/3 | VH | Identifier 36 in WO2016126213A1 | VL | Identifier 37 in WO2016126213A1 |
| VISTA | VH | Identifier 37, 38, 39, 40 in | VL | Identifier 41, 42, 43, 44, 45 |
| | | WO2015097536 | | in WO2015097536 |
| CD46 | VH | Identifier 39, 47, 59, 15, 19, | VL | Identifier 41, 61, 21, 25, |
| | | 23, 27, 31, 35, 43, 51, 55, | | 29, 33, 37, 45, 49, 53, 57, |
| | | 63, 67, 71, 75, 79, 83, 69, | | 65, 69, 73, 77, 81, 85, 17, |
| | | 71, 83 in WO2012031273; | | 73, 77 in WO2012031273; |
| | | Identifier 1, 10, 11, 12, 13, | | Identifier 23, 24, 25, 26, |
| | | 14, 15, 16, 17, 3, 5, 6, 7, 9, | | 27, 28, 29, 30, 31, 32, 33, |
| | | 18, 19, 20, 21 in WO2016040683 | | 34, 35, 36, 37, 38, 39, 40, |
| | | | | 41, 42 in WO2016040683 |
| RHAMM | VH | Identifier 4 in US20020127227A1 | — | — |
| VEGF | VH | Identifier 4, 8, in | VL | Identifier 2, 6 in |
| | | WO2000034337; Identifier | | WO2000034337; Identifier |
| | | 12, 20, 4, 44 in WO2006012688A1; | | 9 in US20030175276Al; |
| | | Identifier 7 in US20030175276Al; | | Identifier 11, 19, 27, 28, 3, |
| | | Identifier 152, 153, 154, | | 43 in WO2006012688A1; |
| | | 155, 156, 157, 158, 159 in | | Identifier 160, 161, 162, |
| | | US20160090427 | | 163, 164, 165, 166, 167 in |
| | | | | US20160090427 |
| Endoglin | VH | Identifier 41, 42, 43, 71, 73, | VL | Identifier 103, 88, 89, 90, |
| | | 75, 88, 89, 90, 91, 92 in | | 91, 92, 93, 94, 95, 96, 97, |
| | | US20160009811 | | 102, 100 in WO2011041441; |
| | | | | Identifier 100, 102, 103, 3, |
| | | | | 4, 5, 70, 72, 74, 93, 94, 95, |
| | | | | 96, 97 in US20160009811 |
| PRP | VH | Identifier 42 in US20160333114A1 | VL | Identifier 39, 41 in US20160333114Al |
| Lysyloxidase- | VH | Identifier 42, 44 in WO2011097513 | VL | Identifier 43, 45 in WO2011097513 |
| like 2 | | | | |
| PSMA | VH | Identifier 43 in WO2016097231 | VL | Identifier 44 in WO2016097231; |
| | | | | Identifier 44 in WO2016097231 |
| PDL2 | VH | Identifier 43, 44, 56, 46 in | VL | Identifier 47, 48, 49, 50, 51 |
| | | US20130291136 | | in US20130291136 |
| Daclizumab | VH | Identifier 44, 46 in | VL | Identifier 43, 45 in |
| | | US20160333114A1 | | US20160333114Al |
| CD20 | VH | Identifier 45 in WO2016097231; | VL | Identifier 46 in WO2016097231; |
| | | Identifier 11, 13, 14, 15, 16, | | Identifier 10, 12, 8 in WO2017004091; |
| | | 17, 18, 19, 20, 21, 22, 23, 24, | | Identifier 51 in US20160333114Al |
| | | 25, 26, 27, 28, 29, 30, 31, 32, | | |
| | | 33, 7, 9 in WO2017004091; | | |
| | | Identifier 26 in | | |
| | | US20170000900; Identifier | | |
| | | 54 in US20160333114Al; | | |
| | | Identifier 25 in | | |
| | | US20170000900; Identifier | | |
| | | 24 in US20170000900; | | |
| | | Identifier 23 in US20170000900 | | |
| Klon43 | VH | Identifier 47 in WO2016097231 | VL | Identifier 48 in WO2016097231 |
| MUC1 | VH | Identifier 5 in US20160130357; | VL | Identifier 7 in US20160130357; |
| | | Identifier 2, 14 in WO2013023162; | | Identifier 16, 7 in WO2013023162; |
| | | Identifier 15, 19, 23, 60, 64, | | Identifier 17, 21, 25, 62, |
| | | 68 in WO2015116753 | | 66, 70 in WO2015116753 |
| CD4i | VH | Identifier 5 in US20160194375Al | VL | Identifier 4 in US20160194375A1 |
| BAT1 | VH | Identifier 5, 6, 7, 8, 9 in | VL | Identifier 1, 2, 3, 4 in |
| | | WO2013014668 | | WO2013014668 |
| PRAME | VH | Identifier 50, 52, 54, 56, 58, | VL | Identifier 49, 51, 53, 55, |
| | | 60, 62 in WO2016191246A2 | | 57, 59, 61 in WO2016191246A2 |
| CD19 | VH | Identifier 53, 55 in WO2016120216 | VL | Identifier 27, 31 in WO2016168773 |
| | | | | A3; Identifier 49 in WO2016187349A1; |
| | | | | Identifier 11 in WO2016134284; |
| | | | | Identifier 194 in US20140134142Al; |
| | | | | Identifier 54, 56 in WO2016120216; |
| | | | | Identifier 13, 14, 15, 16, 17, |
| | | | | 186, 187, 188, 189, 192, 196, |
| | | | | 197, 198, 199, 200, 201, |
| | | | | 202, 203, 204, 205, 64, 66, |
| | | | | 67, 68, 69, 70, 71, 91 |
| | | | | US20160152723; Identifier |
| | | | | 22 in US20160039942; 3361 |
| | | | | Identifier 63 in WO2016097231; |
| | | | | Identifier 3 in US20160145337Al; |
| | | | | Identifier 112 in US20160333114Al; |
| | | | | Identifier 114 in US20160333114A1; |
| | | | | Identifier 13, 6 US20160319020 |
| Notum | VH | Identifier 56, 331 in WO2012027723 | VL | Identifier 332, 58 in WO2012027723 |
| NOTCH 1 | VH | Identifier 58 in US20160333114A1; | VL | Identifier 16, 20 in WO2013074596; |
| | | Identifier 12 in WO2013074596 | | Identifier 55, 57 in US20160333114Al |
| Osteonectin | VH | Identifier 58 in WO2016112870 | VL | Identifier 59 in WO2016112870 |
| CD74 | VH | Identifier 6 in US20100284906Al; | VL | Identifier 25, 29, 31, 35 in |
| | | Identifier 10, 11, 9 in | | US20130171064; Identifier 12, 13, |
| | | US20040115193Al; | | 14, 11, 4 in US20040115193Al |
| | | Identifier 23, 27, 30, 33 in | | |
| | | US20130171064 | | |
| CD33 | VH | Identifier 65, 67, 69, 71, 77, | VL | Identifier 12, 14, 16, 18 in |
| | | 79, 81, 83, 84 in | | WO2015150526A2; |
| | | WO2016120216; Identifier | | Identifier 66, 68, 70, 72, |
| | | 11, 13, 15; 17 in WO2015150526A2; | | 78, 80, 82 in WO2016120216; |
| | | Identifier 57, 58, 59, 60, 61, | | Identifier 66, 67, 68, 69, 70, |
| | | 62, 63, 64, 65 in WO2016014576 | | 71, 72, 73, 74 in WO2016014576 |
| Rituximab | VH | Identifier 66 in US20160333114Al | VL | Identifier 63, 65 in US20160333114Al |
| GAH | VH | Identifier 7 in US20060057147A1 | VL | Identifier 8 in US20060057147A1 |
| Glyco epitope | VH | Identifier 7 in WO2012007167A1 | VL | Identifier 10 in WO2012007167A1 |
| and ErbBB I | | | | |
| Specific | | | | |
| CD3s | VH | Identifier 7 in WO2014144722A2 | VL | Identifier 8 in WO2014144722A2 |
| IGFR1 | VH | Identifier 7 in WO2015073575 A2 | VL | Identifier 8 in WO2015073575 A2 |
| IL13Ra2 | VH | Identifier 7, 8 in WO2016123143 | — | — |
| N Glycan | VH | Identifier 7, 9 in US20160194375A1 | VL | Identifier 6, 8 in US20160194375A1 N |
| Upar | VH | Identifier 72 in US20160333114Al | VL | Identifier 71, 73 in US20160333114Al |
| CXCR4 | VH | Identifier 72, 73, 74, 75, 84 | VL | Identifier 76, 77, 78, 79, |
| | | in US20110020218 | | 80, 81, 82, 87, 88, 90, 91, |
| | | | | 92, 93 in US20110020218 |
| Tie | VH | Identifier 723 in US20060057138Al | VL | Identifier 724 in US20060057138Al |
| CEA | VH | Identifier 8 in | VL | Identifier 10, 38, 39, 7, 9 in |
| | | U.S. Pat. No. 8,287,865 | | U.S. Pat. No. 8,287,865 |
| Herl/her3 | VH | Identifier 8 of WO2016073629 | VL | Identifier 4 of WO2016073629 |
| CD70 | VH | Identifier 81, 85, 89 in | VL | Identifier 83, 87, 91 in |
| | | WO2015121454 | | WO2015121454; Identifier |
| | | | | 83 in WO2015121454; |
| | | | | Identifier 87 in WO2015121454; |
| | | | | Identifier 91 in WO2015121454 |
| leukocytegenA | VH | Identifier 9 in WO2010065962 A2 | VL | Identifier 24 in WO2010065962 A2 |

In some embodiments, the antigen-binding moiety may comprise an scFv derived from an antibody or antibody fragment that binds to an antigen target such as those described in the cited publications, the contents of each publication are incorporated herein by reference in their entirety for all purposes (Table 2).

TABLE 2
Exemplary antigen-binding moieties comprising an scFv derived from
an antibody or antibody fragment that binds to an antigen target

| Antigen Target | Examples of Source |
|---|---|
| CEA | Identifier 1 in US20160303166A1; Identifier 22 in US20140242701A1 |
| GPC3 | Identifier 1 in WO2016049459; Identifier 12 in US20160208015A1 |
| CS1 | Identifier 1 of WO2016090369; Identifier 17 in WO2014179759A1 |
| VEGFR2 | Identifier 1, 2 in US20120213783 |
| TSLPR | Identifier 1, 2 in US20160311910A1; Identifier 1, 2 in WO2015084513 |
| B7H4 | Identifier 1, 2, 3, 4 in WO2013067492; Identifier 1 in U.S. Pat. No. 9,422,351B2 |
| CD276 | Identifier 10, 19, 28 in US20160053017 |
| GPRC5D | Identifier 100, 101, 102, 103, 104, 105, 106, 107, 108, 109, 110, 111, 112, |
| | 113, 114, 115, 116, 117, 118, 119, 120, 121, 122, 123, 301, 313, 325, 337, |
| | 349, 361, 373, 385 in WO2016090312 |
| WT1/HLA | Identifier 108, 113, 18, 36, 54, 72, 90 in WO2015070061 |
| bispecific | |
| CMet | Identifier 11, 12, 13, 14, 15, 16, 17, 18, 19, 2, 21, 22, 23, 25, 26, 27, 28, 3, |
| | 30, 31, 33, 34, 35, 36, 37, 38, 39, 4; 40, 41, 42, 43, 44, 48, 49, 5, 50, 51, |
| | 52, 53, 54, 55, 56, 57, 58, 6, 60, 7, 9, 29 in US20040166544; Identifier 26, |
| | 27, 28, 29, 30, 32 in US20150299326; Identifier 32 in US20130034559 |
| FcRL | Identifier 11, 15, 19, 23, 27, 31, 35, 39, 3, 43, 7, 594, 596, 598, 600, 602, |
| | 604, 606, 608, 610, 612, 614, 616, 618, 620, 622, 624, 626, 628, 630, 632, |
| | 634, 636, 638, 640, 642, 644, 646, 648, 652, 654, 656; 658, 660, 662, 664, |
| | 666, 668, 670, 672, 674, 676, 680, 682, 684, 686, 688, 690, 692, 694, 696, |
| | 700, 702, 704, 706, 708, 710, 712, 714, 716, 718, 720, 722, 724, 726, 728, |
| | 730, 732, 734, 736, 738, 740, 742, 744, 746, 748, 750, 752, 754, 756, 758, |
| | 760, 762, 764, 766, 768, 770, 772, 774, 776, 778, 780, 782, 784, 786, 788, |
| | 790, 792, 794, 796, 798, 800, 802, 804, 806, 808, 810, 812, 814, 816, 818, |
| | 820, 822, 824, 826, 828, 830, 832, 834, 836, 838, 840, 842, 844; 846, 848, |
| | 850, 852, 854, 856, 858, 860, 862, 864, 866, 868, 870, 874, 876, 878, 880, |
| | 882, 884, 886, 888, 890, 892, 894, 896, 650, 678 in WO2016090337 |
| EGFR | Identifier 11, 38, 41, 44, 47, 50, 53, 56, 59, 62, 65, 68, 71, 74, 77, 80, 83, |
| | 88, 91, 94 in WO2014130657 |
| Claudin | Identifier 11, 5, 7, 9 in WO2016073649A1; Identifier 17 in WO2014179759A1 |
| PSMA diabody | Identifier 12, 13, 14, 15 in WO2011069019 |
| TRBC1 | Identifier 13, 14, 15, 16, 17, 18, 19, 20, 21, 22, 3 in WO2015132598 |
| CD19/CD22 | Identifier 1303, 1307 in WO2016164731A2 |
| Bispecific | |
| CD46 | Identifier 1-42 in WO2016040683 |
| MUC1 | Identifier 15 in US20160130357 |
| MUC3 | Identifier 15 in US20160130357 |
| Folate receptor | Identifier 15 in US20170002072A1 |
| PDK1 | Identifier 15 in WO2016090365 |
| Folate receptor | Identifier 15, 23 in WO2012099973 |
| alpha | |
| BCMA | Identifier 152, 158, 176, 185, 188, 200, 212, 218, 224, 284, 290, 296, 302, |
| | 314, 326, 344, 129, 130, 131, 132, 133, 134, 135, 136, 138, 139, 140, 141, |
| | 142, 143, 144, 145, 146, 147, 148, 149, 263, 264, 265, 266, 271, 273, 39, |
| | 40, 41, 42, 43, 44, 45, 46, 47, 48, 49, 50, 51, 52, 53, 64, 129, 130, 131, |
| | 132, 133, 134, 135, 136, 137, 138, 139, 140, 141, 142, 143, 144, 145, 146, |
| | 147, 148, 149, 263, 264, 265, 266, 39, 40, 41, 42, 43, 44, 45, 46, 47, 48, |
| | 49, 50, 51, 52, 53 in WO2016014565; Identifier 214, 215, 216, 217, 218, |
| | 219, 220, 221, 222, 223, 224, 225, 226, 227, 228, 229, 230, 231, 232, 233, |
| | 234, 235, 236, 237, 238, 239, 240, 241, 242, 243, 244, 245, 246, 247, 248, |
| | 249, 251 in US20160311907A1 |
| CD123 | Identifier 157, 158, 159, 160, 184, 185, 186, 187, 188, 189, 190, 191, 192, |
| | 193, 194, 195, 196, 197, 198, 199, 200, 201, 202, 203, 204, 205, 206, 207, |
| | 208, 209, 210, 211, 212, 213, 214, 215, 478, 480, 483, 485 in WO2016028896; |
| | Identifier 36 in WO2015092024A2; Identifier 57 in WO2016115482A1; |
| | Identifier 36 in EP3083691A2; Identifier 157 in US20160311907A1 |
| CD124 | Identifier 158 in US20160311907A1 |
| CD125 | Identifier 159 in US20160311907A1 |
| CD5 | Identifier 16 in WO2016138491 |
| CD126 | Identifier 160 in US20160311907A1 |
| CD127 | Identifier 161 in US20160311907A1 |
| CD128 | Identifier 162 in US20160311907A1 |
| CD129 | Identifier 163 in US20160311907A1 |
| CD130 | Identifier 164 in US20160311907A1 |
| Claudin6 | Identifier 164 in WO2016115482A1 |
| CD131 | Identifier 165 in US20160311907A1 |
| Claudin7 | Identifier 165 in WO2016115482A1 |
| GCN4 | Identifier 165, 166, 167, 168, 169, 170 in WO2016168773 A3 |
| Claudin8 | Identifier 166 in WO2016115482A1 |
| VEGF | Identifier 168, 169, 170, 171, 172, 173, 174, 175 in US20160090427; |
| | Identifier 498, 500, 502, 504, 506, 508 in US20110177074A1 |
| MUC2 | Identifier 17 in US20160130357 |
| MUC4 | Identifier 17 in US20160130357 |
| CD44 | Identifier 17 in WO2016042461A1 |
| IL4 | Identifier 17, 16 in WO2009121847 |
| ALK | Identifier 17, 18, 19, 20, 21, 22, 23 in WO2015069922; Identifier 17, 18, |
| | 19, 20, 21, 22, 23, 24 in US20160280798A1; Identifier 24 in WO23015069922 |
| ESKAVT | Identifier 173 in WO2016115482A1 |
| GD2 | Identifier 19, 20, 21, in WO2016134284; Identifier 8 in WO2015132604 |
| PSMA | Identifier 19, 21, 30, 31, 34, 35 in WO2012145714 |
| TOSO | Identifier 2 in EP3098237A1 |
| CSPG4 | Identifier 2 in WO2015080981; Identifier 2 in EP3074025A1 |
| CLDN6 | Identifier 2 in WO2016150400 |
| Integrin | Identifier 2, 1 in WO2009070753 |
| bivalent | |
| CD30 | Identifier 20 in WO2016116035A1; Identifier 2 in US20160200824A1 |
| NYBR1 | Identifier 21 in US20160333422A1; Identifier 21, 18, 19 in |
| | WO2015112830 |
| CD37 | Identifier 21, 22 in US20170000900 |
| PDL1 nanobody | Identifier 22, 23, 24, 25, 26, 27 in US20110129458 |
| Radiation | Identifier 22, 24 in WO2005042780A1 |
| inducible | |
| neoantigen | |
| E7MC | Identifier 223, 224, 225, 226, 227, 228, 229, 230, 231, 232 in |
| | WO2016182957A1 |
| TRBC2 | Identifier 23, 24, 25, 26, 27, 28, 29, 30, 31, 32 in WO2015132598 |
| GPC4 | Identifier 24 in WO2016049459 |
| ERBB2 | Identifier 26, 27 in US20110059076A1; Identifier 1, 2 in U.S. Pat. No. 7,244,826 |
| CD33 | Identifier 262, 263, 264, 265, 266, 267, 268, 39, 40, 41, 42, 43, 44, 45, 46, |
| | 47 in WO2016014576; Identifier 37 in WO2015092024 A2; Identifier 37 |
| | in EP3083691A2; Identifier 153, 154, 155, 156, 157, 158, 159, 160, 161, |
| | 162, 163 in WO2016115482A1 |
| PDL2 nanobody | Identifier 28, 29, 30, 31, 32, 33 in US20110129458 |
| 6462 | |
| O-acetylated GD2 | Identifier 29, 31 in US20150140023 |
| ganglioside | |
| OX40 | Identifier 33 in US20150190506 |
| CD79b | Identifier 33 in US20160208021 |
| Human CD79b | Identifier 33 in WO2016112870 |
| CD33/CD3s | Identifier 33, 34, 84 in WO2014144722A2 |
| bispecifc | |
| RORI | Identifier 34 in EP3083691A2; Identifier 249, 250 251, 252, 253, 254, |
| | 255, 256, 257, 258, 259, 260, 261, 262, 263, 264, 265, 266, 267, 268 in |
| | US20160208018A1; Identifier 57 in EP3083671A1; Identifier 1, 2 in |
| | US20160304619A1; Identifier 34 in WO2015092024A2 |
| Human collagen | Identifier 34 in WO2016112870 |
| VII | |
| Trastuzumab | Identifier 35 in US20160208021; Identifier 35 in WO2016112870 |
| Rituximab | Identifier 36 in US20160208021; Identifier 36 in WO2016112870 |
| CD138 | Identifier 36 in WO2016130598A1 |
| Cetuximab | Identifier 37 in WO2016112870; Identifier 37 in US20160208021 |
| Nivolumab | Identifier 38 in US20160208021; Identifier 38 in WO2016112870 |
| Ipilimumab | Identifier 39 in US20160208021; Identifier 39 in WO2016112870 |
| PD1 | Identifier 39, 40, 41, 42, 43, 44, 45, 46, 47, 48, 49, 50, 51, 52, 53, 54, 55, |
| | 56, 57, 58, 59, 60, 61 in US20160311917A1 |
| CLL1 | Identifier 39, 40, 41, 42, 43, 44, 45, 46, 48, 49, 50, 51 in WO2016014535; |
| | Identifier 200, 201, 202, 203, 204, 205, 206, 207, 208, 209, 210, 211, 212, |
| | 213 in US20160311907A1 |
| CLDN7 | Identifier 4 in WO2016150400 |
| Ranibizumab | Identifier 40 in US20160208021; Identifier 40 in WO2016112870 |
| Adalimumab | Identifier 41 in US20160208021; Identifier 41 in WO2016112870 |
| Teplizumab | Identifier 42 in US20160208021 |
| (mutated) | |
| Teplizumab | Identifier 42 in WO2016112870 |
| CD3 | Identifier 46, 47 in WO2015153912A1 |
| EGFR VIII | Identifier 5 in US20140037628; Identifier 174 in US20160311907A1; |
| | Identifier 38 in U.S. Pat. No. 9,394,368B2; Identifier 5 in US20160200819A1 |
| CD22 | Identifier 5, 6 in WO2013059593; Identifier 9 in US20150299317; |
| | Identifier 131, 132, 133, 134, 135, 136, 137, 138, 139, 140, 203, 209, 215, |
| | 221, 227, 232, 238, 244, 250, 256, 262, 268, 274, 280, 286, 292, 298, 304, |
| | 310, 316, 322, 328, 334, 340, 346, 353, 358, 364, 370, 376, 383, 388, 394, |
| | 400, 406, 412, 418, 423 in WO2016164731A2 |
| CD19 | Identifier 53, 54, 37 in EP3083671A1; Identifier 1, 10, 11, 12, 2 in |
| | WO2015157252; Identifier 10, 2, 206, 207, 208, 209, 210, 211, 213, 214, |
| | 215, 216, 217, 218, 219, 220, 221, 222, 223, 224, 225, 4, 45, 47, 49, 51, |
| | 53, 55, 57, 51, 53, 55, 57, 59, 6, 8, 87 in WO2016033570; Identifier 3, 4, |
| | 5, 59, 6, 7, 8, 9 in WO2015157252; 5754 Identifier 5 in |
| | WO2015155341A1; Identifier 7 in WO2014184143; Identifier 9 in |
| | WO2016139487; Identifier 10, 2, 206, 207, 208, 209, 210, 211, 212, 213, |
| | 214, 215, 216, 217, 218, 219, 220, 221, 222, 223, 224, 225, 4, 45, 47, 49, |
| | 51, 53, 55, 57, 59, 6, 8, 87, 89 in US20160152723; Identifier 32, 35, 38 in |
| | EP3083691A2; Identifier 174 in WO2016115482A1; Identifier 20 in |
| | WO2012079000; Identifier 32, 33, 35, 38 in WO2015092024A2; |
| | Identifier 39, 40, 41, 42, 43, 44, 45, 46, 47, 48, 49, 50, 51 in |
| | WO2016109410; Identifier 5, 6 in WO2015155341A1; Identifier 7, 9 in |
| | US20160145337A1; Identifier 20 in U.S. Pat. No. 9,499,629B2; Identifier 73 in |
| | WO2016164580; Identifier 10, 2, 206, 207, 209, 210, 212, 216, 218, 219, |
| | 220, 221, 222, 223, 224, 225, 4, 45, 47, 49, 51, 53, 55, 57, 59, 6, 8, 87, 89 |
| | in US20160152723; Identifier 5 in WO2016055551 |
| END 0180 | Identifier 6 in WO2013098813 |
| CLDN8 | Identifier 6 in WO2016150400 |
| PRAME | Identifier 63, 64, 65, 66, 67, 68, 69 in WO2016191246A2 |
| CD20 | Identifier 691 in WO2016164731A100; Identifier 692 in |
| | WO2016164731A101; Identifier 693 in WO2016164731A102; Identifier |
| | 694 in WO2016164731A103; Identifier 695 in WO2016164731A104; |
| | Identifier 696 in WO2016164731A105; Identifier 175 in WO2016115482A1 |
| Mesothelin | Identifier 7 WO2015188141; Identifier NO 47, 46, 57, 48, 49, 50, 51, 53, |
| | 54, 55, 56, 58, 59, 62, 64, 65, 66, 67, 68, 69, 70, 52, 60, 61, 63 in |
| | WO2016090034; Identifier 10, 11, 12 in WO2013142034; Identifier 11 in |
| | WO2013063419 |
| CCR4 | Identifier 7, 9 in WO2015191997 |
| Mec/CD3s | Identifier 78 in WO2014144722A2 |
| bispecific | |
| Activated alpha v | Identifier 8, 2, 4 in US20090117096A1 |
| beta 3 | |
| RAS | Identifier 81 in WO2016154047 |
| CXCR4 | Identifier 83, 85, 86, 89 in US20110020218 |
| HER2/CD3 | Identifier 9 in WO2014144722 A2 |
| FOLRl/CD3s | Identifier 90 in WO2014144722A2 |
| bispecific | |
| B7H3 | Identifier 99, 100, 101, 102, 103, 104, 102, 17, 18, 19, 20, 21, 22, 23, 24, |
| | 25, 26, 27, 87, 88, 89, 90, 91, 92, 94, 95, 96, 97, 98 in WO2016033225 |

In some embodiments, the antigen-binding moiety may comprise an antigen-binding moiety derived from a CAR that binds to an antigen target, such as those described in the cited publications, the contents of each publication are incorporated herein by reference in their entirety for all purposes (Table 3).

TABLE 3
Exemplary antigen-binding moieties comprising an antigen-binding
moiety derived from a CAR that binds to an antigen target

| Antigen Target | Examples of Source |
|---|---|
| CAR or gate (DC19 | Identifier 1 in US20160296562 |
| or DC33) | |
| CD19 or CD33 | Identifier 1 in WO2015075468 which recognizes CD19 OR CD33 |
| CD19/IL13 bispecific | Identifier 10 in US20160340649A1 |
| CAT19, campana | Identifier 10 in WO2016139487 |
| architecture | |
| CD2 | Identifier 10, 11 in WO2016138491 |
| CD70 | Identifier 100, 93, 94, 96, 101, 95, 97, 98 in WO2015121454 |
| VNAR | Identifier 105, 106, 107, 108, 109, 110 in US20160333094A1 |
| FcRL5 | Identifier 11 in US20170008963A1 |
| CAT19 CAR with | Identifier 11 in WO2016139487 |
| OX40 zeta | |
| endodomain | |
| Folate receptor | Identifier 12 in US20170008963A1 |
| GD2 | Identifier 12 in U.S. Pat. No. 9,446,105B2; Identifier 273, 274 in |
| | WO2016168773A3; Identifier 26, 27, 28, 29, 30, 31, 32, 33, 34, 35, |
| | 36, 37 in WO2015132604; WO2016134284 (no Identifier) |
| CD19 | Identifier 12 in U.S. Pat. No. 9,499,629B2; Identifier 24 in US20160333108A1; |
| | Identifier 25, 29 in US20160333108A1; Identifier 27 in |
| | US20160333108A1; Identifier 1 in EP2997134A4; Identifier 19, 20 |
| | in EP3071687A1; Identifier 181 in WO2016168773A3; Identifier 2 |
| | in WO2015157399A9; Identifier 56, 62 in WO2016174409A1; |
| | Identifier 145, 293, 294, 295, 296, 297, 298 in WO2016179319A1; |
| | Identifier 73 in WO2013176915A1; Identifier 73 in |
| | WO2013176916A1; Identifier 73 in US20130315884A1; Identifier |
| | 73 in US20140134142A1; Identifier 73 in US20150017136A1; |
| | Identifier 73 in US20150203817A1; Identifier 73 in |
| | US20160120905A1; Identifier 73 in US20160120906A1; Identifier |
| | 8, 5 in WO2015124715; Identifier 73 in WO2014184744; Identifier |
| | 73 in WO2014184741; Identifier 14, 15 in US20160145337A1; |
| | Identifier 14, 15 in WO2014184143; Identifier 15, 16 in |
| | WO2015075175; Identifier 16 in US20160145337A1; Identifier 16 in |
| | WO2014184143; Identifier 12 in WO2012079000; Identifier 31, 32, |
| | 33, 34, 35, 36, 37, 38, 39, 40, 41, 42, 58 in WO2016164580; |
| | Identifier 14, 15 in US20160296563A1; Identifier 31, 32, 33, 34, 35, |
| | 36, 37, 38, 39, 40, 41, 42 in WO2015157252; Identifier 14, 15 in |
| | WO2016139487; Identifier 53, 54, 55, 56, 57, 58 in |
| | US20160250258A1; Identifier 1, 2, 3, 4, 5, 6, 7, 8, 9, 10, 11, 12, 13 |
| | in WO2015187528; Identifier 31, 32, 33, 34, 35, 36, 37, 38, 39, 40, |
| | 41, 42, 58 in WO2015157252; Identifier 31, 32, 33, 34, 35, 36, 37, |
| | 38, 39, 40, 41, 42 in WO2014153270; WO2016134284 (no |
| | Identifier); Identifier 13 in WO2016139487 |
| CD3 | Identifier 12 in WO2016138491 |
| CAT19 CAR with | Identifier 12 in WO2016139487 |
| CD28 zeta | |
| endodomain | |
| FRa | Identifier 13, 14 in US20120213783 |
| CD4 | Identifier 13, 14 in WO2016138491 |
| FR beta | Identifier 13, 22 in U.S. Pat. No. 9,402,865B2; Identifier 2, 4, 6 in U.S. Pat. No. 9,446,105B2 |
| CD19/CD20 | Identifier 1308 in WO2016164731A2; Identifier 2, 8, 11 in |
| bispecific | U.S. Pat. No. 9,447,194B2 |
| CLL1 | Identifier 148 in WO2016179319A1; Identifier 35, 36, 37, 38, 39, 40, |
| | 41, 42, 43, 44, 45, 46, 47, 48, 49, 50, 51, 52, 53, 54, 55, 56, 57, 58, |
| | 59, 60, 61, 62, 63, 64, 65, 66, 67, 68, 69, 70, 71, 72, 73, 74, 75, 76, |
| | 77, 78, 79, 80, 81, 82, 83, 84, 85, 86, 87, 88, 89, 90, 91, 92, 93, 94, |
| | 95, 96, 97, 98, 99, 100, 101, 102, 103, 104, 105, 106, 107, 108, 109, |
| | 110, 111, 112 in WO2016120218; Identifier 91, 92, 93, 94, 95, 96, |
| | 97, 98, 99, 100, 101, 102, 103, 197 in WO2016014535 |
| CD5 | Identifier 15, 13 in WO2016138491 |
| EGFR vIII | Identifier 15, 16, 17, 18, 24, 25, 26, 27 in WO2016016341; Identifier |
| | 5, 10, 12, 8, 31, 30, 3 in US20160311907A1; Identifier 10 in |
| | US20160200819A1; Identifier 43, 49, 55, 61, 67, 73, 79, 85, 90, 96 |
| | in U.S. Pat. No. 9,394,368B2; Identifier 49, 55, 61, 67, 73, 79, 85, 90, 2, 1 in |
| | US20170008963A1; Identifier 10, 11 in US20140037628 |
| CD7 | Identifier 17 in WO2016138491 |
| CD52 | Identifier 18 in WO2016138491 |
| BCMA | Identifier 180, 162, 168, 174, 144, 150, 186, 192, 198, 204, 210, 156, |
| | 216, 222, 228, 234, 240, 246, 252, 258, 264, 270, 276 330, 282, 300, |
| | 306, 336, 354, 288, 312, 294, 342, 324, 318, 348 in |
| | WO2016168595A1; Identifier 48, 49, 50, 51, 52, 53, 54, 55, 56, 57, |
| | 58, 59, 19, 20, 21, 22, 23, 24, 25, 26, 27, 28, 29, 30, 31, 32, 33, 34, |
| | 35, 36, 37, 38, 39, 40, 41, 42 in WO2015158671A1; Identifier 124, |
| | 114, 115, 116, 117, 118, 119, 120, 121, 122, 123, 125, 126, 127, 128, |
| | 234, 235, 236, 237, 238, 239, 240, 241, 242, 243, 244, 245, 246, 247, |
| | 248, 249, 250, 251, 252, 253, 254, 267, 268, 269, 270 in |
| | WO2016014565; Identifier 1, 2, 3, 4, 5, 20 in WO2015052538; |
| | Identifier 1, 2, 3, 4, 5, 6 in US20160237139A1; Identifier 9 in |
| | WO2016094304 A3; Identifier 4, 5, 6, 8, 9, 10, 11, 12 in |
| | WO2013154760; Identifier 15, 16, 17, 18, 19, 20, 21, 22, 23, 24, 25, |
| | 26, 27, 28, 29, 71, 73 in WO2016014789; Identifier 125, 126, 127, |
| | 128, 129, 130, 131, 132, 133, 134, 135, 136, 137, 138, 139, 140, 141, |
| | 145, 146, 147, 148, 149, 150 in WO2016120216; Identifier 102, 106, |
| | 107, 108, 109, 110, 111, 112, 129, 130, 131, 132, 133, 134, 135, 136, |
| | 113, 114, 115, 116, 117, 118, 101, 100, 137, 119, 120, 121, 122, 123, |
| | 124, 125, 126, 127, 128, 103, 104, 105, 213 in WO2016097231 |
| Tan | Identifier 19 in US20160311907A1 recognizes CD19 AND CD33 |
| | using a CD45 phosphatase; Identifier 5 in WO2016026742A1 |
| | recognizes CD19 AND CD33 using a CD148 phosphatase; Identifier |
| | 6 in WO2016026742A1 which recognizes CD19 AND NOT CD33 |
| | and is based on an ITIM containing endodomain from LAIR1; |
| | Identifier 3 in WO2015075468 which recognizes CD19 AND NOT |
| | CD33 based on PTPN6 phosphatase; Identifier 2 in WO2015075468 |
| | which recognizes CD19 AND NOT CD33 and recruits a |
| | PTPN6/CD148 fusion protein to an ITIM containing endodomain |
| GD3 | Identifier 19 in WO2016185035A1; Identifier 20, 21, 22, 23, 24, 25, |
| | 26 in WO2016185035A1; WO2016134284 (no Identifier) |
| CAR and gate (CD19 | Identifier 2 in US20160296562 |
| and CD33) CD148 | |
| phosphatase | |
| CD30 | Identifier 20 in WO2016008973A1; Identifier 1 in WO2016116035A1; |
| | WO2016134284 (no Identifier); Identifier 2 in WO2016008973 |
| CD44 | Identifier 21, 22, 23, 24, 25, 26, 27, 28, 31, 32, 33, 34, 35 in |
| | WO2016042461A1 |
| ROR1 | Identifier 216, 217, 215 in WO2016097231; Identifier 79, 80, 81, 82, |
| | 83, 84, 85, 86, 87, 88, 89, 90, 91, 92, 93, 94, 95, 96, 103, 104, 105, |
| | 106, 107, 108, 109, 110, 111, 112, 113, 114, 115, 116, 117, 118, 119, |
| | 120, 127, 128, 129, 130, 131, 132, 133, 134, 1335, 136, 137, 138, 97, |
| | 98, 99, 100, 101, 102, 121, 122, 123, 124, 125, 126 in WO2016016344A1; |
| | Identifier 386, 387, 388, 389, 390, 391, 392, 393, 394 in WO2016187216A1 |
| FR | Identifier 22 in US20170002072A1 |
| CLDN6 | Identifier 22, 23, 24 in WO2016150400 |
| PD1 | Identifier 23 in WO2016014565; Identifier 26 in WO2015142675 |
| Her1/Her3 bispecific | Identifier 23, 24 in US20160215261A1 |
| HER2 | Identifier 25 in US20160215261A1; Identifier 9, 10 of WO2016073629; |
| | Identifier 17, 28, 98, 110 in US20160333114A1; Identifier 271, |
| | 272 in WO2016168773A3; Identifier 5 in WO2016168769A1 |
| CD20 | Identifier 25 in WO2015157399A9; Identifier 177, 181, 182, 183, |
| | 184, 185, 186, 187, 205, 206, 207, 208, 209, 210, 211, 188, 189, 190, |
| | 191, 192, 193, 176, 212, 194, 195, 196, 197, 198, 199, 200, 201, 202, |
| | 203, 178, 179, 180 in WO2016097231 |
| P5A | Identifier 26, 29, 60 in US20160333422A1 |
| GFR alpha | Identifier 27 in WO2016185035A1 |
| GPC3 | Identifier 28, 29 in WO2016185035A1; Identifier 3, 27, 10, 29, 14, |
| | 30, 31, 18, 33 in WO2016049459; Identifier 22 in US20160215261A1 |
| CD22/CD19 | Identifier 29, 30 in WO2016149578; Identifier 1304 in |
| bispecific | WO2016164731A2 |
| CAR and gate (CD19 | Identifier 3 in US20160296562 |
| or CD33) CD45 | |
| phosphatase | |
| EGFR | Identifier 3, 2in WO2014130657; Identifier 36, 37, 38, 39, 35 in |
| | US20140242701 A; Identifier 43, 96, 49, 55, 61, 67, 73, 79, 85, 90, 1 |
| | in WO2014130657 |
| IL13 | Identifier 30, 31, 32 in WO2016120217 |
| Acid/base leucine | Identifier 34, 35 in WO2016124930 |
| zipper | |
| P5AC1 | Identifier 343, 344, 345, 346 in US20160297884A1 |
| P5AC16 | Identifier 347, 396, 348 in US20160297884A1 |
| P6AP | Identifier 349, 350, 351 in US20160297884A1 |
| PC1C12 | Identifier 352, 353, 354 in US20160297884A1 |
| PD1 | Identifier 355, 356, 357 in US20160297884A1; Identifier 119 in |
| | WO2014153270; Identifier 121 in WO2014153270; Identifier 22, 24, |
| | 63, 64, 65, 66, 67, 68, 69, 70, 71, 72, 73, 74, 75, 76, 77, 78, 79, 80, |
| | 81, 82, 83, 84, 85, 86 in US20160311917A1; Identifier 26, 39 in |
| | WO2016172537A1; Identifier 40 in US20160311907A1; Identifier |
| | 121, 119 in WO2015157252; Identifier 24 in WO2016014565; |
| | Identifier 22 in WO2016014565 |
| COM22 | Identifier 358, 359, 360 in US20160297884A1 |
| DDD1/AD1-based zip | Identifier 36 in WO2016124930 |
| PCI | Identifier 361, 362, 363 in US20160297884A1 |
| P6DY | Identifier 364, 365, 366 in US20160297884A1 |
| DDD1/AD1 zip | Identifier 37 in WO2016124930 |
| CD22 | Identifier 380, 204, 260, 266, 272, 278, 284, 290, 296, 302, 308, 341, |
| | 213, 320, 326, 332, 338, 347, 350, 356, 362, 368, 374, 219, 386, 392, |
| | 398, 404, 410, 416, 421, 427, 225, 230, 1109, 236, 242, 248, 254 in |
| | WO2016164731A2; Identifier 15, 16, 17, 18, 19, 20, 32 in |
| | WO2013059593; Identifier 22, 23, 24 in US20150299317 |
| PSMA | Identifier 39 in WO2015142675; Identifier 28, 29 in |
| | US20160311907A1; Identifier 140, 144, 145, 146, 147, 148, 149, |
| | 150, 167, 168, 169, 170, 171, 172, 173, 174, 151, 152, 153, 154, 155, |
| | 156, 139, 138, 175, 157, 158, 159, 160, 161, 162, 163, 164, 165, 166, |
| | 141, 142, 143, 214 in WO2016097231 |
| CD276 | Identifier 39, 40, 41, 42, 43, 44, 45, 46, 47, 122, 123, 124, 125, 126, |
| | 127, 128, 129, 130 in US20160053017 |
| SNAP | Identifier 395 in WO2016187216A1 |
| SSEA4 | Identifier 396, 397 in WO2016187216A1 |
| CEA | Identifier 4 in WO2016008973A1; Identifier 29, 30 in US20140242701A |
| CAR and not gate | Identifier 4, 5 in US20160296562 |
| (CD19 and not CD33) | |
| IL13Ra2specific | Identifier 4, 5, 6 in WO2016089916A1; Identifier 47, 49 in |
| | WO2016123143; Identifier 51, 53, 55 in WO2016123143; Identifier |
| | 1, 31, 32, 33, 34, 35, 36, 37, 38, 39, 40, 41, 42, 43, 44, 45 in |
| | US20160340649A1 |
| TSLPR | Identifier 40, 41, 42 in WO2016034666A1; Identifier 39, 40, 41, 42, |
| | 43, 44, 45, 46 in WO2015084513; Identifier 39, 40, 41, 42, 43 in |
| | US20160311910A1 |
| CAR and gate (CD19 | Identifier 41 in US20160296562 |
| and GD2) | |
| CAR and gate (CD19 | Identifier 43 in US20160296562 |
| and CD5) | |
| ALK | Identifier 43, 44, 45, 46, 47, 48, 49, 50, 51, 52, 53, 54, 55, 56, 57, 58, |
| | 59, 60, 61, 62, 63, 64, 65, 66, 67, 68, 69, 70, 71, 72, 73, 74, 75, 76, |
| | 77, 78, 79, 80, 81, 82, 84, 85, 86, 87, 88, 89, 90 in WO2015069922 |
| VEGFR2 | Identifier 44, 45, 46 in US20160311910A1; Identifier 10, 11, 12 in |
| | US20120213783 |
| CAR and gate (CD19 | Identifier 45 in US20160296562 |
| and EGFR VIII) | |
| KMA | Identifier 46 in US20160340649A1 |
| Mesothelin | Identifier 47, 48 in US20160340649A1; Identifier 48 in |
| | US20160340649A1; Identifier 27 in WO2016172703A2; Identifier |
| | 18, 19, 20, 21, 22, 23 in WO2013142034; Identifier 3 in WO2013067492 |
| CAR and not gate 1 | Identifier 48 in US20160296562 |
| HIV Env | Identifier 48, 49 in WO2016168766A1; Identifier 4 in |
| | EP2997134A4; Identifier 7, 9, 47, 49 in WO2015077789 |
| CD33 | Identifier 48, 49, 50, 51, 52, 53, 54, 55, 83 in WO2016014576; |
| | Identifier 19, 20, 21, 22, 23, 24, 25, 26, 27, 28, 29, 30, 31, 32, 33, 34, |
| | 35, 36, 37, 38, 39, 40, 41, 42, 48, 49, 50, 51, 52, 53, 54, 55, 56, 57, |
| | 58, 59, 60, 61, 62, 63, 64, 65, 66, 67, 68, 69, 70, 71 in WO2015150526A2 |
| CAR and not gate 2 | Identifier 49 in US20160296562 |
| CD435 | Identifier 5 in EP3074419A2 |
| TOSO | Identifier 5, 4 in WO2015075468 |
| MUC1 | Identifier 5, 7 in WO2013063419; Identifier 51 in US20160340406A1; |
| | Identifier 30, 32, 34 in US20160130357; Identifier 295, 298, 301, |
| | 304, 307, 607, 609, 611, 613 in WO2016130726 |
| CAR and not gate 3 | Identifier 50 in US20160296562 |
| CD8 stalk APRIL | Identifier 51 in US20160296562A1 |
| HSP70 | Identifier 51, 53, 5 in WO2015077789; Identifier 21, 22, 23, 24, 25, |
| | 26, 27, 28, 29 in WO2016120217 |
| APRIL-based CAR | Identifier 53 in US20160296562A1; Identifier 52 in |
| | US20160296562A1 |
| CS1 | Identifier 55, 57, 60, 54, 56, 48, 49, 50, 51, 52, 53, 58, 59, 61, 62 in |
| | WO2015121454; Identifier 28 in WO2014179759A1 |
| CAR and not gate | Identifier 6 in US20160296562A1 |
| (CD19 and not CD33) | |
| Trophoblast | Identifier 6 in WO2015075468; Identifier 4 in US20160347854A1; |
| Glycoprotein 5T4 | Identifier 4 in EP3098237A1; Identifier 19, 20, 21, 22, 23, 24, 25, 26, |
| | 27, 28, 29, 30, 31, 32, 33, 34, 35, 36, 37, 38, 39 in WO2016034666A1 |
| HERVK | Identifier 6 in WO2016168769A1 |
| NCAR with RQR82 | Identifier 615 in WO2016130726 |
| ACD 19 | |
| NYBR1 | Identifier 617, 619 in WO2016130726; Identifier 218 in WO2016097231; |
| | Identifier 26, 29, 60 in WO2015112830; Identifier 1 in US20160333422A1 |
| CD123 | Identifier 69 in WO2016142532; Identifier 23, 24, 25, 26, 27, 28, 29, |
| | 30, 31, 32, 33, 34, 35, 36, 37, 38, 39, 40, 41, 42, 44, 45, 46, 47, 48 in |
| | WO2015140268A1; Identifier 9, 10, 11, 12 in US20140271582; |
| | Identifier 56, 57, 58, 59, 60, 61 in WO2016097231; Identifier 98, 99, |
| | 100, 101, 125, 126, 127, 128, 129, 130, 131, 132, 133, 134, 135, 136, |
| | 137, 138, 139, 140, 141, 142, 143, 144, 145, 146, 147, 148, 149, 150, |
| | 151, 152, 153, 154, 155, 156 in WO2016028896; Identifier 31, 32, |
| | 33, 34, 35, 36, 37, 38, 39, 40, 41, 42, 43, 44, 45, 46, 47, 48, 49, 50, |
| | 51, 52, 53, 54, 55, 56, 57, 58, 59, 60, 61, 62, 63, 64, 65, 66, 67, 68, |
| | 69, 70, 71, 72, 73, 74, 75, 76, 77, 78, 79, 80, 81, 82, 83, 84, 85, 86, |
| | 87, 88, 89, 90, 91, 92, 93, 94, 95, 96, 97, 98, 99, 100, 101, 102, 103, |
| | 104, 105, 106, 107, 108, 109, 110, 111, 112, 113, 114, 115, 116, 117, |
| | 118, 119, 120, 121, 122, 123, 124, 125, 126, 127, 128, 129, 130, 131, |
| | 132, 133, 134, 135, 136, 137, 138, 139, 140, 141, 142, 143, 144, 145, |
| | 146, 147, 148, 149, 150, 151, 152, 153, 154, 155, 156, 157, 158, 159, |
| | 160, 172, 173, 174, 175, 176, 177, 178, 179, 180, 181, 182, 183, 184, |
| | 185, 186, 187, 188, 189, 190, 191, 193, 194, 195, 196, 197 in |
| | WO2016120220; Identifier 1, 2, 3, 4, 5, 6, 7, 8, 9, 10, 142 in |
| | WO2016120216 |
| CD410 | Identifier 7 in EP3074419A2 |
| CD38 | Identifier 70, 71, 72, 64, 65, 66, 67, 68, 69 in WO2016097231; |
| | Identifier 35, 36, 37 in WO2015121454 |
| GCN4 | Identifier 8, 10 in U.S. Pat. No. 9,446,105B2 |
| CD4-DDY3 | Identifier 9 in EP3074419A2 |
| Fra | Identifier 959 in WO2016090337; Identifier 13 in US20170002072A1 |
| CD70 | Identifier 99 in WO2015121454 |

In some embodiments, the extracellular antigen-binding domain further comprises a leader sequence. The leader sequence may be located at the amino-terminus of the extracellular antigen-binding domain. The leader sequence may be optionally cleaved from the antigen-binding moiety during cellular processing and localization of the CAR to the cellular membrane.

In some embodiments, the leader sequence comprises the amino acid sequence of SEQ ID NO: 15, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% o, sequence identity with SEQ TD NO: 15. In some embodiments, the nucleotide sequence encoding the leader comprises the nucleotide sequence that encodes the amino acid sequence of SEQ TD NO: 15, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% o, sequence identity with SEQ ID NO: 15. In some embodiments, the nucleotide sequence encoding the leader sequence comprises the sequence set forth in SEQ ID NO: 16, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99% o, sequence identity with SEQ ID NO: 16. In some embodiments, the leader sequence comprises the amino acid sequence of SEQ ID NO: 15. In some embodiments, the nucleotide sequence encoding the leader sequence comprises the nucleotide sequence set forth in SEQ ID NO: 16. In some embodiments, the nucleotide sequence encoding the leader sequence comprises the sequence set forth in SEQ ID NO: 37, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 37. In some embodiments, the nucleotide sequence encoding the leader sequence comprises the nucleotide sequence set forth in SEQ ID NO: 37.

### Transmembrane Domain of the CAR

In some embodiments, the CARs expressed by the modified immune effector cell comprise a transmembrane domain. The transmembrane domain may be fused in frame between the extracellular target-binding domain and the cytoplasmic domain.

The transmembrane domain may be derived from the protein contributing to the extracellular target-binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid-binding of proteins naturally associated with the transmembrane domain. In some embodiments, the transmembrane domain includes additional amino acids to allow for flexibility and/or optimal distance between the domains connected to the transmembrane domain.

The transmembrane domain may be derived either from a natural or from a synthetic source. Where the source is natural, the domain may be derived from any membrane-bound or transmembrane protein. Non-limiting examples of transmembrane domains of particular use in this invention may be derived from (i.e. comprise at least the transmembrane region(s) of) the a, R or (chain of the T-cell receptor, CD28, CD3 epsilon, CD45, CD4, CD5, CD8, CD9, CD16, CD22, CD33, CD3γ, CD40, CD64, CD80, CD86, CD134, CD137, or CD154. Alternatively, the transmembrane domain may be synthetic, in which case it will comprise predominantly hydrophobic residues such as leucine and valine. For example, a triplet of phenylalanine, tryptophan and/or valine can be found at each end of a synthetic transmembrane domain.

In some embodiments, the transmembrane domain may be derived from CD8a, CD28, CD8, CD4, CD3ζ, CD40, CD134 (OX-40), NKG2A/C/D/E, or CD7. In some embodiments, the transmembrane domain may be derived from CD28.

In some embodiments, it will be desirable to utilize the transmembrane domain of the ζ, η, or FcεR1γ chains which contain a cysteine residue capable of disulfide bonding, so that the resulting chimeric protein will be able to form disulfide linked dimers with itself, or with unmodified versions of the ζ, η or FcεR1γ chains or related proteins. In some instances, the transmembrane domain will be selected or modified by amino acid substitution to avoid-binding of such domains to the transmembrane domains of the same or different surface membrane proteins to minimize interactions with other members of the receptor complex. In other cases, it will be desirable to employ the transmembrane domain of ζ, η or FcεR1γ and −β, MB1 (Igα.), B29 or CD3-γ, ζ, or η, in order to retain physical association with other members of the receptor complex.

In some embodiments, the transmembrane domain is derived from CD3ζ, CD28, CD4, or CD8a. In some embodiments, the transmembrane domain is derived from CD3ζ and optionally comprises the amino acid sequence SEQ ID NO: 23, or specified portion or variant thereof. In some embodiments, the transmembrane domain is derived from CD28 and optionally comprises the amino acid sequence SEQ ID NO: 31, or specified portion or variant thereof. In some embodiments, the transmembrane domain is derived from CD8α and optionally comprises the amino acid sequence SEQ ID NO: 49, or specified portion or variant thereof. In some embodiments, the transmembrane domain is derived from CD4 and optionally comprises the amino acid sequence SEQ ID NO: 51, or specified portion or variant thereof.

In a specific embodiment, the transmembrane domain is derived from the CD3ζ transmembrane domain. In some embodiments, the CD3ζ transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 23. In some embodiments, the nucleotide sequence that encodes the CD3ζ transmembrane domain comprises the nucleotide sequence that encodes the amino acid sequence of SEQ ID NO: 23, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 23. In some embodiments, the nucleotide sequence that encodes the CD3ζ transmembrane domain comprises the nucleotide sequence set forth in SEQ ID NO: 24, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 24. In some embodiments, the CD3ζ transmembrane domain comprises the amino acid sequence set forth in SEQ ID NO: 23. In some embodiments, the nucleotide sequence that encodes the CD3ζ transmembrane domain comprises the nucleotide sequence set forth in SEQ ID NO: 24.

In a specific embodiment, the transmembrane domain is derived from the CD28 transmembrane domain. In some embodiments, the CD28 transmembrane domain comprises the amino acid sequence set forth in SEQ ID NO: 31, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 31. In some embodiments, the nucleotide sequence that encodes the CD28 transmembrane domain comprises the nucleotide sequence that encodes the amino acid sequence of SEQ ID NO: 31, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 31. In some embodiments, the nucleotide sequence that encodes the CD28 transmembrane domain comprises the nucleotide sequence set forth in SEQ ID: 32, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 32. In some embodiments, the CD28 transmembrane domain comprises the amino acid sequence set forth in SEQ ID NO: 31. In some embodiments, the nucleotide sequence that encodes the CD28 transmembrane domain comprises the nucleotide sequence set forth in SEQ ID NO: 32.

In a specific embodiment, the transmembrane domain is derived from the CD8α transmembrane domain. In some embodiments, the CD8α transmembrane domain comprises the amino acid sequence set forth in SEQ ID NO: 49, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 49. In some embodiments, the nucleotide sequence that encodes the CD8α transmembrane domain comprises the nucleotide sequence that encodes the amino acid sequence of SEQ ID NO: 49, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 49. In some embodiments, the nucleotide sequence that encodes the CD8α transmembrane domain comprises the nucleotide sequence set forth in SEQ ID NO: 50, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 50. In some embodiments, the CD8α transmembrane domain comprises the amino acid sequence set forth in SEQ ID NO: 49. In some embodiments, the nucleotide sequence that encodes the CD8α transmembrane domain comprises the nucleotide sequence set forth in SEQ ID NO: 50.

In a specific embodiment, the transmembrane domain is derived from the CD4 transmembrane domain. In some embodiments, the CD4 transmembrane domain comprises the amino acid sequence set forth in SEQ ID NO: 51, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 51. In some embodiments, the nucleotide sequence that encodes the CD4 transmembrane domain comprises the nucleotide sequence that encodes the amino acid sequence of SEQ ID NO: 51, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 51. In some embodiments, the nucleotide sequence that encodes the CD4 transmembrane domain comprises the nucleotide sequence set forth in SEQ ID NO: 52, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 52. In some embodiments, the CD4 transmembrane domain comprises the amino acid sequence set forth in SEQ ID NO: 51. In some embodiments, the nucleotide sequence that encodes the CD4 transmembrane domain comprises the nucleotide sequence set forth in SEQ ID NO: 52.

In some embodiments, the CAR further comprises a linker domain between the extracellular antigen-binding domain and the transmembrane domain, wherein the antigen-binding domain, linker, and the transmembrane domain are in frame with each other.

The term “linker domain” as used herein generally means any oligo- or polypeptide that functions to link the antigen-binding moiety to the transmembrane domain. A linker domain can be used to provide more flexibility and accessibility for the antigen-binding moiety. A linker domain may comprise up to 300 amino acids, preferably 10 to 100 amino acids and most preferably 25 to 50 amino acids. A linker domain may be derived from all or part of naturally occurring molecules, such as from all or part of the extracellular region of CD8, CD4 or CD28, or from all or part of an antibody constant region. Alternatively, the linker domain may be a synthetic sequence that corresponds to a naturally occurring linker domain sequence, or may be an entirely synthetic linker domain sequence. Non-limiting examples of linker domains which may be used in accordance to the invention include a part of human CD8α chain, partial extracellular domain of CD28, FcγRIIIa receptor, IgG, IgM, IgA, IgD, IgE, an Ig hinge, or functional fragment thereof. In some embodiments, additional linking amino acids are added to the linker domain to ensure that the antigen-binding moiety is an optimal distance from the transmembrane domain. In some embodiments, when the linker is derived from an Ig, the linker may be mutated to prevent Fc receptor binding.

In some embodiments, the linker domain comprises a hinge region. Non-limiting examples of hinge regions suitable for use in the present invention may be derived from an immunoglobulin IgG hinge or functional fragment, including IgG1, IgG2, IgG3, IgG4, IgM1, IgM2, IgA1, IgA2, IgD, IgE, or a chimera or variant thereof. In some embodiments, the hinge region comprises an amino acid sequences SEQ ID NO: 19, or specified portion or variant thereof. In some embodiments, the hinge region comprises an amino acid sequences SEQ ID NO: 40, or specified portion or variant thereof. In some embodiments, the linker region comprises an amino acid sequences SEQ ID NO: 21, or specified portion or variant thereof.

In some embodiments, the hinge region comprises the amino acid sequence SEQ ID NO: 19, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 19. In some embodiments, the nucleotide sequence encoding the hinge region comprises the nucleotide sequence that encodes the amino acid sequence of SEQ ID NO: 19, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 19. In some embodiments, the nucleotide sequence encoding the hinge region comprises the sequence set forth in SEQ ID NO: 20, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 20. In some embodiments, the hinge region comprises the amino acid sequence of SEQ ID NO: 19. In some embodiments, the nucleotide sequence encoding the hinge region comprises the nucleotide sequence set forth in SEQ ID NO: 20.

In some embodiments, the linker domain comprises a hinge region which is an IgG1 hinge. In some embodiments, the IgG1 hinge comprises the amino acid sequence SEQ ID NO: 40, or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 40. In some embodiments, the nucleotide sequence encoding the IgG1 hinge comprises the nucleotide sequence that encodes the amino acid sequence of SEQ ID NO: 40, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 40. In some embodiments, the nucleotide sequence encoding the IgG1 hinge comprises the sequence set forth in SEQ ID NO: 41, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 41. In some embodiments, the hinge region comprises the amino acid sequence of SEQ ID NO: 40. In some embodiments, the nucleotide sequence encoding the IgG1 hinge comprises the nucleotide sequence set forth in SEQ ID NO: 41.

In some embodiments, the linker domain comprises the amino acid sequence SEQ ID NO: 21. or a or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 21. In some embodiments, the nucleotide sequence encoding the linker domain comprises the nucleotide sequence that encodes the amino acid sequence of SEQ ID NO: 21, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 21. In some embodiments, the nucleotide sequence encoding the linker domain comprises the sequence set forth in SEQ ID NO: 22, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 22. In some embodiments, the linker domain comprises the amino acid sequence of SEQ ID NO: 21. In some embodiments, the nucleotide sequence encoding the linker domain comprises the nucleotide sequence set forth in SEQ ID NO: 22. In some embodiments, the nucleotide sequence encoding the linker domain comprises the sequence set forth in SEQ ID NO: 42, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 42. In some embodiments, the nucleotide sequence encoding the linker domain comprises the nucleotide sequence set forth in SEQ ID NO: 42.

### Cytoplasmic Domain of the CAR

In some embodiments, the CAR expressed by the immune effector cell described herein further comprises a cytoplasmic domain. In some embodiments, the cytoplasmic domain of the CAR comprises one or more lymphocyte activation domains.

The cytoplasmic domain, which comprises the lymphocyte activation domain of the CAR, is responsible for activation of at least one of the normal effector functions of the lymphocyte in which the CAR has been placed in. The term “effector function” refers to a specialized function of a cell. Effector function of a T cell, for example, may be cytolytic activity or helper activity including the secretion of cytokines. Thus, the term “lymphocyte activation domain” refers to the portion of a protein which transduces the effector function signal and directs the cell to perform a specialized function. While usually the entire lymphocyte activation domain is present, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the lymphocyte activation domain sufficient to transduce the effector function signal.

Non-limiting examples of lymphocyte activation domains which can be used in the CARs described herein include those derived from DAP10, DAP12, Fc epsilon receptor I γ chain (FCER1G), CD3δ, CD3ε, CD3γ, CD3ζ, CD27, CD28, CD40, CD134, CD137, CD226, CD79A, ICOS, and MyD88. In some embodiments, the lymphocyte activation domain used in the CARs described herein may be derived from CD3ζ and optionally comprise the amino acid sequence SEQ ID NO: 25, or specified portions or variants thereof.

In some embodiments, the lymphocyte activation domain is derived from CD3ζ and comprises the amino acid sequence SEQ ID NO: 25. In some embodiments, the CD3ζ signaling domain comprises the amino acid sequence set forth in SEQ ID NO: 25 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 25. In some embodiments, the nucleotide sequence that encodes the CD3ζ signaling domain comprises the nucleotide sequence that encodes the amino acid sequence of SEQ ID NO: 25, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 25. In some embodiments, the nucleotide sequence that encodes the CD3ζ signaling domain comprises the nucleotide sequence set forth in SEQ ID NO: 26, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 26. In some embodiments, the CD3ζ signaling domain comprises the amino acid sequence set forth in SEQ ID NO: 25. In some embodiments, the nucleotide sequence that encodes the CD3ζ signaling domain comprises the nucleotide sequence set forth in SEQ ID NO: 26. In some embodiments, the nucleotide sequence that encodes the CD3ζ signaling domain comprises the nucleotide sequence set forth in SEQ ID NO: 44, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 44. In some embodiments, the nucleotide sequence that encodes the CD3ζ signaling domain comprises the nucleotide sequence set forth in SEQ ID NO: 44.

In some embodiments, the cytoplasmic domain further includes one or more co-stimulatory domains. Non-limiting examples of co-stimulatory domains that may be used in the CARs described herein include those derived from 4-1BB (CD137), CD28, ICOS, CD134 (OX-40), BTLA, CD27, CD30, GITR, CD226, HVEM, MyD88, IL-2Rβ, or the STAT3-binding YXXQ. In some embodiments, the CAR of the present disclosure comprises one co-stimulatory domain. In some embodiments, the CAR of the present disclosure comprises a co-stimulatory domain derived from CD28.

In some embodiments, the co-stimulatory domains which can be used in the CARs of the present disclosure may be derived from CD28, 4-1BB, CD27, CD40, CD134, CD226, CD79A, ICOS, or MyD88, or any combination thereof.

In some embodiments, the CAR of the present disclosure comprises one or more co-stimulatory domains. In some embodiments, the CAR of the present disclosure comprises two or more co-stimulatory domains. In certain embodiments, the CAR of the present disclosure comprises two, three, four, five, six or more co-stimulatory domains. For example, the CAR of the present disclosure may comprise a co-stimulatory domain derived from 4-1BB and a co-stimulatory domain derived from CD28.

In certain embodiments, the CARs of the present disclosure comprise a cytoplasmic domain, which comprises a signaling domain, a MyD88 polypeptide or functional fragment thereof, and a CD40 cytoplasmic polypeptide region or a functional fragment thereof. In certain embodiments, the CAR lacks the CD40 transmembrane and/or CD40 extracellular domains. In certain embodiments, the CAR includes the CD40 transmembrane domain. In certain embodiments, the CAR includes the CD40 transmembrane domain and a portion of the CD40 extracellular domain, wherein the CD40 extracellular domain does not interact with natural or synthetic ligands of CD40.

In certain embodiments, the signaling domain is separated from the MyD88 polypeptide or functional fragment thereof and/or the CD40 cytoplasmic polypeptide region or a functional fragment thereof. In certain embodiments, the lymphocyte activation domain is separated from the MyD88 polypeptide or functional fragment thereof and/or the CD40 cytoplasmic polypeptide region or a functional fragment thereof by at least 1, 2, 3, 4, 5, 6, 7, 8, 9, 10, 11, 12, 13, 14, or 15 amino acids.

In some embodiments, the signaling domain(s) and co-stimulatory domain(s) can be in any order. In some embodiments, the signaling domain is upstream of the co-stimulatory domains. In some embodiments, the signaling domain is downstream from the co-stimulatory domains. In the cases where two or more co-stimulatory domains are included, the order of the co-stimulatory domains could be switched.

In some embodiments, the co-stimulatory domain is derived from CD28. In some embodiments, the co-stimulatory domain is derived from CD28 and optionally comprises the amino acid sequence SEQ ID NO: 33, or portion or fragment thereof.

In some embodiments, the co-stimulatory domain is derived from CD28 and comprises the amino acid sequence SEQ ID NO: 33. In some embodiments, the CD28 co-stimulatory domain comprises the amino acid sequence set forth in SEQ ID NO: 33 or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 33. In some embodiments, the nucleotide sequence that encodes the CD28 co-stimulatory domain comprises the nucleotide sequence that encodes the amino acid sequence of SEQ ID NO: 33, or a variant thereof having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 33. In some embodiments, the nucleotide sequence that encodes the CD28 co-stimulatory domain comprises the nucleotide sequence set forth in SEQ ID NO: 34, or a nucleotide sequence having at least 50, at least 55, at least 60, at least 65, at least 70, at least 75, at least 80, at least 85, at least 90, at least 91, at least 92, at least 93, at least 94, at least 95, at least 96, at least 97, at least 98 or at least 99%, sequence identity with SEQ ID NO: 34. In some embodiments, the CD28 co-stimulatory domain comprises the amino acid sequence set forth in SEQ ID NO: 34. In some embodiments, the nucleotide sequence that encodes the CD28 co-stimulatory domain comprises the nucleotide sequence set forth in SEQ ID NO: 34.

In some embodiments, the cytoplasmic domain comprises both the CD3ζ lymphocyte activation domain and the CD28 co-stimulatory domain, which are fused in frame. The CD3ζ lymphocyte activation domain and the CD28 co-stimulatory domain can be in any order. In some embodiments, the CD3ζ lymphocyte activation domain is downstream of the CD28 co-stimulatory domain.

### Accessory Genes of the CAR

In addition to the CAR construct, the CAR may further comprise an accessory gene that encodes an accessory peptide. Examples of accessory genes can include a transduced host cell selection marker, an in vivo tracking marker, a cytokine, a suicide gene, or some other functional gene. In a specific embodiment, the CAR is co-expressed with a truncated CD19 molecule (tCD19). For example, expression of tCD19 can help determine transduction efficiency. In some embodiments, the CAR comprises the tCD19 construct. In some embodiments, the CAR does not include the tCD19 construct. In some embodiments, the tCD19 can be replaced with a functional accessory gene to enhance the effector function of the CAR containing immune effector cells. In some embodiments, the functional accessory gene can increase the safety of the CAR. In some embodiments, the CAR comprises at least one accessory gene. In some embodiments, the CAR comprises one accessory gene. In other embodiments, the CAR comprises two accessory genes. In yet another embodiment, the CAR comprises three accessory genes.

Other examples of additional genes include genes that encode polypeptides with a biological function; examples include, but are not limited to, cytokines, chimeric cytokine receptors, dominant negative receptors, safety switches (CD20, truncated EGFR or HER2, inducible caspase 9 molecules). As another example, the CAR construct may comprise an additional gene which is a synNotch receptor. Once activated, the synNotch receptor can induce the expression of a target gene (e.g., a second CAR and/or bispecific molecule).

In some embodiments, the CAR may comprise one or more additional nucleotide sequences encoding one or more additional polypeptide sequences. As a non-limiting example, the one or more additional polypeptide sequences may be selected from one or more cellular markers, epitope tags, cytokines, safety switches, dimerization moieties, or degradation moieties.

In certain embodiments, the CAR comprises at least one additional gene (i.e., a second gene). In certain embodiments, the CAR comprises one second gene. In other embodiments, the CAR comprises two additional genes (i.e., a third gene). In yet another embodiment, the CAR comprises three additional genes (i.e., a fourth gene). In certain embodiments, the additional genes are separated from each other and the CAR construct. For example, they may be separated by 2A sequences and/or an internal ribosomal entry sites (IRES). In certain examples, the CAR can be at any position of the polynucleotide chain (for example construct A: CAR, second gene, third gene, fourth gene; construct B: second gene, CAR, third gene, fourth gene; etc.)

Non-limiting examples of classes of accessory genes that can be used to increase the effector function of CAR containing immune effector cells, include i) secretable cytokines (e.g., but not limited to, IL-7, IL-12, IL-15, IL-18), ii) membrane bound cytokines (e.g., but not limited to, IL-15), iii) chimeric cytokine receptors (e.g., but not limited to, IL-2/IL-7, IL-4/IL-7), iv) constitutive active cytokine receptors (e.g., but not limited to, C7R), v) dominant negative receptors (DNR; e.g., but not limited to TGFRII DNR), vi) ligands of co-stimulatory molecules (e.g., but not limited to, CD80, 4-1BBL), vii) antibodies, including fragments thereof and bispecific antibodies (e.g., but not limited to, bispecific T-cell engagers (BiTEs)), or vii) a second CAR.

tCD19 may be separated from the CAR-encoding sequence by a separation sequence (e.g., a 2A sequence). tCD19 could also be replaced with two accessory genes separated by a separation sequence (e.g., a 2A sequence) using a combination of the classes of molecules listed above (e.g., CAR-2A-CD20-2A-IL15). In addition, the use of two separation sequences (e.g., 2A sequences) would allow the expression of TCR (e.g., CAR-2A-TCRα-2A-TCRβ). In the constructs with a CAR and two or three accessory genes, the order of the CAR and the 2nd or 3rd transgene could be switched.

In certain embodiments, the additional gene may be regulated by an NFAT dependent-promoter. Activation of the T-cell or other lymphocyte leads to activation of the transcription factor NFAT resulting in the induction of the expression of the protein encoded by the gene linked with the NFAT dependent promoter. One or more members of the NFAT family (i.e., NFATc1, NFATc2, NFATc3, NFATc4, and NFAT5) is expressed in most cells of the immune system. NFAT-dependent promoters and enhancers tend to have three to five NFAT binding sites.

In certain embodiments, the functional additional gene can be a suicide gene. A suicide gene is a recombinant gene that will cause the host cell that the gene is expressed in to undergo programmed cell death or antibody mediated clearance at a desired time. Suicide genes can function to increase the safety of the CAR. In another embodiment, the additional gene is an inducible suicide gene. Non-limiting examples of suicide genes include i) molecules that are expressed on the cell surface and can be targeted with a clinical grade monoclonal antibody including CD20, EGFR or a fragment thereof, HER2 or a fragment thereof, and ii) inducible suicide genes (e.g., but not limited to inducible caspase 9 (see Straathof et al. (2005) Blood. 105(11): 4247-4254; US Publ. No. 2011/0286980, each of which are incorporated herein by reference in their entirety for all purposes)).

In certain aspects, CARs of the present disclosure may be regulated by a safety switch. As used herein, the term “safety switch” refers to any mechanism that is capable of removing or inhibiting the effect of a CAR from a system (e.g., a culture or a subject). Safety switches can function to increase the safety of the CAR.

The function of the safety switch may be inducible. Non-limiting examples of safety switches include (a) molecules that are expressed on the cell surface and can be targeted with a clinical grade monoclonal antibody including CD20, EGFR or a fragment thereof, HER2 or a fragment thereof, and (b) inducible suicide genes (e.g., but not limited to herpes simplex virus thymidine kinase (HSV-TK) and inducible caspase 9 (see Straathof et al. (2005) Blood. 105(11): 4247-4254; US Publ. No. 2011/0286980, each of which are incorporated herein by reference in their entirety for all purposes).

In some embodiments, the safety switch is a CD20 polypeptide. Expression of human CD20 on the cell surface presents an attractive strategy for a safety switch. The inventors and others have shown that cells that express CD20 can be rapidly eliminated with the FDA approved monoclonal antibody rituximab through complement-mediated cytotoxicity and antibody-dependent cell-mediated cytotoxicity (see e.g., Griffioen, M., et al. Haematologica 94, 1316-1320 (2009), which is incorporated herein by reference in its entirety for all purposes). Rituximab is an anti-CD20 monoclonal antibody that has been FDA approved for Chronic Lymphocytic Leukemia (CLL) and Non-Hodgkin's Lymphoma (NHL), among others (Storz, U. MAbs 6, 820-837 (2014), which is incorporated herein by reference in its entirety for all purposes). The CD20 safety switch is non-immunogenic and can function as a reporter/selection marker in addition to a safety switch (Bonifant, C. L., et al. Mol Ther 24, 1615-1626 (2016); van Loenen, M. M., et al. Gene Ther 20, 861-867 (2013); each of which is incorporated herein by reference in its entirety for all purposes).

In some embodiments, the polynucleotide sequence(s) encoding the CARs of the present disclosure may be expressed in an inducible fashion, for example, as may be achieved with an inducible promoter, an inducible expression system, an artificial signaling circuits, and/or drug-induced splicing.

In some embodiments, the polynucleotide sequence(s) encoding the CARs of the present disclosure may be expressed in an inducible fashion, such as that which may be achieved with i) an inducible promoter, for example, but not limited to promotors that may be activated by T cell activation (e.g. NFAT, Nur66, IFNg) or hypoxia; ii) an inducible expression system, for example, but not limited to doxycycline- or tamoxifen-inducible expression system; iii) artificial signaling circuits including, but not limited to, SynNotch, and/or iv) drug-induced splicing.

In some embodiments, the polynucleotide sequence(s) encoding the CARs disclosed herein may be expressed as a “split molecule” in which for example, transmembrane and intracellular signaling regions, or any other domains or regions of the CAR, may be assembled only in the presence of a heterodimerizing small molecule (e.g., small organic molecule, nucleic acid, polypeptide, or a fragment, isoform, variant, analog, or derivative thereof).

In some embodiments, the polynucleotide sequence(s) encoding the CARs herein may further encode a moiety so that the stability of CAR may be regulated with a small molecule, including but not limited to, the “SWIFF” technology or an immunomodulatory drug (IMiD)-inducible degron.

A “separation sequence” refers to a peptide sequence that causes a ribosome to release the growing polypeptide chain that it is being synthesizes without dissociation from the mRNA. In this respect, the ribosome continues translating and therefore produces a second polypeptide. Non-limiting examples of separation sequences includes T2A (EGRGSLLTCGDVEENPGP (SEQ ID NO: 45) or GSGEGRGSLLTCGDVEENPGP (SEQ ID NO: 53)); the foot and mouth disease virus (FMDV) 2A sequence (GSGSRVTELLYRMKRAETYCPRPLLAIIIPTEARHKQKIVAPVKQLLNFDLLKLAGDVES NPGP (SEQ ID NO: 54)); Sponge (Amphimedon queenslandica) 2A sequence (LLCFLLLLLSGDVELNPGP (SEQ ID NO: 55); or HHFMFLLLLLAGDIELNPGP (SEQ ID NO: 56)); acorn worm (Saccoglossus kowalevskii) 2A sequence (WFLVLLSFILSGDIEVNPGP (SEQ ID NO: 57)); amphioxus (Branchiostoma floridae) 2A sequence (KNCAMYMLLLSGDVETNPGP (SEQ ID NO: 58); or MVISQLMLKLAGDVEENPGP (SEQ ID NO: 59)); porcine teschovirus-1 2A sequence (GSGATNFSLLKQAGDVEENPGP (SEQ ID NO: 60)); and equine rhinitis A virus 2A sequence (GSGQCTNYALLKLAGDVESNPGP (SEQ ID NO: 61)). In some embodiments, the separation sequence is a naturally occurring or synthetic sequence. In some embodiments, the separation sequence includes the 2A consensus sequence D-X-E-X-NPGP (SEQ ID NO: 62), in which X is any amino acid residue.

Alternatively, an Internal Ribosome Entry Site (IRES) may be used to link the CAR a nd the additional gene. IRES is an RNA element that allows for translation initiation in a cap-independent manner. IRES can link two coding sequences in one bicistronic vector and allow the translation of both proteins in cells.

In certain embodiments, the immune effector cells can be genetically modified to express not only CARs as disclosed herein but to also express fusion protein with signaling activity (e.g., costimulation, T-cell activation). These fusion proteins can improve host cell activation and/or responsiveness. In certain embodiments, the fusion protein can enhance the host cell's response to the target antigen. In certain embodiments, the fusion protein can impart resistance to suppression signals.

In certain embodiments, fusion proteins can comprise portions of CD4, CD8a, CD28, portions of a T-cell receptor, or an antigen-binding moiety (e.g., scFv) linked to a MyD88, CD40, and/or other signaling molecules.

In certain embodiments, the fusion protein comprises an extracellular target-binding domain (as disclosed above), a transmembrane domain (as described above) and a cytoplasmic domain, wherein the cytoplasmic domain comprises at least one co-stimulatory protein (as described above). In certain embodiments, the co-stimulatory fusion protein does not comprise a lymphocyte activation domain (e.g., CD3ζ). In certain embodiments, the at least one co-stimulatory protein can be a MyD88 polypeptide or functional fragment thereof, and/or a CD40 cytoplasmic polypeptide region or a functional fragment thereof.

In certain embodiments, the fusion protein comprises an extracellular domain (such as, but not limited to CD19, CD34), a transmembrane domain (as described above) and a cytoplasmic domain, wherein the cytoplasmic domain comprises at least one co-stimulatory protein (as described above). In certain embodiments, the fusion protein does not comprise a lymphocyte activation domain (e.g., CD3ζ). In certain embodiments, the at least one portion of the fusion protein can be a MyD88 polypeptide or functional fragment thereof, and/or a CD40 cytoplasmic polypeptide region or a functional fragment thereof.

Non-limiting examples of fusion proteins include, but are not limited to, the constructs in the publication of WO2019222579 and WO2016073875, which are incorporated herein by reference in their entirety for all purposes.

In certain embodiments, the fusion proteins are introduced into the immune effector cells on a separate vector from the CAR. In certain embodiments, the fusion proteins are introduced into the immune effector cells on the same vector as the CAR. In certain embodiments, the fusion proteins are introduced into the immune effector cells on the same vector as the CAR but separated by a separation sequence such as 2A.

### Methods for Generating Modified Immune Effector Cells

In one aspect, the present invention provides a method for generating a modified immune effector cell described herein. Such method includes deleting or modifying a DNMT3A gene or gene product in the cell so that the DNMT3A-mediated de novo DNA methylation of the cell genome is inhibited.

In some embodiments, the DNMT3A gene in the immune effector cell is deleted or modified as a result of an activity of a site-specific nuclease. The term “site-specific nuclease” as used herein refers to a nuclease capable of specifically recognizing and cleaving a nucleic acid (DNA or RNA) sequence. Suitable site-specific nucleases for use in the present invention include, but are not limited to, RNA-guided endonuclease (e.g., CRISPR-associated (Cas) proteins), zinc finger nuclease, a TALEN nuclease, or mega-TALEN nuclease.

Site-specific nucleases may create double-strand breaks or single-strand breaks (i.e., nick) in a genomic DNA of a cell. Although not wishing to be bound by theory, these breaks are typically repaired by the cell using one of two mechanisms: non-homologous end joining (NHEJ) and homology-directed repair (HDR). In NHEJ, the double-strand breaks are repaired by direct ligation of the break ends to one another. As a result, no new nucleic acid material is inserted into the site, although a few bases may be lost or added, resulting in a small insertions and deletion (indel). In HDR, a donor polynucleotide with homology to the cleaved target DNA sequence is used as a template to repair the cleaved target DNA sequence, resulting in the transfer of genetic information from the donor polynucleotide to the target DNA. As such, new nucleic acid material may be inserted or copied into the cleavage site. In some cases, an exogenous donor polynucleotide can be provided to the cell. The modifications of the target DNA due to NHEJ and/or HDR may lead to, for example, gene correction, gene replacement, gene tagging, transgene insertion, nucleotide deletion, gene disruption, gene mutation, sequence replacement, etc. Accordingly, cleavage of DNA by a site-directed nuclease may be used to delete nucleic acid material from a target DNA sequence by cleaving the target DNA sequence and allowing the cell to repair the sequence in the absence of an exogenously provided donor polynucleotide. Thus, the methods can be used to knock out a gene (resulting in complete lack of transcription or altered transcription) or to knock in genetic material (e.g., a transgene) into a locus of choice in the target DNA.

In some embodiments, the site-specific nuclease is an RNA-guided endonuclease. In particular, a group of RNA-guided endonucleases known as CRISPR-associated (Cas) proteins may be employed to genetically modify the immune effector cell. A Cas protein may form an RNA-protein complex (referred to as RNP) with a guide RNA (gRNA) and is capable of cleaving a target site bearing sequence complementarity to a short sequence (typically about 20-40nt) in the gRNA. In some embodiments, the RNA-guided endonuclease is a Cas9 protein, Cpf1 (Cas12a) protein, C2cl protein, C2c3 protein, or C2c2 protein.

In a specific embodiment, the RNA-guided endonuclease is a Cas9 protein. The Cas9 protein may be from S. pyogenes, Streptococcus thermophilus, Neisseria meningitidis, F. novicida, S. mutans or Treponema denticola. The Cas9 may be a native or modified Cas9 protein.

The Cas9 protein may be programmed with a gRNA that targets a locus with or near the DNMT3A gene. In some embodiments, the gRNA comprises a nucleotide sequence encoded by SEQ ID NO: 63. In some embodiments, the gRNA comprises a nucleotide sequence encoded by SEQ ID NO: 75. In some embodiments, the gRNA comprises a nucleotide sequence encoded by SEQ ID NO: 68. In some embodiments, the gRNA comprises a nucleotide sequence encoded by SEQ ID NO: 76.

In alternative embodiments, the site-specific nuclease used in the methods described herein is a zinc finger nuclease, a TALEN nuclease, or a mega-TALEN nuclease.

In some embodiments, the DNMT3A gene product in the immune effector cell is deleted or modified as a result of an activity of an RNA interference (RNAi) molecule or an antisense oligonucleotide. RNA interference (RNAi) refers to the process of sequence-specific post-transcriptional gene silencing in animals mediated by small interfering RNAs (siRNAs) (Fire et al., 1998, Nature, 391, 806; Hamilton et al., 1999, Science, 286, 950-951). Any small nucleic acid molecules capable of mediating RNAi, such as a short interfering nucleic acid (siNA), a small interfering RNA (siRNA), a double-stranded RNA (dsRNA), a micro-RNA (miRNA), and a short hairpin RNA (shRNA), may be to inhibit the expression of the DNMT3A gene. An antisense oligonucleotide (ASO) is a short nucleotide sequence that can hybridize or bind (e.g., by Watson-Crick base pairing) in a complementary fashion to its target sequence.

In some embodiments, the RNAi molecule is a small interfering RNA (siRNA) or a small hairpin RNA (shRNA). siRNAs, also known as short interfering RNA or silencing RNA, are a class of double-stranded RNA molecules, 20-25 base pairs in length, and operating within the RNA interference (RNAi) pathway. shRNAs or short hairpin RNAs are a group of artificial RNA molecules with a tight hairpin turn that can be used to silence target gene expression via RNA interference (RNAi).

In various embodiments, the site-specific nuclease, the RNAi molecule, or the antisense oligonucleotide as described above is introduced into the immune effector cell via a viral vector, a non-viral vector or a physical means.

The methods for generating a modified immune effector cell described herein may further includes activating the STAT5 signaling pathway in the immune effector cell by a signaling molecule. In some embodiments, the signaling molecule is a common gamma chain cytokine. Non-limiting examples of cytokines that may be used in the methods described herein include IL-15, IL-7, IL-2, IL-4, IL-9, and IL-21.

In some embodiments, the STAT5 signaling pathway is activated by modifying the immune effector cell to express a constitutively active cytokine receptor or a switch receptor. Such constitutively active cytokine receptor may be a constitutively active IL7 receptor (C7R). Such switch receptor may be an IL-4/IL-7 receptor or an IL-4/IL-2 receptor.

In some embodiments, the immune effector cell is contacted with an effective amount of the signaling molecule or a carrier containing the signaling molecule. Suitable carriers include, but are not limited to, polymers, micelles, reverse micelles, liposomes, emulsions, hydrogels, microparticles, nanoparticles, and microspheres. In some embodiments, the carrier is a nanoparticle.

In some embodiments, the immune effector cell is contacted with the signaling molecule more than once. The immune effector cell may be contacted with the signaling molecule 2 times, 3 times, 4 times, 5 times, 6 times, 7 times, 8 times, or more than 8 times. The immune effector cell may be contacted with the signaling molecule at a frequency of every 8 hours, every 12 hours, every 16 hours, every 24 hours, every 2 days, every 3 days, every 4 days, every 5 days, every 6 days, every 7 days, every 8 days, every 8 days, every 10 days, once a week, twice a week, biweekly, once a month, twice a month, 3 times a month, 4 times a month, or 5 times a month.

In some embodiments, the signaling molecule is expressed in the immune effector cell. The signaling molecule may be expressed from a transgene introduced into the immune effector cell. The signaling molecule-expressing transgene may be introduced into the immune effector cell using a viral vector, a non-viral vector or a physical means.

In some embodiments, the modified immune effector cell is further engineered to express a chimeric antigen receptor (CAR) as described herein. The CAR may comprise an extracellular antigen-binding domain, a transmembrane domain, and/or a cytoplasmic domain as described above. The CAR may be expressed from a transgene introduced into the immune effector cell. The CAR-expressing transgene may be introduced into the immune effector cell using a viral vector, a non-viral vector or a physical means.

In some embodiments, the immune effector cells are T cells.

In some embodiments, the immune effector cells are NK cells.

In some embodiments, the immune effector cells are stem cells that are capable of differentiating into immune cells, including induced pluripotent stem cells (iPSCs).

Modified immune effector cells can be activated and/or expanded ex vivo for use in adoptive cellular immunotherapy in which infusions of such cells have been shown to have anti-disease reactivity in a disease-bearing subject. The compositions and methods of this invention can be used to generate a population of immune effector cells (e.g., T lymphocyte or natural killer cells) with enhanced immune cell function for use in immunotherapy in the treatment of the disease.

### Isolation/Enrichment

The immune effector cells may be autologous/autogeneic (“self”) or non-autologous (“non-self,” e.g., allogeneic, syngeneic or xenogeneic). In some embodiments, the immune effector cells are obtained from a mammalian subject. In other embodiments, the immune effector cells are obtained from a primate subject. In some embodiments, the immune effector cells are obtained from a human subject.

Lymphocytes can be obtained from sources such as, but not limited to, peripheral blood mononuclear cells, bone marrow, lymph nodes tissue, cord blood, thymus issue, tissue from a site of infection, ascites, pleural effusion, spleen tissue, and tumors. Lymphocytes may also be generated by differentiation of stem cells. In some embodiments, lymphocytes can be obtained from blood collected from a subject using techniques generally known to the skilled person, such as sedimentation, e.g., FICOLL™ separation.

In some embodiments, cells from the circulating blood of a subject are obtained by apheresis. An apheresis device typically contains lymphocytes, including T cells, monocytes, granulocytes, B cells, other nucleated white blood cells, red blood cells, and platelets. In some embodiments, the cells collected by apheresis may be washed to remove the plasma fraction and to place the cells in an appropriate buffer or media for subsequent processing. The cells can be washed with PBS or with another suitable solution that lacks calcium, magnesium, and most, if not all other, divalent cations. A washing step may be accomplished by methods known to those in the art, such as, but not limited to, using a semiautomated flowthrough centrifuge (e.g., Cobe 2991 cell processor, or the Baxter CytoMate). After washing, the cells may be resuspended in a variety of biocompatible buffers, cell culture medias, or other saline solution with or without buffer.

In some embodiments, immune effector cells can be isolated from peripheral blood mononuclear cells (PBMCs) by lysing the red blood cells and depleting the monocytes. As an example, the cells can be sorted by centrifugation through a PERCOLL™ gradient. In some embodiments, after isolation of PBMC, both cytotoxic and helper T lymphocytes can be sorted into naive, memory, and effector T cell subpopulations either before or after activation, expansion, and/or genetic modification.

In some embodiments, T lymphocytes can be enriched. For example, a specific subpopulation of T lymphocytes, expressing one or more markers such as, but not limited to, CD3, CD4, CD8, CD14, CD15, CD16, CD19, CD27, CD28, CD34, CD3δ, CD45RA, CD45RO, CD56, CD62, CD62L, CD122, CD123, CD127, CD235a, CCR7, HLA-DR or a combination thereofusing either positive or negative selection techniques. In some embodiments, the T lymphocytes for use in the compositions of the invention do not express or do not substantially express one or more of the following markers: CD57, CD244, CD160, PD-1, CTLA4, TIM3, and LAG3.

In some embodiments, NK cells can be enriched. For example, a specific subpopulation of T lymphocytes, expressing one or more markers such as, but not limited to, CD2, CD16, CD56, CD57, CD94, CD122 or a combination thereof using either positive or negative selection techniques.

### Stimulation/Activation

In order to reach sufficient therapeutic doses of immune effector cell compositions, immune effector cells are often subjected to one or more rounds of stimulation/activation. In some embodiments, a method of producing immune effector cells for administration to a subject comprises stimulating the immune effector cells to become activated in the presence of one or more stimulatory signals or agents (e.g., compound, small molecule, e.g., small organic molecule, nucleic acid, polypeptide, or a fragment, isoform, variant, analog, or derivative thereof). In some embodiments, a method of producing immune effector cells for administration to a subject comprises stimulating the immune effector cells to become activated and to proliferate in the presence of one or more stimulatory signals or agents.

Immune effector cells (e.g., T lymphocytes and NK cells) can be activated by inducing a change in their biologic state by which the cells express activation markers, produce cytokines, proliferate and/or become cytotoxic to target cells. All these changes can be produced by primary stimulatory signals. Co-stimulatory signals amplify the magnitude of the primary signals and suppress cell death following initial stimulation resulting in a more durable activation state and thus a higher cytotoxic capacity.

T cells can be activated generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In some embodiments, the T cell based immune effector cells can be activated by binding to an agent that activates CD3ζ.

In other embodiments, a CD2-binding agent may be used to provide a primary stimulation signal to the T cells. For example, and not by limitation, CD2 agents include, but are not limited to, CD2 ligands and anti-CD2 antibodies, e.g., the Tl 1.3 antibody in combination with the Tl 1.1 or Tl 1.2 antibody (Meuer, S. C. et al. (1984) Cell 36:897-906) and the 9.6 antibody (which recognizes the same epitope as TI 1.1) in combination with the 9-1 antibody (Yang, S. Y. et al. (1986) J. Immunol. 137:1097-1100, which is incorporated herein by reference in its entirety). Other antibodies which bind to the same epitopes as any of the above described antibodies can also be used.

In some embodiments, the immune effector cells are activated by administering phorbol myristate acetate (PMA) and ionomycine. In some embodiments, the immune effector cells are activated by administering an appropriate antigen that induces activation and then expansion. In some embodiments, PMA, ionomycin, and/or appropriate antigen are administered with CD3 induce activation and/or expansion.

In general, the activating agents used in the present invention includes, but is not limited to, an antibody, a fragment thereof and a proteinaceous binding molecule with antibody-like functions. Examples of (recombinant) antibody fragments are Fab fragments, Fv fragments, single-chain Fv fragments (scFv), a divalent antibody fragment such as an (Fab)2′-fragment, diabodies, triabodies (Iliades, P., et al., FEBS Lett (1997) 409, 437-441, which is incorporated herein by reference in its entirety), decabodies (Stone, E., et al., Journal of Immunological Methods (2007) 318, 88-94, which is incorporated herein by reference in its entirety) and other domain antibodies (Holt, L. J., et al., Trends Biotechnol. (2003), 21, 11, 484-490, which is incorporated herein by reference in its entirety). The divalent antibody fragment may be an (Fab)2′-fragment, or a divalent single-chain Fv fragment while the monovalent antibody fragment may be selected from the group consisting of a Fab fragment, a Fv fragment, and a single-chain Fv fragment (scFv).

In some embodiments, one or more binding sites of the CD3ζ agents may be a bivalent proteinaceous artificial binding molecule such as a dimeric lipocalin mutein (i.e., duocalin). In some embodiments the receptor binding reagent may have a single second binding site, (i.e., monovalent). Examples of monovalent agents include, but are not limited to, a monovalent antibody fragment, a proteinaceous binding molecule with antibody-like binding properties or an MHC molecule. Examples of monovalent antibody fragments include, but are not limited to a Fab fragment, a Fv fragment, and a single-chain Fv fragment (scFv), including a divalent single-chain Fv fragment.

The agent that specifically binds CD3 includes, but is not limited to, an anti-CD3-antibody, a divalent antibody fragment of an anti-CD3 antibody, a monovalent antibody fragment of an anti-CD3-antibody, and a proteinaceous CD3-binding molecule with antibody-like binding properties. A proteinaceous CD3-binding molecule with antibody-like binding properties can be an aptamer, a mutein based on a polypeptide of the lipocalin family, a glubody, a protein based on the ankyrin scaffold, a protein based on the crystalline scaffold, an adnectin, and an avimer. It also can be coupled to a bead.

In some embodiments, the activating agent (e.g., CD3-binding agents) can be present in a concentration of about 0.1 to about 10 ag/ml. In some embodiments, the activating agent (e.g., CD3-binding agents) can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 g/ml, or about 0.9 μg/ml to about 2 μg/ml. In some embodiments, the activating agent (e.g., CD3-binding agents) is administered at a concentration of about 0.1 μg/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In some embodiments, the CD3—binding agents can be present in a concentration of 1 μg/ml.

NK cells can be activated generally using methods as described, for example, in U.S. Pat. Nos. 7,803,376, 6,949,520, 6,693,086, 8,834,900, 9,404,083, 9,464,274, 7,435,596, 8,026,097, and 8,877,182; U.S. Patent Applications US2004/0058445, US2007/0160578, US2013/0011376, US2015/0118207, and US2015/0037887; and PCT Patent Application WO2016/122147, each of which is incorporated herein by reference in its entirety.

In some embodiments, the NK based immune effector cells can be activated by, for example and not limitation, inhibition of inhibitory receptors on NK cells (e.g., KIR2DL1, KIR2DL2/3, KIR2DL4, KIR2DL5A, KIR2DL5B, KIR3DL1, KIR3DL2, KIR3DL3, LILRB1, NKG2A, NKG2C, NKG2E or LTLRB5 receptor).

In some embodiments, the NK based immune effector cells can be activated by, for example and not limitation, feeder cells (e.g., native K562 cells or K562 cells that are genetically modified to express 4-1BBL and cytokines such as IL15 or IL21).

In other embodiments, interferons or macrophage-derived cytokines can be used to activate NK cells. For example and not limitation, such interferons include but are not limited to interferon alpha and interferon gamma, and such cytokines include but are not limited to TL-15, IL-2, IL-21.

In some embodiments, the NK activating agent can be present in a concentration of about 0.1 to about 10 μg/ml. In some embodiments, the NK activating agent can be present in a concentration of about 0.2 μg/ml to about 9 μg/ml, about 0.3 μg/ml to about 8 μg/ml, about 0.4 μg/ml to about 7 μg/ml, about 0.5 μg/ml to about 6 μg/ml, about 0.6 μg/ml to about 5 μg/ml, about 0.7 μg/ml to about 4 μg/ml, about 0.8 μg/ml to about 3 μg/ml, or about 0.9 μg/ml to about 2 ag/ml. In some embodiments, the NK activating agent is administered at a concentration of about 0.1 g/ml, about 0.2 μg/ml, about 0.3 μg/ml, about 0.4 μg/ml, about 0.5 μg/ml, about 0.6 μg/ml, about 0.7 μg/ml, about 0.8 μM, about 0.9 μg/ml, about 1 μg/ml, about 2 μg/ml, about 3 μg/ml, about 4 μM, about 5 μg/ml, about 6 μg/ml, about 7 μg/ml, about 8 μg/ml, about 9 μg/ml, or about 10 μg/ml. In some embodiments, the NK activating agent can be present in a concentration of 1 μg/ml.

In some embodiments, the activating agent is attached to a solid support such as, but not limited to, a bead, an absorbent polymer present in culture plate or well or other matrices such as, but not limited to, Sepharose or glass; may be expressed (such as in native or recombinant forms) on cell surface of natural or recombinant cell line by means known to those skilled in the art.

Polynucleotide and/or Polypeptide Transfer

In some embodiments, the immune effector cells are genetically modified to by introducing polynucleotides and/or polypeptide (e.g., a CAR, a signaling molecule, site-specific nuclease, an RNAi molecule or an antisense oligonucleotide, or polynucleotides encoding the same). The immune effector cells can be genetically modified after stimulation/activation. In some embodiments, the immune effector cells are modified within 12 hours, 16 hours, 24 hours, 36 hours, or 48 hours of stimulation/activation. In some embodiments, the cells are modified within 16 to 24 hours after stimulation/activation. In some embodiments, the immune effector cells are modified within 24 hours.

In order to genetically modify the immune effector cell, the polynucleotides and/or polypeptide (e.g., a CAR, a signaling molecule, site-specific nuclease, an RNAi molecule or an antisense oligonucleotide, or polynucleotides encoding the same) must be transferred into the host cell. Polynucleotide and/or polypeptide transfer may be via viral, non-viral gene delivery methods, or a physical method. Suitable methods for polynucleotide and/or polypeptide delivery for use with the current methods include any method known by those of skill in the art, by which a polynucleotide and/or polypeptide can be introduced into an organelle, cell, tissue or organism.

In various embodiments, polypeptides or polynucleotides (e.g., a CAR, a signaling molecule, site-specific nuclease, an RNAi molecule or an antisense oligonucleotide, or polynucleotides encoding the same) described in the present invention are introduced to the immune effector cell via a recombinant vector.

In some embodiments, the vector is a viral vector. Suitable viral vectors that can be used in the present invention include, but are not limited to, a retroviral vector, an adenoviral vector, an adeno-associated viral (AAV) vector, a herpes viral vector, or a baculoviral vector. In one specific embodiment, the viral vector is a lentiviral vector.

In some embodiments, the immune effector cells can be transduced via retroviral transduction. References describing retroviral transduction of genes are Anderson et al., U.S. Pat. No. 5,399,346; Mann et al., Cell 33:153 (1983); Temin et al., U.S. Pat. No. 4,650,764; Temin et al., U.S. Pat. No. 4,980,289; Markowitz et al., J. Virol. 62:1120 (1988); Temin et al., U.S. Pat. No. 5,124,263; International Patent Publication No. WO 95/07358, published Mar. 16, 1995, by Dougherty et al.; and Kuo et al., Blood 82:845 (1993), each of which is incorporated herein by reference in its entirety.

One method of genetic modification includes ex vivo modification. Various methods are available for transfecting cells and tissues removed from a subject via ex vivo modification. For example, retroviral gene transfer in vitro can be used to genetically modified cells removed from the subject and the cell transferred back into the subject. See e.g., Wilson et al., Science, 244:1344-1346, 1989 and Nabel et al., Science, 244(4910):1342-1344, 1989, both of which are incorporated herein by reference in their entity. In some embodiments, the immune effector cells may be removed from the subject and transfected ex vivo using the polynucleotides (e.g., expression vectors) of the invention. In some embodiments, the immune effector cells obtained from the subject can be transfected or transduced with the polynucleotides (e.g., expression vectors) of the invention and then administered back to the subject.

In some embodiments, polynucleotides and/or polypeptides are transferred to the cell in a non-viral vector. In some embodiments, the non-viral vector is a transposon. Exemplary transposons that can be used in the present invention include, but are not limited to, a sleeping beauty transposon and a PiggyBac transposon.

Nucleic acid vaccines may also be used to transfer polynucleotides into the immune effector cells. Such vaccines include, but are not limited to non-viral polynucleotide vectors, “naked” DNA and RNA, and viral vectors. Methods of genetically modifying cells with these vaccines, and for optimizing the expression of genes included in these vaccines are known to those of skill in the art.

In some embodiments, the polynucleotide(s) is operatively linked to at least one regulatory element for expression of the gene product (e.g., a CAR, a signaling molecule, site-specific nuclease, an RNAi molecule). The regulatory element can be capable of mediating expression of the gene product in the host cell (e.g., modified immune effector cell). Regulatory elements include, but are not limited to, promoters, enhancers, initiation sites, polyadenylation (polyA) tails, IRES elements, response elements, and termination signals. In some embodiments, the regulatory element regulates expression of the gene product. In some embodiments, the regulatory element increased the expression of the gene product. In some embodiments, the regulatory element increased the expression of the gene product once the host cell (e.g., modified immune effector cell) is activated. In some embodiments, the regulatory element decreases expression of the gene product. In some embodiments, the regulatory element decreases expression of the gene product once the host cell (e.g., modified immune effector cell) is activated.

In various embodiment, polypeptides or polynucleotides (e.g., a CAR, a signaling molecule, site-specific nuclease, an RNAi molecule or an antisense oligonucleotide, or polynucleotides encoding the same) are introduced into the modified immune effector cell using a physical means. Suitable physical means include, but are not limited to, electroporation, microinjection, magnetofection, ultrasound, a ballistic or hydrodynamic method, or a combination thereof.

Electroporation is a method for polynucleotide and/or polypeptide delivery. See e.g., Potter et al., (1984) Proc. Nat'l Acad. Sci. USA, 81, 7161-7165 and Tur-Kaspa et al., (1986) Mol. Cell Biol., 6, 716-718, both of which are incorporated herein in their entirety for all purposes. Electroporation involves the exposure of a suspension of cells and DNA to a high-voltage electric discharge. In some embodiments, cell wall-degrading enzymes, such as pectin-degrading enzymes, can be employed to render the immune effector cells more susceptible to genetic modification by electroporation than untreated cells. See e.g., U.S. Pat. No. 5,384,253, incorporated herein by reference in its entirety for all purposes.

In vivo electroporation involves a basic injection technique in which a vector is injected intradermally in a subject. Electrodes then apply electrical pulses to the intradermal site causing the cells localized there (e.g., resident dermal dendritic cells), to take up the vector. These tumor antigen-expressing dendritic cells activated by local inflammation can then migrate to lymph-nodes.

Methods of electroporation for use with this invention include, for example, Sardesai, N. Y., and Weiner, D. B., Current Opinion in Immunotherapy 23:421-9 (2011) and Ferraro, B. et al., Human Vaccines 7:120-127 (2011), both of which are hereby incorporated by reference herein in their entirety for all purposes.

Another method for polynucleotide and/or polypeptide transfer includes injection. In some embodiments, a polypeptide, a polynucleotide or viral vector may be delivered to a cell, tissue, or organism via one or more injections (e.g., a needle injection). Non-limiting methods of injection include injection of a composition (e.g., a saline based composition). Polynucleotides and/or polynucleotides can also be introduced by direct microinjection. Non-limiting sites of injection include, subcutaneous, intradermal, intramuscular, intranodal (allows for direct delivery of antigen to lymphoid tissues). intravenous, intraprostatic, intratumor, intralymphatic (allows direct administration of DCs) and intraperitoneal. It is understood that proper site of injection preparation is necessary (e.g., shaving of the site of injection to observe proper needle placement).

Additional methods of polynucleotide and/or polypeptide transfer include liposome-mediated transfection (e.g., polynucleotide entrapped in a lipid complex suspended in an excess of aqueous solution. See e.g., Ghosh and Bachhawat, (1991) In: Liver Diseases, Targeted Diagnosis and Therapy Using Specific Receptors and Ligands. pp. 87-104). Also contemplated is a polynucleotide and/or polypeptide complexed with Lipofectamine, or Superfect); DEAE-dextran (e.g., a polynucleotide is delivered into a cell using DEAE-dextran followed by polyethylene glycol. See e.g., Gopal, T. V., Mol Cell Biol. 1985 May; 5(5):1188-90); calcium phosphate (e.g., polynucleotide is introduced to the cells using calcium phosphate precipitation. See e.g., Graham and van der Eb, (1973) Virology, 52, 456-467; Chen and Okayama, Mol. Cell Biol., 7(8):2745-2752, 1987), and Rippe et al., Mol. Cell Biol., 10:689-695, 1990); sonication loading (introduction of a polynucleotide by direct sonic loading. See e.g., Fechheimer et al., (1987) Proc. Nat'l Acad. Sci. USA, 84, 8463-8467); microprojectile bombardment (e.g., one or more particles may be coated with at least one polynucleotide and/or polypeptide and delivered into cells by a propelling force. See e.g., U.S. Pat. Nos. 5,550,318; 5,538,880; 5,610,042; and PCT Application WO 94/09699; Klein et al., (1987) Nature, 327, 70-73, Yang et al., (1990) Proc. Nat'l Acad. Sci. USA, 87, 9568-9572); and receptor-mediated transfection (e.g., selective uptake of macromolecules by receptor-mediated endocytosis that will be occurring in a target cell using cell type-specific distribution of various receptors. See e.g., Wu and Wu, (1987) J. Biol. Chem., 262, 4429-4432; Wagner et al., Proc. Natl. Acad. Sci. USA, 87(9):3410-3414, 1990; Perales et al., Proc. Natl. Acad. Sci. USA, 91:4086-4090, 1994; Myers, EPO 0273085; Wu and Wu, Adv. Drug Delivery Rev., 12:159-167, 1993; Nicolau et al., (1987) Methods Enzymol., 149, 157-176), each reference cited here is incorporated by reference in their entirety for all purposes. Expansion/Proliferation

After the immune effector cells are activated and transduced, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of T cells can include interleukins, such as IL-2, IL-7, IL-15, or IL-21 (see for example Cornish et al. 2006, Blood. 108(2):600-8, Bazdar and Sieg, 2007, Journal of Virology, 2007, 81(22):12670-12674, Battalia et al, 2013, Immunology, 139(1):109-120, each of which is incorporated by reference in their entirety for all purposes). Other illustrative examples for agents that may be used for the expansion of T cells are agents that bind to CD8, CD45 or CD90, such as αCD8, αCD45 or αCD90 antibodies. Illustrative examples of T cell population including antigen-specific T cells, T helper cells, cytotoxic T cells, memory T cell (an illustrative example of memory T cells are CD62L+CD8+ specific central memory T cells) or regulatory T cells (an illustrative example of Treg are CD4+CD25+CD45RA+ Treg cells).

Additional agents that can be used to expand T lymphocytes includes methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety.

In some embodiments, the agent(s) used for expansion (e.g., IL-7, IL-15) are administered at about 20 units/ml to about 200 units/ml. In some embodiments, the agent(s) used for expansion (e.g., IL-7, IL-15) are administered at about 25 units/ml to about 190 units/ml, about 30 units/ml to about 180 units/ml, about 35 units/ml to about 170 units/ml, about 40 units/ml to about 160 units/ml, about 45 units/ml to about 150 units/ml, about 50 units/ml to about 140 units/ml, about 55 units/ml to about 130 units/ml, about 60 units/ml to about 120 units/ml, about 65 units/ml to about 110 units/ml, about 70 units/ml to about 100 units/ml, about 75 units/ml to about 95 units/ml, or about 80 units/ml to about 90 units/ml. In some embodiments, the agent(s) used for expansion (e.g., IL-7, IL-15) are administered at about 20 units/ml, about 25 units/ml, about 30 units/ml, 35 units/ml, 40 units/ml, 45 units/ml, about 50 units/ml, about 55 units/ml, about 60 units/ml, about 65 units/ml, about 70 units/ml, about 75 units/ml, about 80 units/ml, about 85 units/ml, about 90 units/ml, about 95 units/ml, about 100 units/ml, about 105 units/ml, about 110 units/ml, about 115 units/ml, about 120 units/ml, about 125 units/ml, about 130 units/ml, about 135 units/ml, about 140 units/ml, about 145 units/ml, about 150 units/ml, about 155 units/ml, about 160 units/ml, about 165 units/ml, about 170 units/ml, about 175 units/ml, about 180 units/ml, about 185 units/ml, about 190 units/ml, about 195 units/ml, or about 200 units/ml. In some embodiments, the agent(s) used for expansion (e.g., IL-7, IL-15) are administered at about 5 mg/ml to about 10 ng/ml. In some embodiments, the agent(s) used for expansion (e.g., IL-7, IL-15) are administered at about 5.5 ng/ml to about 9.5 ng/ml, about 6 ng/ml to about 9 ng/ml, about 6.5 ng/ml to about 8.5 ng/ml, or about 7 ng/ml to about 8 ng/ml. In some embodiments, the agent(s) used for expansion (e.g., IL-7, IL-15) are administered at about 5 ng/ml, 6 ng/ml, 7 ng/ml, 8 ng/ml, 9, ng/ml, or 10 ng/ml.

After the immune effector cells are activated and transduced, the cells are cultured to proliferate. NK cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion.

Agents that can be used for the expansion of natural killer cells can include agents that bind to CD16 or CD56, such as for example αCD16 or αCD56 antibodies. In some embodiments, the binding agent includes antibodies (see for example Hoshino et al, Blood. 1991 Dec. 15; 78(12):3232-40.). Other agents that may be used for expansion of NK cells may be IL-15 (see for example Vitale et al. 2002. The Anatomical Record. 266:87-92, which is incorporated by reference in their entirety for all purposes).

Conditions appropriate for T cell culture include an appropriate media (e.g., Minimal Essential Media (MEM), RPMI Media 1640, Lonza RPMI 1640, Advanced RPMI, Clicks, AIM-V, DMEM, a-MEM, F-12, TexMACS, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion).

Examples of other additives for immune effector cell expansion include, but are not limited to, surfactant, piasmanate, pH buffers such as HEPES, and reducing agents such as N-acetyl-cysteine and 2-mercaptoethanol, Antibiotics (e.g., penicillin and streptomycin), are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In certain embodiments, host cells of the present disclosure may be modified such that the expression of an endogenous TCR, MHC molecule, or other immunogenic molecule is decreased or eliminated. When allogeneic cells are used, rejection of the therapeutic cells may be a concern as it may cause serious complications such as the graft-versus-host disease (GvHD). Although not wishing to be bound by theory, immunogenic molecules (e.g., endogenous TCRs and/or MHC molecules) are typically expressed on the cell surface and are involved in self vs non-self-discrimination. Decreasing or eliminating the expression of such molecules may reduce or eliminate the ability of the therapeutic cells to cause GvHD.

In certain embodiments, expression of an endogenous TCR in the host cells is decreased or eliminated. In a particular embodiment, expression of an endogenous TCR (e.g., ap TCR) in the host cells is decreased or eliminated. Expression of the endogenous TCR may be decreased or eliminated by disrupting the TRAC locus, TCR beta constant locus, and/or CD3 locus. In certain embodiments, expression of an endogenous TCR may be decreased or eliminated by disrupting one or more of the TRAC, TRBC1, TRBC2, CD3E, CD3G, and/or CD3D locus.

In certain embodiments, expression of one or more endogenous MHC molecules in the host cells is decreased or eliminated. Modified MHC molecule may be an MHC class I or class II molecule. In certain embodiments, expression of an endogenous MHC molecule may be decreased or eliminated by disrupting one or more of the MHC, βM, TAP1, TAP2, CIITA, RFX5, RFXAP and/or RFXANK locus.

Expression of the endogenous TCR, an MHC molecule, and/or any other immunogenic molecule in the host cell can be disrupted using genome editing techniques such as Clustered regularly interspaced short palindromic repeats (CRISPR)/Cas, zinc finger nucleases (ZFNs), transcription activator-like effector nucleases (TALENs), and Meganucleases. These genome editing methods may disrupt a target gene by entirely knocking out all of its output or partially knocking down its expression. In a particular embodiment, expression of the endogenous TCR, an MHC molecule and/or any other immunogenic molecule in the host cell is disrupted using the CRISPR/Cas technique.

### Methods of Preserving Immune Effector Cell Function

In one aspect, the present invention provides a method of preserving an immune effector cell, including deleting or modifying a DNA (cytosine-5)-methyltransferase 3A (DNMT3A) gene or gene product in the cell so that the DNMT3A-mediated de novo DNA methylation of the cell genome is inhibited; and, enhancing IL10 signaling pathway in the immune effector cell.

In some embodiments, the immune effector cell is a T cell. Non-limiting examples of a T cell are a CD8+ T cell, a CD4+ T cell, a cytotoxic T cell, an αβ T cell receptor (TCR) T cell, a natural killer T (NKT) cell, a γδ T cell, a memory T cell, a T-helper cell, and a regulatory T cell (Treg). In some embodiments, the immune effector cell is a NK cell.

In some embodiments, the DNMT3A gene in the immune effector cell is deleted or modified as a result of an activity of a site-specific nuclease. In some embodiments, the site-specific nuclease is an RNA-guided endonuclease. Non-limiting examples of an RNA-guided nuclease include, but are not limited to, Cas9 protein, Cpf1 (Cas12a) protein, C2c1 protein, C2c3 protein, and C2c2 protein. In some embodiments, the RNA-guided endonuclease is a Cas9 protein. In some embodiments, the RNA-guided endonuclease is a Cpf1 (Cas12a) protein. In some embodiments, the RNA-guided endonuclease is a C2cl protein. In some embodiments, the RNA-guided endonuclease is a C2c3 protein, In some embodiments, the RNA-guided endonuclease is a C2c2 protein.

In some embodiments, the RNA-guided endonuclease is a Cas9 protein, and the Cas9 protein is programmed with a guide RNA (gRNA) targeting DNMT3A.

In some embodiments, the RNA-guided endonuclease is a Cas9 protein, wherein the Cas9 protein is programmed with a guide RNA (gRNA) that comprises a nucleotide sequence encoded by CCTGCATGATGCGCGGCCCANGG (SEQ ID NO: 63). In some embodiments, the guide RNA (gRNA) comprises a nucleotide sequence encoded by a nucleotide sequence at least about 80%, 85%, 90% or 95% identical to SEQ ID NO: 63.

In some embodiments, the RNA-guided endonuclease is a Cas9 protein, wherein the Cas9 protein is programmed with a guide RNA (gRNA) that comprises a nucleotide sequence encoded by CCTGCATGATGCGCGGCCCA (SEQ ID NO: 75). In some embodiments, the guide RNA (gRNA) comprises a nucleotide sequence encoded by a nucleotide sequence at least about 80%, 85%, 90% or 95% identical to SEQ ID NO: 75.

In some embodiments, the RNA-guided endonuclease is a Cas9 protein, wherein the Cas9 protein is programmed with a guide RNA (gRNA) that comprises a nucleotide sequence encoded by GCATGATGCGCGGCCCAAGGNGG (SEQ ID NO: 68). In some embodiments, the guide RNA (gRNA) comprises a nucleotide sequence encoded by a nucleotide sequence at least about 80%, 85%, 90% or 95% identical to SEQ ID NO: 68.

In some embodiments, the RNA-guided endonuclease is a Cas9 protein, wherein the Cas9 protein is programmed with a guide RNA (gRNA) that comprises a nucleotide sequence encoded by GCATGATGCGCGGCCCAAGG (SEQ ID NO: 76). In some embodiments, the guide RNA (gRNA) comprises a nucleotide sequence encoded by a nucleotide sequence at least about 80%, 85%, 90% or 95% identical to SEQ ID NO: 76.

In alternative embodiments, the site-specific nuclease used in the methods described herein is a zinc finger nuclease, a TALEN nuclease, or a mega-TALEN nuclease.

In some embodiments, the DNMT3A gene product in the immune effector cell is deleted or modified as a result of an activity of an RNA interference (RNAi) molecule or an antisense oligonucleotide. In some embodiments, the RNAi molecule is a small interfering RNA (siRNA) or a small hairpin RNA (shRNA).

In various embodiments, the site-specific nuclease, the RNAi molecule or the antisense oligonucleotide as described above is introduced into the immune effector cell via a viral vector, a non-viral vector or a physical means described herein.

In some embodiments, the level of DNMT3A-mediated de novo DNA methylation of the cell genome is inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level of functional DNMT3A protein in the cell may be decreased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the level of DNMT3A-mediated de novo DNA methylation of the cell genome is inhibited by about 50% or more. In some embodiments, the level of DNMT3A-mediated de novo DNA methylation of the cell genome is inhibited by about 70% or more. In some embodiments, the level of DNMT3A-mediated de novo DNA methylation of the cell genome is inhibited by about 90% or more. In some embodiments, the level of DNMT3A-mediated de novo DNA methylation of the cell genome is inhibited by about 99% or more.

In some embodiments, the immune effector cell is contacted with an effective amount of the signaling molecule or a carrier containing the signaling molecule. Suitable carriers include, but are not limited to, polymers, micelles, reverse micelles, liposomes, emulsions, hydrogels, microparticles, nanoparticles, and microspheres. In some embodiments, the carrier is a nanoparticle. In some embodiments, the signaling molecule enhances the I1 signaling pathway. In some embodiments, the signaling molecule is IL10. The IL10 may be an exogenous IL10. The exogenous IL10 may be a recombinant IL10. In some embodiments, the signaling molecule activates the STATS signaling pathway.

In some embodiments, the signaling pathway may be enhanced or activated in any of the immune effector cells of the present disclosure by either stimulating the immune effector cell with a signaling molecule or genetically modifying the immune effector cell to express a signaling molecule using any of the methods disclosed herein.

Enhancing or activating the signaling pathway in the immune effector cell may be achieved by stimulating the immune effector cell with a signaling molecule either ex vivo or in vivo. For example, stimulating the immune effector cell with a signaling molecule may be carried out by mixing the immune effector cell directly with the signaling molecule, or with a carrier (e.g., nanoparticles) containing the signaling molecule ex vivo. Mixing of the immune effector cell with the signaling molecule, or with a carrier (e.g., nanoparticles) containing the signaling molecule may be carried out prior to administration, or during administration. In some embodiments, the immune effector cells may be administered with nanoparticle “backpacks” which are capable of carrying signaling molecules and attaching them to the immune effector cells. Such nanoparticle “backpacks” may selectively release the signaling molecules in response to certain stimuli, such as the activation of the immune effector cell (Tang L., Nat Biotechnol. 2018; 36(8):707-716, which is incorporated by reference in their entirety for all purposes).

Alternatively, signaling molecules may be provided to the modified immune effector cells in vivo by administration of the signaling molecule, for example systemically, to the subject such that the signaling molecule can ultimately contact the modified immune effector cells. Signaling molecules may also be provided to the modified immune effector cells in vivo using oncolytic viruses encoding the signaling molecule. Oncolytic viruses can selectively infect and/or lyse cancer or tumor cells as compared to normal cells. Exemplary oncolytic viruses include herpes simplex virus-1, herpes simplex virus-2, a vesicular stomatitis virus, and a vaccinia virus.

Enhancing or activating the signaling pathway in the immune effector cell may also be achieved by genetically modifying the immune effector cell to express a signaling molecule. The signaling molecule may be expressed from a transgene introduced into the immune effector cell. Alternatively, the signaling pathway is activated by modifying the immune effector cell to express a constitutively active cytokine receptor or a switch receptor. Such constitutively active cytokine receptor may be a constitutively active TL7 receptor (C7R). Such switch receptor may be an IL-4/IL-7 receptor or an TL-4/TL-2 receptor

In some embodiments, the immune effector cell is contacted with the signaling molecule more than once. The immune effector cell may be contacted with the signaling molecule 2 times, 3 times, 4 times, 5 times, 6 times, 7 times, 8 times, or more than 8 times. The immune effector cell may be contacted with the signaling molecule at a frequency of every 8 hours, every 12 hours, every 16 hours, every 24 hours, every 2 days, every 3 days, every 4 days, every 5 days, every 6 days, every 7 days, every 8 days, every 8 days, every 10 days, once a week, twice a week, biweekly, once a month, twice a month, 3 times a month, 4 times a month, or 5 times a month. In some embodiments, the contacting may be conducted ex vivo. In some embodiments, the contacting may be conducted in vivo.

In some embodiments, the signaling molecule is expressed in the immune effector cell. The signaling molecule may be expressed from a transgene introduced into the immune effector cell. The signaling molecule-expressing transgene may be introduced into the immune effector cell using a viral vector, a non-viral vector or a physical means described herein.

In some embodiments, the IL10 signaling pathway in the immune effector cell is enhanced by subjecting the immune effector cell to an effective amount of exogenous IL10. In some embodiments, the IL10 signaling pathway in the immune effector cell is enhanced by subjecting the immune effector cell to a carrier comprising the exogenous IL10. In some embodiments, the exogenous IL10 is a recombinant IL10. In some embodiments, the carrier is a nanoparticle.

In some embodiments, the IL10 signaling pathway is enhanced by genetically modifying the immune effector cell to express IL10 using any of the methods useful for genetic modification of cells disclosed herein. As a non-limiting example the IL10 is expressed form transgene introduced into the immune effector cell. The IL10-expressing transgene may be introduced into the immune effector cell using a viral vector. The IL10-expressing transgene may be introduced into the immune effector cell using a non-viral vector. The IL10-expressing transgene may be introduced into the immune effector cell using physical means.

In certain embodiments, any of the methods for preserving multipotency of an immune effector cell described above may further comprise activating and/or expanding of the immune effector cell. The immune effector cell may be subjected to a signaling molecule, e.g. IL10 such as, but not limited to, exogenous IL10, at the beginning of the expansion.

In some embodiments, the immune effector cell may be subjected to exogenous IL10 at the beginning of the expansion. In some embodiments, the immune effector cell may be subjected to the exogenous IL10 more than once. The immune effector cell may be contacted with the exogenous IL10 2 times, 3 times, 4 times, 5 times, 6 times, 7 times, 8 times, or more than 8 times. The immune effector cell may be contacted with the exogenous IL,10 at a frequency of every 8 hours, every 12 hours, every 16 hours, every 24 hours, every 2 days, every 3 days, every 4 days, every 5 days, every 6 days, every 7 days, every 8 days, every 8 days, every 10 days, once a week, twice a week, biweekly, once a month, twice a month, 3 times a month, 4 times a month, or 5 times a month.

In some embodiments, the subjecting may be conducted ex vivo. In some embodiments, the subjecting may be conducted in vivo.

In some embodiments, the immune effector cell may comprise at least one surface molecule capable of binding specifically to an antigen. In some embodiments, the cell may comprise a chimeric antigen receptor (CAR). In some embodiments, the cell may comprise an antigen specific T-cell receptor (TCR). In some embodiments, the cell may comprise a bispecific antibody. In some embodiments, the cell may comprise a T cell antigen coupler (TAC). In some embodiments, the CAR, TCR, bispecific antibody and/or TAC is expressed from a transgene introduced into the immune effector cell. The CAR, TCR, bispecific antibody and/or TAC may be expressed from a transgene introduced into the immune effector cell using a viral vector, a non-viral vector or a physical means.

Non-limiting examples of a viral vectors that may be useful in any of the methods for preserving immune effector cell function disclosed herein are an adenoviral vector, an adeno-associated viral (AAV) vector, a herpes viral vector, or a baculoviral vector. In some embodiments, the retroviral vector is a lentiviral vector. In some embodiments, the non-viral vector is a transposon such as, but not limited to a sleeping beauty transposon or a PiggyBac transposon.

In some embodiments, the physical means is electroporation, microinjection, magnetofection, ultrasound, a ballistic or hydrodynamic method, or a combination thereof.

In some embodiments, the method further includes genetically modifying the immune effector cell to express a chimeric antigen receptor (CAR) that is capable of binding to an antigen specific for the tumor. Non-limiting examples of the CARs include any of those described herein.

In some embodiments, the method further includes activation and/or expansion of the immune effector cell ex vivo.

### Pharmaceutical Compositions

In some embodiments, the compositions comprise one or more polypeptides, polynucleotides, vectors comprising same, and cell compositions, as disclosed herein. Compositions include, but are not limited to pharmaceutical compositions. In some embodiments, the compositions of the present invention comprise an amount of modified immune effector cells manufactured by the methods disclosed herein.

In one aspect, the present invention provides a pharmaceutical composition comprising a modified immune effector cell described herein and a pharmaceutically acceptable carrier and/or excipient. Examples of pharmaceutical carriers include but are not limited to sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions.

Compositions comprising modified immune effector cells disclosed herein may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

Compositions comprising modified immune effector cells disclosed herein may comprise one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In some embodiments, the compositions are formulated for parenteral administration, e.g., intravascular (intravenous or intraarterial), intraperitoneal, intratumoral, intraventricular, intrapleural or intramuscular administration. The parenteral preparation can be enclosed in ampoules, disposable syringes or multiple dose vials made of glass or plastic. An injectable pharmaceutical composition is preferably sterile. In some embodiments, the composition is reconstituted from a lyophilized preparation prior to administration.

In some embodiments, the modified immune effector cells may be mixed with substances that adhere or penetrate then prior to their administration, e.g., but not limited to, nanoparticles.

### Therapeutic Methods

In one aspect, the present invention provides a method of treating a disease or disorder in a subject in need thereof, including administering to the subject an effective amount of the immune effector cells, e.g., modified immune effector cells, or the pharmaceutical composition described herein. In some embodiments, the modified immune effector cells are prepared by the methods as disclosed above. In some embodiments, the subject is human.

In some embodiments, the immune effector cell is an autologous cell. In some embodiments, the immune effector cell is an allogeneic cell. In some embodiments, the immune effector cell is isolated from a subject having a disease. In some embodiments, the disease is a cancer, an infectious disease an inflammatory disorder, or an autoimmune disorder.

In some embodiments, the immune effector cell is derived from blood, marrow, tissue, or tumor sample.

In some embodiments, the disease being treated by the therapeutic methods described herein is a cancer, an infectious disease, an inflammatory disorder, or an autoimmune disease.

The terms “cancer” and “cancerous” refer to or describe the physiological condition in mammals that is typically characterized by unregulated cell growth. The term “cancer” includes, for example, the soft tissue tumors (e.g., lymphomas), and tumors of the blood and blood-forming organs (e.g., leukemias), and solid tumors, which is one that grows in an anatomical site outside the bloodstream (e.g., carcinomas). Examples of cancer include, but are not limited to, carcinoma, lymphoma, blastoma, sarcoma (e.g., osteosarcoma or rhabdomyosarcoma), and leukemia or lymphoid malignancies. More particular examples of such cancers include squamous cell cancer (e.g., epithelial squamous cell cancer), adenosquamous cell carcinoma, lung cancer (e.g., including small-cell lung cancer, non-small cell lung cancer, adenocarcinoma of the lung, squamous carcinoma of the lung), cancer of the peritoneum, hepatocellular cancer, gastric or stomach cancer (e.g., including gastrointestinal cancer, pancreatic cancer), cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, primary or metastatic melanoma, multiple myeloma and B-cell lymphoma, non-Hodgkin's lymphoma, Hodgkin's lymphoma, brain (e.g., high grade glioma, diffuse pontine glioma, ependymoma, neuroblastoma, or glioblastoma), as well as head and neck cancer, and associated metastases. Additional examples of cancer can be found in The Merck Manual of Diagnosis and Therapy, 19th Edition, § on Hematology and Oncology, published by Merck Sharp & Dohme Corp., 2011 (ISBN 978-0-911910-19-3); The Merck Manual of Diagnosis and Therapy, 20th Edition, § on Hematology and Oncology, published by Merck Sharp & Dohme Corp., 2018 (ISBN 978-0-911-91042-1) (2018 digital online edition at internet website of Merck Manuals); and SEER Program Coding and Staging Manual 2016, each of which are incorporated by reference in their entirety for all purposes.

In some embodiments, the cancer is a solid tumor. In some embodiments, the cancer is a breast, prostate, urinary bladder, skin, lung, ovary, sarcoma, or brain cancer. In some embodiments, the cancer is a liquid tumor such as, but not limited to a leukemia, including chronic leukemia, e.g., chronic myelocytic (granulocytic) leukemia and chronic lymphocytic leukemia, acute leukemia, e.g., acute lymphocytic leukemia, acute myelocytic leukemia, and myeloblastic, promyelocytic, myelomonocytic, monocytic and erythroleukemia, lymphoma, Waldenstrom's macroglobulinemia, Hodgkin's disease, non-Hodgkin's lymphoma, polycythemia vera, multiple myeloma, and heavy chain disease.

The therapeutic methods described herein may be used to treat a cancer expressing, e.g., CD19, CD22, CD123, CD33, B7-H3, HER2, IL13Rα2, or EphA2.

In some embodiments, the cancer is a cancer expressing HER2. Cancers expressing HER2 may include, but are not limited to, sarcomas such as angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, or synovial sarcoma; brain cancers such as glioblastoma; breast, prostate, lung, and colon cancers or epithelial cancers/carcinomas such as breast cancer, colon cancer, prostate cancer, head and neck cancer, skin cancer; cancers of the genitourinary tract such as ovarian cancer, endometrial cancer, cervical cancer and kidney cancer; lung cancer, gastric cancer, cancer of the small intestine, liver cancer, pancreatic cancer, gall bladder cancer, cancers of the bile duct, esophagus cancer, cancer of the salivary glands and cancer of the thyroid gland. In some embodiments, the cancer is a HER2-positive breast cancer. As a non-limiting example, the cancer is a HER-2-positive breast cancer.

In some embodiments, the cancer is a cancer expressing IL13Rα2. Cancers expressing IL13Rα2 may include, but are not limited to, brain cancers such as glioblastoma, colon cancer, renal cell carcinoma, pancreatic cancer, melanoma, head and neck cancer, mesothelioma, and ovarian cancer. In some embodiments, the cancer is an IL13Rα2-positive glioblastoma. As a non-limiting example, the cancer is an IL13Rα2-positive glioblastoma.

In some embodiments, the cancer is a cancer expressing EphA2. Cancers expressing EphA2 may include, but are not limited to, sarcomas such as rhabdomyosarcoma, osteosarcoma, and Ewings sarcoma; breast, prostate, urinary bladder, skin cancers including melanoma, lung cancer, liver cancer, ovarian cancer, stomach cancer, colorectal cancer, thyroid cancer, head and neck cancer, cervical cancer, pancreatic cancer, endometrial cancer, and brain cancers.

In some embodiments, the cancer is a cancer expressing B7-H3. Cancers expressing B7-H3 may include, but are not limited to, osteosarcoma, rhabdomyosarcoma, Ewing's sarcoma and other Ewing's sarcoma family of tumors, neuroblastoma, ganglioneuroblastoma, desmoplastic small round cell tumor, malignant peripheral nerve sheath tumor, synovial sarcoma, undifferentiated sarcoma, adrenocortical carcinoma, hepatoblastoma, Wilms tumor, rhabdoid tumor, high grade glioma (glioblastoma multiforme), medulloblastoma, astrocytoma, glioma, ependymoma, atypical teratoid rhabdoid tumor, meningioma, craniopharyngioma, primitive neuroectodermal tumor, diffuse intrinsic pontine glioma and other brain tumors, acute myeloid leukemia, multiple myeloma, lung cancer, mesothelioma, breast cancer, bladder cancer, gastric cancer, prostate cancer, colorectal cancer, endometrial cancer, cervical cancer, renal cancer, esophageal cancer, ovarian cancer, pancreatic cancer, hepatocellular carcinoma and other liver cancers, head and neck cancers, leiomyosarcoma, and melanoma.

In some embodiments, the therapeutic methods include genetically modifying the immune effector cell to express a chimeric antigen receptor (CAR) that is capable of binding specifically to an antigen. In some embodiments, the antigen is a tumor antigen. Non-limiting examples of tumor antigens include human epidermal growth factor receptor 2 (HER2), interleukin-13 receptor subunit alpha-2 (IL-13Ra2), ephrin type-A receptor 2 (EphA2), A kinase anchor protein 4 (AKAP-4), adrenoceptor beta 3 (ADRB3), anaplastic lymphoma kinase (ALK), immunoglobulin lambda-like polypeptide 1 (IGLL1), androgen receptor, angiopoietin-binding cell surface receptor 2 (Tie 2), B7-H3 (CD276), bone marrow stromal cell antigen 2 (BST2), carbonic anhydrase IX (CAIX), CCCTC-binding factor (Zinc Finger Protein)-like (BORIS), CD171, CD179a, CD24, CD300 molecule-like family member f (CD300LF), CD38, CD44v6, CD72, CD79a, CD79b, CD97, chromosome X open reading frame 61 (CXORF61), claudin 6 (CLDN6), CS-1 (CD2 subset 1, CRACC, SLAMF7, CD319, or 19A24), C-type lectin domain family 12 member A (CLEC12A), C-type lectin-like molecule-1 (CLL-1), Cyclin B 1, Cytochrome P450 1B 1 (CYP1B 1), EGF-like module-containing mucin-like hormone receptor-like 2 (EMR2), epidermal growth factor receptor (EGFR), ERG (transmembrane protease, serine 2 (TMPRSS2) ETS fusion gene), ETS translocation-variant gene 6, located on chromosome 12p (ETV6-AML), Fc fragment of IgA receptor (FCAR), Fc receptor-like 5 (FCRL5), Fms-like tyrosine kinase 3 (FLT3), Folate receptor beta, Fos-related antigen 1, Fucosyl GM1, G protein-coupled receptor 20 (GPR20), G protein-coupled receptor class C group 5, member D (GPRC5D), ganglioside GD3, ganglioside GM3, glycoceramide (GloboH), Glypican-3 (GPC3), Hepatitis A virus cellular receptor 1 (HAVCR1), hexasaccharide portion of globoH, high molecular weight-melanoma-associated antigen (HMWMAA), human Telomerase reverse transcriptase (hTERT), interleukin 11 receptor alpha (IL-1 IRa), KIT (CD 117), leukocyte-associated immunoglobulin-like receptor 1 (LAIR1), leukocyte immunoglobulin-like receptor subfamily A member 2 (LILRA2), Lewis(Y) antigen, lymphocyte antigen 6 complex, locus K 9 (LY6K), lymphocyte antigen 75 (LY75), lymphocyte-specific protein tyrosine kinase (LCK), mammary gland differentiation antigen (NY-BR-1), melanoma cancer testis antigen-1 (MAD-CT-1), melanoma cancer testis antigen-2 (MAD-CT-2), melanoma inhibitor of apoptosis (ML-IAP), mucin 1, cell surface associated (MUC1), N-acetyl glucosaminyl-transferase V (NA17), neural cell adhesion molecule (NCAM), o-acetyl-GD2 ganglioside (OAcGD2), olfactory receptor 51E2 (OR51E2), p53 mutant, paired box protein Pax-3 (PAX3), paired box protein Pax-5 (PAX5), pannexin 3 (PANX3), placenta-specific 1 (PLAC1), platelet-derived growth factor receptor beta (PDGFR-beta), Polysialic acid, proacrosin binding protein sp32 (OY-TES 1), prostate stem cell antigen (PSCA), Protease Serine 21 (PRSS21), Proteasome (Prosome, Macropain) Subunit, Beta Type, 9 (LMP2), Ras Homolog Family Member C (RhoC), sarcoma translocation breakpoints, sialyl Lewis adhesion molecule (sLe), sperm protein 17 (SPA17), squamous cell carcinoma antigen recognized by T cells 3 (SART3), stage-specific embryonic antigen-4 (SSEA-4), synovial sarcoma, X breakpoint 2 (SSX2), TCR gamma alternate reading frame protein (TARP), TGS5, thyroid stimulating hormone receptor (TSHR), Tn antigen (Tn Ag), tumor endothelial marker 1 (TEM1/CD248), tumor endothelial marker 7-related (TEM7R), uroplakin 2 (UPK2), vascular endothelial growth factor receptor 2 (VEGFR2), v-myc avian myelocytomatosis viral oncogene neuroblastoma derived homolog (MYCN), Wilms tumor protein (WTi), and X Antigen Family, Member 1A (XAGE1), or a fragment or variant thereof, and any other tumor antigens that are described herein.

In cases where the immune effector cell is isolated from a donor, the method may further include a method to prevent graft vs host disease (GVHD) and the immune effector cell rejection.

In some embodiments, the therapeutic methods include isolating an immune effector cell from the subject or a donor; modifying a DNMT3A gene or gene product in the immune effector cell such that the DNMT3A-mediated de novo DNA methylation of the cell genome is inhibited; enhancing the IL10 signaling pathway in the immune effector cell by either subjecting the immune effector cell to an exogenous IL10 or genetically modifying the immune effector cell to express IL10 using; and, introducing the modified immune effector cell into the subject. In some embodiments, the subjecting the immune effector cell to an exogenous IL10 is carried out ex vivo. In some embodiments, the subjecting the immune effector cell to an exogenous IL10 is carried out in vivo. In some embodiments, the exogenous IL10 is delivered to the immune effector cell in vivo in a carrier such as, but not limited to, an oncolytic virus or nanoparticle.

In some embodiments of any of the therapeutic methods described above, the method may further comprise the immune effector cell to express a chimeric antigen receptor (CAR) capable of binding specifically to an antigen. In some embodiments, the therapeutic methods include genetically modifying the immune effector cell to express a T cell receptor (TCR) that is capable of binding specifically to an antigen.

In some embodiments, the subject is human.

In some embodiments of any of the therapeutic methods described above, the composition is administered in a therapeutically effective amount. The dosages of the composition administered in the methods of the invention will vary widely, depending upon the subject's physical parameters, the frequency of administration, the manner of administration, the clearance rate, and the like. The initial dose may be larger, and might be followed by smaller maintenance doses. The dose may be administered as infrequently as weekly or biweekly, or fractionated into smaller doses and administered daily, semi-weekly, etc., to maintain an effective dosage level. It is contemplated that a variety of doses will be effective to achieve in vivo persistence of immune effector cells. It is also contemplated that a variety of doses will be effective to improve in vivo effector function of immune effector cells.

In some embodiments, composition comprising the immune effector cells manufactured by the methods described herein may be administered at a dosage of 10² to 10¹⁰ cells/kg body weight, 10⁵ to 10⁹ cells/kg body weight, 10⁵ to 10⁸ cells/kg body weight, 10⁵ to 10⁷ cells/kg body weight, 10⁷ to 10⁹ cells/kg body weight, or 10⁷ to 10⁸ cells/kg body weight, including all integer values within those ranges. The number of immune effector cells will depend on the therapeutic use for which the composition is intended for.

Modified immune effector cells may be administered multiple times at dosages listed above. The immune effector cells may be allogeneic, syngeneic, xenogeneic, or autologous to the patient undergoing therapy.

The compositions and methods described in the present disclosure may be utilized in conjunction with other types of therapy for cancer, such as chemotherapy, surgery, radiation, gene therapy, and so forth.

It is also contemplated that when used to treat various diseases/disorders, the compositions and methods of the present disclosure can be utilized with other therapeutic methods/agents suitable for the same or similar diseases/disorders. Such other therapeutic methods/agents can be co-administered (simultaneously or sequentially) to generate additive or synergistic effects. Suitable therapeutically effective dosages for each agent may be lowered due to the additive action or synergy.

In some embodiments of any of the above therapeutic methods, the method further comprises administering to the subject one or more additional compounds selected from the group consisting of immuno-suppressives, biologicals, probiotics, prebiotics, and cytokines (e.g., IFN or IL-2).

As a non-limiting example, the invention can be combined with other therapies that block inflammation (e.g., via blockage of IL1, INFα/β, IL6, TNF, IL23, etc.).

The methods and compositions of the invention can be combined with other immunomodulatory treatments such as, e.g., therapeutic vaccines (including but not limited to GVAX, DC-based vaccines, etc.), checkpoint inhibitors (including but not limited to agents that block CTLA4, PD1, LAG3, TIM3, etc.) or activators (including but not limited to agents that enhance 4-1BB, OX40, etc.). The methods of the invention can be also combined with other treatments that possess the ability to modulate NKT function or stability, including but not limited to CD1d, CD1d-fusion proteins, CD1d dimers or larger polymers of CD1d either unloaded or loaded with antigens, CD1d-chimeric antigen receptors (CD1d-CAR), or any other of the five known CD1 isomers existing in humans (CD1a, CD1b, CD1c, CD1e). The methods of the invention can also be combined with other treatments such as midostaurin, enasidenib, or a combination thereof.

Therapeutic methods of the invention can be combined with additional immunotherapies and therapies. For example, when used for treating cancer, the compositions of the invention can be used in combination with conventional cancer therapies, such as, e.g., surgery, radiotherapy, chemotherapy or combinations thereof, depending on type of the tumor, patient condition, other health issues, and a variety of factors. In certain aspects, other therapeutic agents useful for combination cancer therapy with the inhibitors of the invention include anti-angiogenic agents. Many anti-angiogenic agents have been identified and are known in the art, including, e.g., TNP-470, platelet factor 4, thrombospondin-1, tissue inhibitors of metalloproteases (TIMP1 and TIMP2), prolactin (16-Kd fragment), angiostatin (38-Kd fragment of plasminogen), endostatin, bFGF soluble receptor, transforming growth factor beta, interferon alpha, soluble KDR and FLT-1 receptors, placental proliferin-related protein, as well as those listed by Carmeliet and Jain (2000). In one embodiment, the immune effector cells of the invention can be used in combination with a VEGF antagonist or a VEGF receptor antagonist such as anti-VEGF antibodies, VEGF variants, soluble VEGF receptor fragments, aptamers capable of blocking VEGF or VEGFR, neutralizing anti-VEGFR antibodies, inhibitors of VEGFR tyrosine kinases and any combinations thereof (e.g., anti-hVEGF antibody A4.6.1, bevacizumab or ranibizumab).

Non-limiting examples of chemotherapeutic compounds which can be used in combination treatments of the present invention include, for example, aminoglutethimide, amsacrine, anastrozole, asparaginase, azacitidine, bcg, bicalutamide, bleomycin, buserelin, busulfan, campothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, decitabine, dienestrol, diethylstilbestrol, docetaxel, doxorubicin, epirubicin, estradiol, estramnustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, ironotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic compounds may be categorized by their mechanism of action into, for example, following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethyhnelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (e.g., TNP-470, genistein, bevacizumab) and growth factor inhibitors (e.g., fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers and caspase activators; and chromatin disruptors.

In various embodiments of the methods described herein, the subject is a human. The subject may be a juvenile or an adult, of any age or sex.

In accordance with the present invention there may be numerous tools and techniques within the skill of the art, such as those commonly used in molecular biology, pharmacology, and microbiology. Such tools and techniques are described in detail in e.g., Sambrook et al. (2001) Molecular Cloning: A Laboratory Manual. 3rd ed. Cold Spring Harbor Laboratory Press: Cold Spring Harbor, New York; Ausubel et al. eds. (2005) Current Protocols in Molecular Biology. John Wiley and Sons, Inc.: Hoboken, NJ; Bonifacino et al. eds. (2005) Current Protocols in Cell Biology. John Wiley and Sons, Inc.: Hoboken, NJ; Coligan et al. eds. (2005) Current Protocols in Immunology, John Wiley and Sons, Inc.: Hoboken, NJ; Coico et al. eds. (2005) Current Protocols in Microbiology, John Wiley and Sons, Inc.: Hoboken, NJ; Coligan et al. eds. (2005) Current Protocols in Protein Science, John Wiley and Sons, Inc.: Hoboken, NJ; and Enna et al. eds. (2005) Current Protocols in Pharmacology, John Wiley and Sons, Inc.: Hoboken, NJ.

### EXAMPLES

The following examples are provided to further describe some of the embodiments disclosed herein. The examples are intended to illustrate, not to limit, the disclosed embodiments.

### Example 1. IL10 Promotes DNMT3A KO CAR T-Cell Survival with Chronic Antigen Exposure

DNMT3A-mediated regulation of human T-cell developmental potential is coupled to the epigenetic repression of stem-associated genes. This Example investigated the signaling network supporting acquisition of the exhaustion program. From in vitro chronic stimulation studies, IL10 was identified as the dominant differentially expressed cytokine produced by DNMT3A KO CAR T-cells. Briefly, DNMT3A KO and Ctrl HER2.ζ-, HER2.CD28 ζ-, or IL13Rα2.CD28 ζ-CAR T-cells were cultured with U373 cells and after 24 hours the concentration of Th1 (GM-CSF, IFNγ, TNFα, IL-2) and Th2 (IL4, IL5, IL6, IL10, IL13) cytokines was determined by multiplex analysis. Comparison of Ctrl and DNMT3A KO CAR T-cells after one round of stimulation revealed that all analyzed cytokines were secreted at similar levels, except IL10, which was significantly higher in samples from DNMT3A KO CAR T-cells. The same analysis was performed after the 4th stimulation, further highlighting IL10 as the only differentially expressed cytokine, and suggesting a role for IL10 signaling in CAR T-cell survival. The cytolytic capacity of control and DNMT3A KO CAR T-cells was also determined at the 1st and 4th stimulation. DNMT3A KO allowed CAR T-cells to retain the ability to kill target cells at later stimulations compared to Ctrl CAR T-cells.

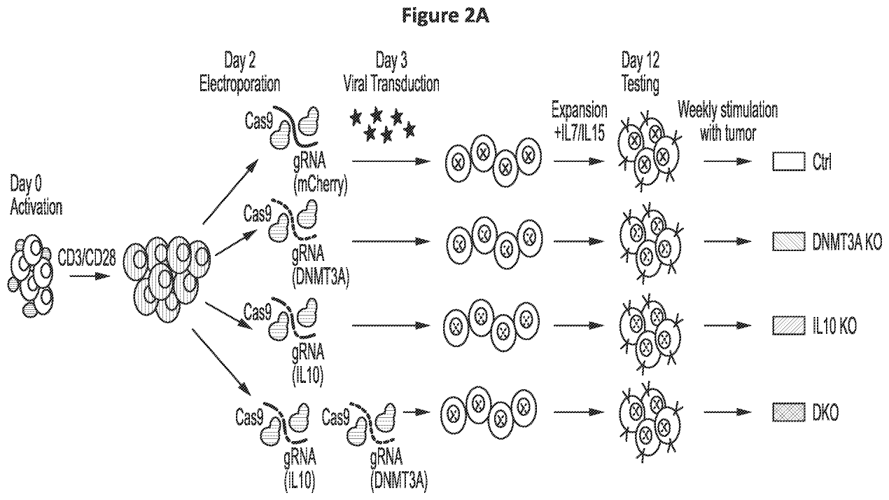

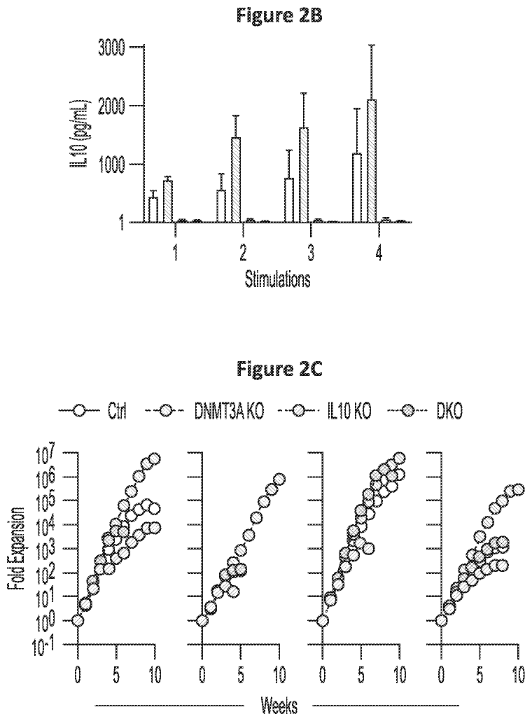

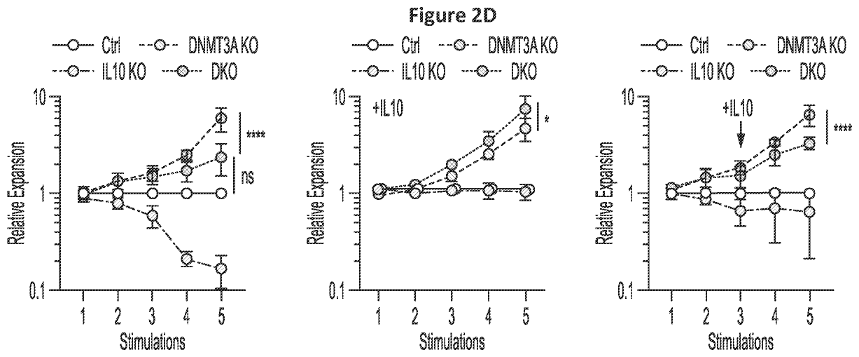

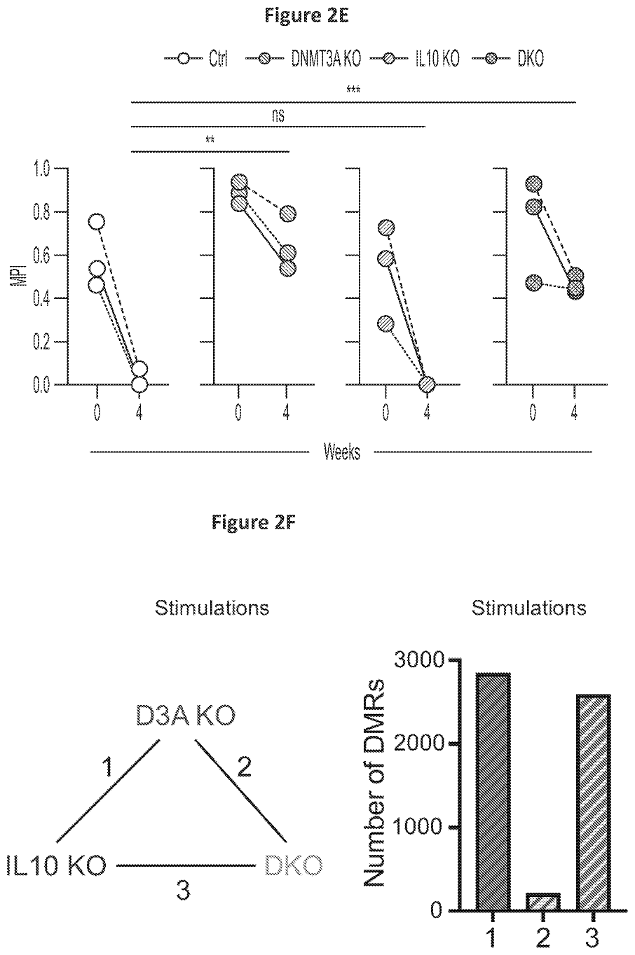

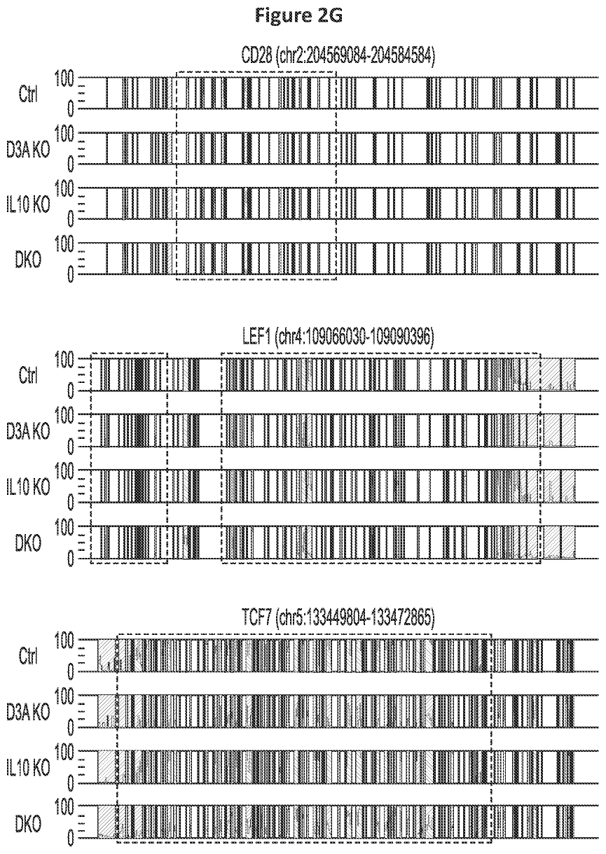

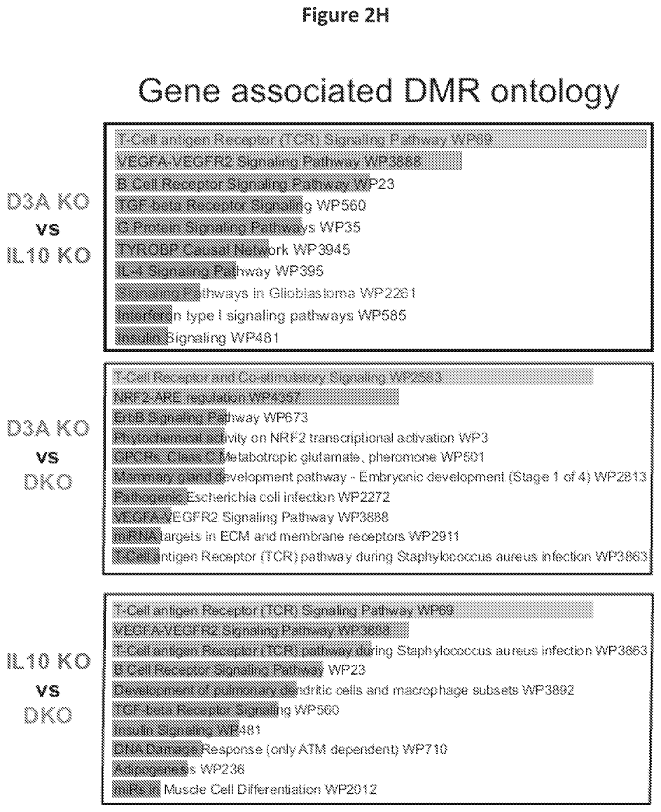

To investigate if IL10 played a causal role, an IL10 gene expression signature based on published gene expression profiles of human T-cells cultured with or without IL10 was first established. Cross referencing this signature with the DNMT3A gene target list, a statistically significant enrichment of genes that are coupled to IL10 signaling among the DNMT3A target genes was found (FIG. 1A). To determine the role of IL10 in the context of DNMT3A KO, HER2.ζ-CAR T-cells in which either IL10, DNMT3A, or both (Double knockout, DKO) were deleted by CRISPR/Cas9 gene editing were generated (FIG. 2A and FIGS. 1B-F). Ctrl, DNMT3A KO, IL10 KO and DKO CAR T-cells were stimulated weekly with U373 tumor cells in the presence of IL15 with or without exogenous IL10 (FIG. 2A), and the lack of IL10 production by IL10 KO and DKO CAR T-cells was confirmed by ELISA (FIG. 2B). While DNMT3A KO CAR T-cells had an expected significant proliferative advantage in comparison to Ctrl CAR T-cells, this sustained proliferative capacity was diminished in DKO CAR T-cells (FIGS. 2C-2D). In the presence of IL10, this difference was reversed, confirming that IL10 supports proliferation of CAR T-cells.

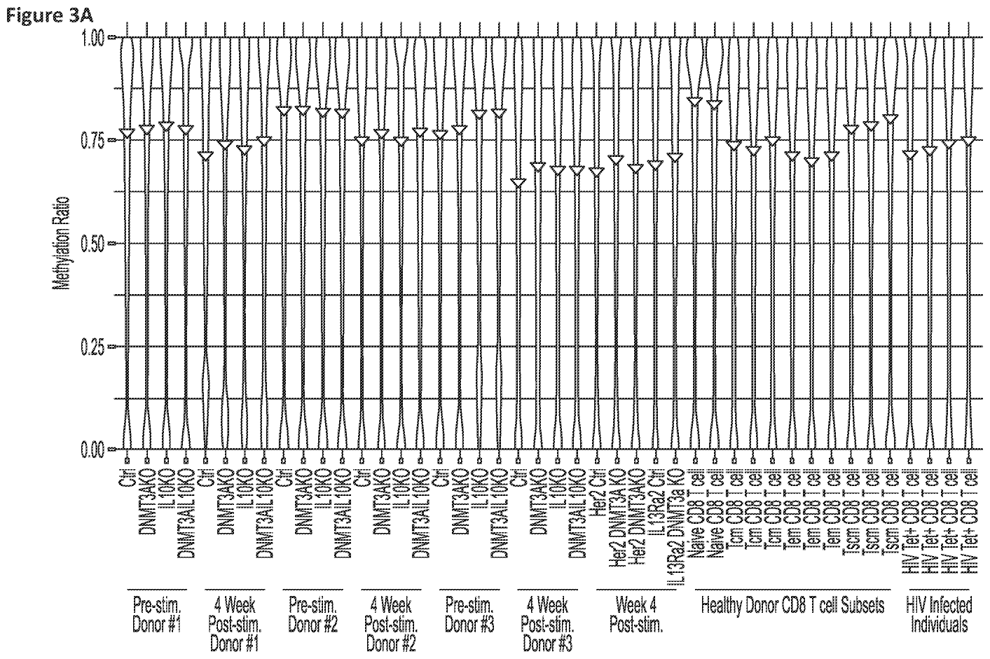

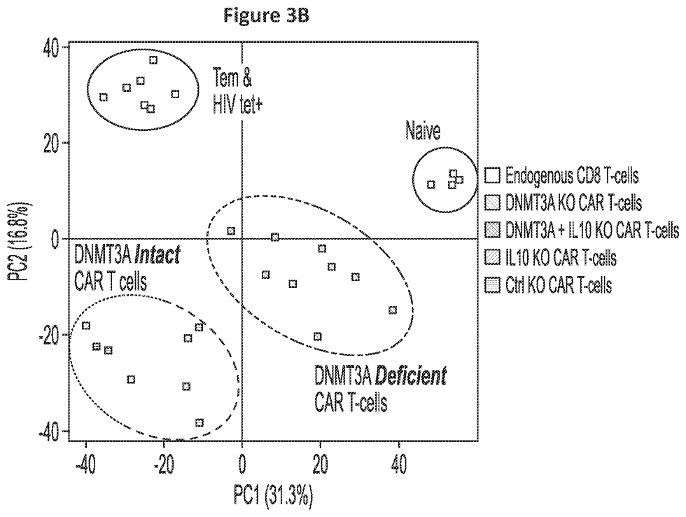

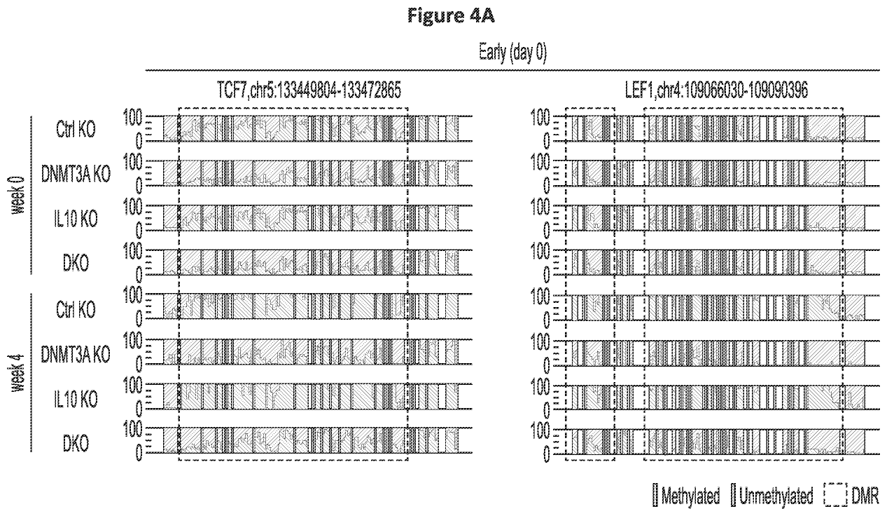

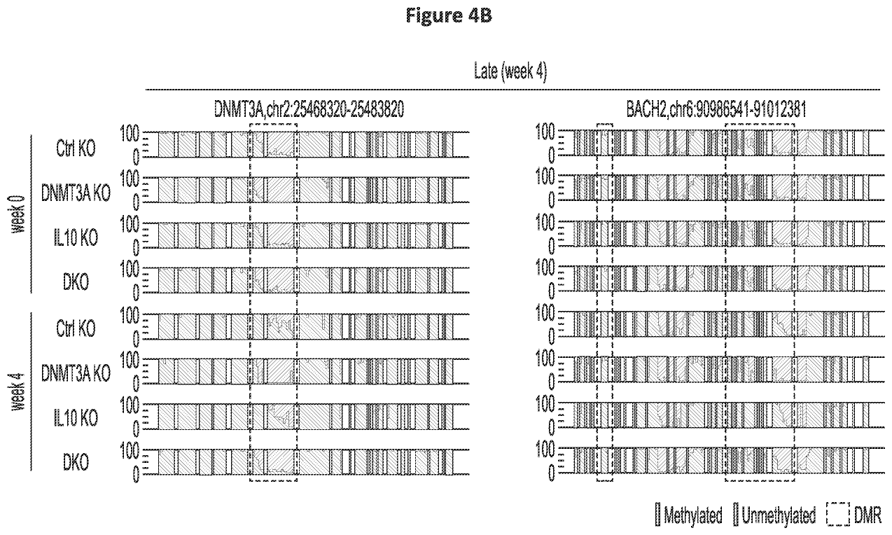

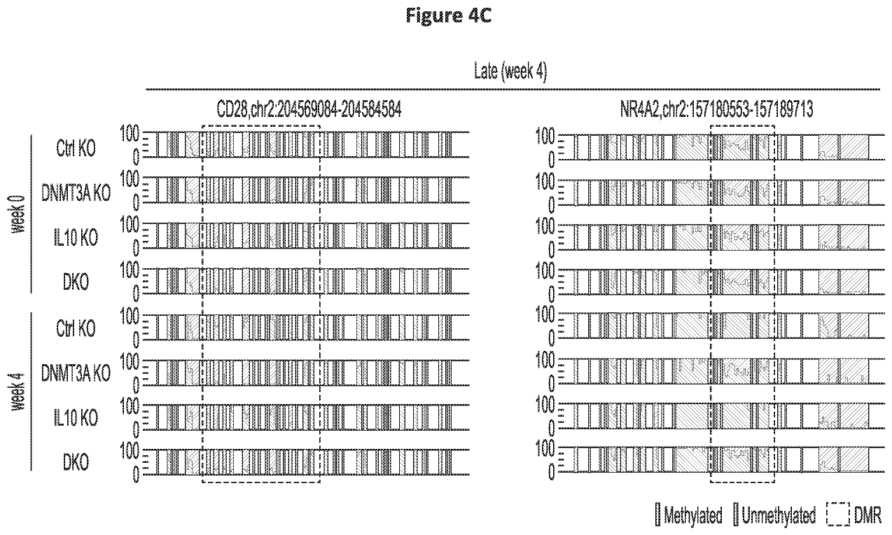

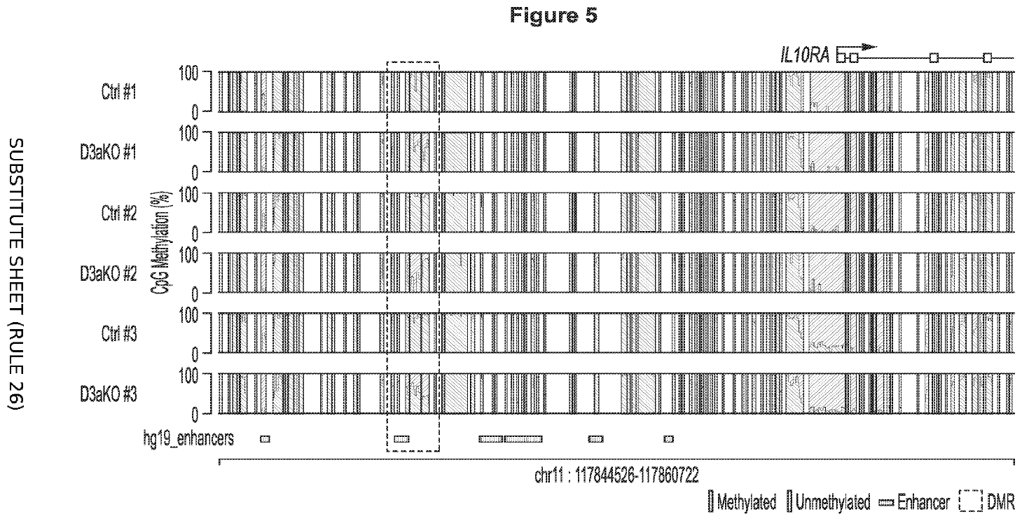

Whole-genome methylation profiling was then performed to assess the epigenetic state of all four CAR T-cell populations prior to the 1^{st} and after the 4^{th} stimulation with tumor cells (FIGS. 3A-3B). Principle component analysis (PCA) of whole-genome methylation profiles revealed that DNMT3A-deficient (DNMT3A KO, DKO) and DNMT3A-intact (Crtl, IL10 KO) CAR T-cell populations formed distinct clusters (FIG. 3B). Consistent with previous findings, DNMT3A-mediated methylation of the stem-associated genes, TCF7 and LEF1, occurred early in contrast to acquisition of methylation at other loci, including CD28 (FIG. 4). Likewise, DNMT3A KO CAR T-cells retained a high methylation-based multipotency index (MPI) in contrast to Ctrl CAR T-cells (FIG. 2E). Deletion of IL10 in DNMT3A KO CAR T-cells reduced the MPI (FIG. 2E). Consistent with the PCA, the differentially methylated regions (DMRs) were highly conserved between DNMT3A KO versus IL10 KO and DKO versus IL10 KO CAR T-cells (i.e., DNMT3A-deficient CAR T-cells have a similar methylation profile) (FIG. 2F). Shared DMRs included aforementioned genes regulating T-cell stemness such as TCF7, LEF1, and CD28 (FIG. 2G and FIGS. 3A-3B). However, GO analysis performed on DNMT3A KO versus DKO DMR lists also highlighted differences, including co-stimulation (FIG. 2H). Taken together with the observation that exhaustion-associated CD28 promotor methylation occurred in Ctrl but not DNMT3A KO CAR T-cells, these data support a model in which IL10 signaling serves as a negative feedback loop to limit CD28 co-stimulation in the setting of chronic antigen exposure. Lastly, consistent with the conclusion that DNMT3A deficient CAR T cells retain an ability to respond to IL10 signaling, methylation profiling among the week 4 control (Ctrl), DNMT3A KO, IL10 KO, and DNMT3A+IL10 double KO (DKO) CAR T-cells revealed an enhancer upstream of the IL10RA promoter remained unmethylated in the KO CAR T cells relative to the control edited CD8+ CAR T cells (FIG. 5).

Notably, the data herein have identified CD28, and the downstream T-cell stemness (TCF7 and LEF1) pathways, as targets for epigenetic silencing during the progressive development of T-cell exhaustion. Recent efforts to identify the population of T-cells that contribute to therapeutic efficacy in models of immunotherapy have similarly identified CD28+ T-cells as the primary source for antitumor responses (55, 56). These results taken together with the data presented herein, as well as studies reporting a critical role for IL10 in suppressing T-cell effector responses (57), suggest that the increase in IL10 signaling that observe in exhaustion-resistant DNMT3A KO CAR T-cells serves to limit the cells from becoming over-stimulated. These data highlighted DNMT3A as a central mediator of T-cell exhaustion and suggested that this epigenetic process may serve as a universal mechanism to restrict mammalian T-cell fate potential.

The increased functionality of the exhaustion-resistant DNMT3A KO CAR T-cells was coupled to an upregulation of IL10 and genome-wide DNA methylation profiling defined an atlas of genes targeted for epigenetic silencing.

Below are the methods used in the Example described above.

Cell lines: U373 glioma and 293T (human embryonic kidney) cells were purchased from the American Type Culture Collection (ATCC) (Manassas, VA). LM7 osteosarcoma cells were kindly provided by Dr. Eugenie Kleinerman (MD Anderson Cancer Center, Houston, TX) in 2011. U373 cells were maintained in RPMI 1640 supplemented with 10% fetal bovine serum and 2 mmol/L GlutaMAX while LM7 and 293T were maintained in Dulbecco's Modified Eagle Medium (DMEM) with the same supplements. The production of U373 and LM7 cells expressing enhanced green fluorescent protein and firefly luciferase (eGFP.ffLuc) has been previously described (34, 63). Cell lines were routinely validated using the ATCC STR Profiling Cell Authentication Service and tested for mycoplasma on a regular basis.

Production of retroviral vectors: The generation of retroviral vectors encoding HER2-, IL13Rα2-, or EphA2-CARs with a CD28 transmembrane domain and CD28(signaling domain, or IL-15 was previously described (35-37). The retroviral vector encoding the first generation HER2.ζ-CAR was created by subcloning the HER2-specific scFv, FRP5 (35, 64), into a retroviral vector encoding an expression cassette with a CD28 transmembrane and (signaling domain (65). To create transgenic DNMT3A, DNMT3A ORF (NM_175629.2, GenScript, Piscataway NJ) was cloned into a retroviral pSFG vector downstream of CD20 and a 2A sequence with a FLAG-tag added to the C-terminus using NEBuilder (New England BioLabs, Ipswich, MA). In order to make the sequence resistant to cleavage by Cas9, two silent mutations, one in the sgRNA recognition sequence and one in the PAM, were incorporated using QuikChange Lightning Mutagenesis kit (Agilent Technologies, Santa Clara, CA). RD114-pseudotyped retroviral particles were generated as previously described (63) by transient transfection of 293T-cells using GeneJuice Transfection Reagent (EMD Millipore, Burlington, MA). Viral supernatants were collected 48 hours post-transfection for the same day T-cell transduction or snap-frozen, and stored at −80° C. until use.

Generation of DNMT3A knockout CAR T-cells: Peripheral blood mononuclear cells (PBMCs) were isolated from consented healthy donors via density gradient separation using Lymphoprep (StemCell Technologies, Vancouver, BC). Cells were then plated in 24-well non-tissue culture-treated plates pre-coated with 250 ng each of anti-CD3 and anti-CD28 monoclonal antibodies (Miltenyi Biotec, Bergisch Gladbach, Germany). Culture medium for initial stimulation was RPMI 1640 supplemented with 10% fetal bovine serum and 2 mmol/L GlutaMAX (Thermo Fisher, Waltham, MA). IL-7 and IL-15 were added at 10 ng/mL and 5 ng/mL, respectively, 24 hours later. The following day, cells were electroporated with S. pyogenes Cas9-single guide RNA RNP complexes targeting DNMT3A, IL10 or mCherry (Control; Ctrl) followed 24 hours later by transduction on RetroNectin (Takara Bio, Mountain View, CA)-coated plates. RNPs were pre-complexed at a sgRNA:Cas9 ratio of 4.5:1, prepared by adding 3 μL 60 μM sgRNA (Synthego, Menlo Park, CA) to 1 μL 40 μM Cas 9 (Macro Lab, University of California, Berkeley) and frozen for later use. For single gene knockout, 6×10⁵ T-cells were resuspended in 20 μL R buffer and added to 4 μL RNP. For double gene knockout, 6×10⁵ T-cells were resuspended in 16 μL R buffer and added to 4 μL RNP1 (DNMT3A) and 4 μL RNP2 (IL10). 10 μL cells+ RNP were electroporated with 3 pulses of 10 milliseconds at 1600V using the Neon Transfection SystemThermo Fisher, Waltham, MA). Two 10 μL electroporation reactions were pooled in one well of a 48-well tissue-culture treated plate containing RPMI 1640 supplemented with 20% FBS, Glutamax, 10 ng/mL IL-7, and 5 ng/mL IL-15 for 72 hours. Following recovery, the media was switched to RPMI 1640 containing 10% FBS and GlutaMAX. The cells were then expanded for 10-12 days with IL-7 and IL-15 added every 2-3 days at the same concentrations indicated above.

Transgenic DNMT3A (tD) CAR T-cells: For CAR T-cells expressing transgenic DNMT3A, cells were prepared as described previously, the lone modification being that T-cells were electroporated in the morning, transduced with the vector encoding tD in the afternoon, and transduced again the following morning with the vector encoding the HER2.ζCAR.

Guide RNA design and validation: sgRNAs were designed to target unique sites within the genome with at least 3 bp of mismatch between the target site and any other site in the genome whenever possible and common SNPs were avoided. sgRNAs targeting the DNMT3A catalytic domain located in exon 19 were designed to disrupt DNMT3A function (32). An additional sgRNA (guide 3) that was previously validated and published was used to mitigate the risk of any off-target mediated functional effects (32). As an additional control, sgRNAs were designed to a sequence in mCherry that does not occur in the human genome with up to 3 bp of mismatch. To assess activity, DNMT3A or IL10 sgRNAs were nucleofected as RNPs (100 μM sgRNA and 50 μM Cas9 protein using solution P3 and program FF-120) into K562, genomic DNA from transfected cells was harvested three days post-nucleofection, and non-homologous end joining (NHEJ) rates were determined using targeted next-generation sequencing (NGS) followed by analysis with CRIS.py (66). sgRNAs were then transfected into T-cells as described in previous section and blotted by Western to further validate activity (loss of protein). IL10 protospacer: 5′ GTTGTTAAAGGAGTCCTTGCNGG 3′, DNMT3A protospacer: g2-5′ CCTGCATGATGCGCGGCCCANGG 3′ (SEQ ID NO: 63), g3-5′ GCATGATGCGCGGCCCAAGGNGG 3′(SEQ ID NO: 68); mCherry protospacer: g17-5′ CAAGTAGTCGGGGATGTCGGNGG 3′(SEQ ID NO: 64), g19-5′ AGTAGTCGGGATGTCGGCGNGG 3′ (SEQ ID NO: 65). Primer sequences used to amplify the region of interest for NGS indicated below:

DNMT3A: SP230.DS.F-
(SEQ ID NO: 71)
5′ TCCCGATGACCCTGTCTTCCCGTGC 3′;
SP230.DS.R-
(SEQ ID NO: 72)
5′ AGGTAGAAGCCATTAGTGAGCTGGC 3′.
IL10:
CAGE652.IL10.F-
(SEQ ID NO: 73)
5′ GGTCCCATCACTGTTGAATCCTCTGT 3′
CAGE652.IL10.R-
(SEQ ID NO: 74)
5′ ACCCTCTGGCTGCTGGATGTGCTGA 3′.

Western blot analysis: Knockout efficiency was determined by Western Blot. Ctrl and DNMT3A KO CAR T-cells were washed with PBS and lysed with RIPA buffer Cell Signaling Technologies, Danvers, MA) the day of their first use in experiments. Protein quantification was performed using a BCA assay (Thermo Fisher Scientific, Waltham, MA), and 30 pg total protein was loaded on a 12% SDS polyacrylamide gel and then transferred to nitrocellulose membranes (BioRad, Hercules, CA). Membranes were blocked with 5% milk in TBS+0.1% Tween-20 (Sigma Aldrich, St Louis, MO) for 1 hour at room temperature. The membranes were then probed with a monoclonal rabbit human DNMT3A antibody (Cell Signaling Technologies, Danvers, MA) or polyclonal GAPDH antibody (Santa Cruz Biotechnologies, Dallas, TX) overnight at 4° C. Membranes were then washed and probed with a secondary anti-Rabbit IgG-HRP (Jackson ImmunoResearch, West Grove, PA) for 1 hour at room temperature. Protein expression was visualized using Femto Enhanced Chemiluminescent Substrate (Thermo Fisher Scientific, Waltham, MA), and images were acquired and analyzed using a Li-cor Odyssey instrument and software (Li-Cor Biosciences, Lincoln, NE).

Various in vitro functional assays were performed according to methods as described below.

Repeated stimulation assay: Ctrl and DNMT3A KO CAR T-cells were co-cultured with U373 or LM7 cells at an effector to target ratio of 2:1 in the presence of IL-15 (5 ng/mL). Seven days later, T-cells were counted and replated with fresh tumor cells at the same 2:1 ratio in the presence of IL15 (5 ng/mL). The T-cells continued to be counted and stimulated with fresh tumor cells on a weekly basis until the T-cells stopped killing tumor cells. Co-cultures with Ctrl-, DNMT3A KO-, IL10 KO-, and DKO-CAR T-cells were also performed in the presence or absence of IL10 (5 ng/mL; Peprotech, Rocky Hill, NJ).

Cytokine production: At each stimulation of the repeated stimulation assay, culture supernatant was collected 24 hours after plating T-cells with tumor cells for analysis of cytokine production. Cytokine production was assessed by a 14-plex human cytokine quantification kit (Millipore Sigma, Burlington, MA) with analysis performed using a Luminex FlexMap 3D instrument and software (Luminex Corporation, Austin, TX). An IL10 ELISA (Peprotech, Rocky Hill, NJ) was used to confirm successful knockout of IL10.

Cytotoxicity assay: A CellTiter96@ AQueous One Solution Cell Proliferation Assay (Promega, Madison, WI) was utilized to assess CAR T-cell cytotoxicity as previously described (34). Briefly, tumor cells were incubated with varying amounts of CAR T-cells to assess cytotoxicity at a range of E:T ratios. 24 hours later, the media and T-cells were removed, and the remaining tumor cells were quantified with MTS reagent. Media only and tumor only served as controls to assess percent cytotoxicity. MTS assays were performed using unstimulated CAR T-cells (1^{st} stimulation) and CAR T-cells that had previously been exposed to tumor cells three times (4^{th} stimulation).

CAR T-cell antigen dependence assay: Survival of serially-stimulated DNMT3A KO CAR T-cells in the absence of tumor was assessed by positive selection of CD3+co-culture cells (Miltenyi Biotec, Bergisch Gladbach, Germany) followed by 7 day culture in the absence of tumor cells+/−IL-15. Live and dead cells were enumerated by flow cytometry using an eFluor 520 live/dead stain (Thermo Fisher Scientific, Waltham, MA) and annexin V APC (BD Biosciences, Franklin Lakes, NJ).

Flow cytometry and FACS analyses were performed according to methods as described below.

Immunophenotyping: Samples were phenotyped for cell surface antigens prior to and post-co-culture. Live cell discrimination was performed with an eFluor 506 fixable viability dye. The following antibodies were used in phenotypic analysis: CD45RO APC/Cy7 (Biolegend clone UCHL1), CCR7 FITC (Biolegend clone G043H7), PD1 BV421 (Biolegend clone EH12.2H7), Tim3 PeCy7 (Biolegend clone F38-2EZ), CD8 BV785 (Biolegend clone RPA-T8), Ghost Dye Violet 510 (Tonbo 13-0870-T100), TOX APC (Tonbo clone TXRX1O). TCF7 PE (Cell Signaling Technology 144565). Isotype-matched controls were run for each experiment. Surface staining was performed prior to fixation, permeabilization, and intracellular staining using the eBioscience Transcription Factor Staining Buffer Set (Thermo Fisher Scientific, Waltham, MA). Samples were acquired on a BD FACS Lyric, and list mode files were analyzed using FlowJo ver 10.5.3 (BD Biosciences, Franklin Lakes, NJ).

FACS sorting for whole-genome bisulfite sequencing (WGBS): WGBS was performed on CAR T-cells that were taken directly from culture or had been previously stimulated with fresh U373 tumor cells four times, with the stimulations separated by one week or one day, as indicated in the text. WGBS was also performed on T-cells extracted from i.p. LM7-bearing mice by intraperitoneal wash 7 days after T-cell injection. Ctrl KO-, DNMT3A KO-, and, for some conditions, IL10 KO- and DKO-CAR T-cells were FACS purified based on live CD8+ phenotyping using a BD FACS Aria II (BD Biosciences, Franklin Lakes, NJ). DNA was extracted and bisulfite converted (Zymo EZ DNA methylation-direct kit, Irvine, CA) which converted all unmethylated cytosines to uracils while protecting the methylated cytosines from deamination. Bisulfite-modified DNA libraries were sequenced using Illumina HiSeq and sequencing data were mapped to the HG19 human genome. Differences in CpG methylation across the genome were analyzed using IGV software.

T-cell subset isolation: For experiments using purified cell populations, PBMCs from fresh blood from healthy consented donors were isolated by centrifugation in BD Vacutainer CPT tubes (BD Biosciences, Franklin Lakes, NJ) and enriched prior to sorting. Following CD8 enrichment, naive and memory CD8 T-cell compartments were FACS purified. Cells were stained in sterile PBS containing 4% FBS with the same antibodies described above. Individual cell populations were sorted using a BD FACS Aria II (BD Biosciences, Franklin Lakes, NJ). Naive CD8 T-cells were defined as live CD8⁺, CCR7⁺, and CD45RO-cells. Of note, the naive compartment included Tscm phenotyped (CD95⁺) cells. The memory CD8 T-cell compartment was sorted based on live CD8⁺, CD45RO⁺ expression. The memory compartment included Tem (CCR7-) and Tcm (CCR7⁺) CD8 T-cells. Sorted cells were checked for purity and determined to be at least 95% of the desired phenotype.

Transduction efficiencies: Efficiency of retroviral transduction was determined by flow cytometry using the following antibodies: IL13Rα2-CAR: recombinant IL13Rα2-Fc fusion protein (R&D systems, Minneapolis, MN) followed by anti-human IgG Fc PE (Southern Biotech, Birmingham, AL); HER2-CAR: anti-mouse Fab Alexa-647 (Jackson ImmunoResearch, West Grove, PA); EphA2-CAR: anti-human CD19 PE (Beckman Coulter, Brea, CA) or anti-human Fab Alexa-647(Jackson ImmunoResearch, West Grove, PA); IL15 vector: anti-human NGFR BV421 (BD Biosciences, Franklin Lakes, NJ).

Bisulfite sequencing methylation profiling and data analysis: Genome DNA was isolated from FACS purified T-cells and processed as previously described (67). Briefly, DNA was extracted from the sorted cells using a DNA-extraction kit (Qiagen) and then bisulfite treated using an EZ DNA methylation kit (Zymo Research), or an EZ DNA methylation-direct kit (Zymo Research). The bisulfite-modified DNA-sequencing library was generated using the EpiGnome™ kit (Epicentre) per the manufacturer's instructions and then sequenced using an Illumina Hiseq or Novaseq instrument. Sequencing data were aligned to the hg19 genome using BSMAP2.74 (68). Differentially methylated regions (DMRs) were identified using Bioconductor package DSS (69). Statistical test of differentially methylated locus (DML) were first performed using DML test function (smoothing=TRUE) in DSS, the results were then used to detect differentially methylated regions using CallDMR function in DSS, the β-value threshold for calling DMR is 0.01. The minimum length for defining a DMR was set to 50 bps and the minimum number of CpG sites for defining a DMR is 3. The GSEA analyses were performed using 10⁸ DNMT3A-target transcription factors as gene signature on gene expression data from GSE23321 (70). Ingenuity pathway analysis was performed using DNMT3A-targeted genes. Enrichr was used for gene ontology analysis.

The present disclosure establishes a human CD8+ T-cell DNA methylation based multipotency index using a machine learning approach (71). Briefly, to identify the methylation state of CpG sites associated with the T-cell multipotent potential, a supervised analysis was performed between the methylomes from two naive and four HIV-specific CD8 T-cells (methylation difference>=0.4 and FDR<=0.01). This analysis resulted in identification of 245 CpGs sites that were hypomethylated in naive CD8⁺ T-cells compared to HIV-specific CD8⁺ T-cells. This set of the CpGs was then used as an input to the one-class logistic regression to calculate the multipotency signature using just the HIV-specific and naive CD8+ T-cells (Training data sets) (72, 73). Once the signature was obtained, it was then applied to the naive CD8⁺ T-cells, Tscm, and Tem from healthy donors, and CAR T-cell methylomes (test data sets). The score was calculated as the dot product between the DNA methylation value and the signature. The score was subsequently converted to the [0, 1] range. Data sets with multipotency indices closer to 1 were more similar to naive cells.

CHANGE-seq: Genomic DNA from T-cells was isolated using Gentra Puregene Kit according to manufacturer's instructions. Purified genomic DNA was tagmented with a custom Tn5-transposome to an average length of 400 bp, followed by gap repair with Kapa HiFi HotStart Uracil+ DNA Polymerase (KAPA Biosystems) and Taq DNA ligase (NEB). Gap-repaired tagmented DNA was treated with USER enzyme (NEB) and T4 polynucleotide kinase (NEB). Intramolecular circularization of the DNA was performed with T4 DNA ligase (NEB) and residual linear DNA was degraded by a cocktail of exonucleases containing Plasmid-Safe ATP-dependent DNase (Lucigen), Lambda exonuclease (NEB) and Exonuclease I (NEB). In vitro cleavage reactions were performed with 125 ng of exonuclease-treated circularized DNA, 90 nM of SpCas9 protein (NEB), Cas9 nuclease buffer (NEB) and 270 nM of sgRNA, in a 50 μL volume. Cleaved products were A-tailed, ligated with a hairpin adaptor (NEB), treated with USER enzyme (NEB) and amplified by PCR with barcoded universal primers NEBNext Multiplex Oligos for Illumina (NEB), using Kapa HiFi Polymerase (KAPA Biosystems). Libraries were quantified by qPCR (KAPA Biosystems) and sequenced with 150 bp paired-end reads on an Illumina NextSeq instrument. CHANGE-seq data analyses were performed using open-source CIRCLE-seq analysis software (github.com/tsailabSJ/circleseq).

DNMT3A gene expression score: Transcript counts were obtained from Fraietta et al, Nat Med 2018 (62) “Supplemental Table 5b: Transcriptomic profiling of CAR-stimulated CTL019 infusion products” and filtered, normalized, and analyzed using the R packages ‘edgeR’ (74) and ‘limma’ (75). Principal components analysis was conducted on the entirety of the expression data, revealing a single Partial Responder data point that was an obvious outlier in comparison to the other samples (and therefore excluded from downstream analyses). 1,033 gene identifiers matched the 1,298 previously identified DNMT3A targets and were used to calculate a relative DNMT3A-target expression score; briefly, the log 2-expression of each target gene was scaled to its mean-centered variation across the samples of the dataset, and those normalized expression values were then summed for each sample. In subsequent analyses this score was also calculated using a limited gene set that either included only those differentially expressed genes (DGEs) between in vitro DNMT3A knockout and wildtype cells (as assayed by Affymetrix Clariom S Human microarray; Ctrl n=3, KO n=8) or the intersection between these DGEs and the previously identified DNMT3 Å targets. The nonparametric Kruskal Wallis test and Mann-Whitney U test were used to assess significant variation across the patient outcomes defined by the originating study, and plots were generated with ggplot2 (76).

### REFERENCES

1. Park J H, Geyer M B, Brentjens R J. CD19-targeted CAR T-cell therapeutics for hematologic malignancies: interpreting clinical outcomes to date. Blood. 2016; 127(26):3312-20. doi: 10.1182/blood-2016-02-629063. PubMed PMID: 27207800; PMCID: 4929923.
2. Scarfo I, Maus M V. Current approaches to increase CAR T cell potency in solid tumors: targeting the tumor microenvironment. J Immunother Cancer. 2017; 5:28. doi: 10.1186/s40425-017-0230-9. PubMed PMID: 28331617; PMCID: PMC5359946.
3. Leibman R S, Richardson M W, Ellebrecht C T, Maldini C R, Glover J A, Secreto A J, Kulikovskaya I, Lacey S F, Akkina S R, Yi Y J, Shaheen F, Wang J B, Dufendach K A, Holmes M C, Collman R G, Payne A S, Riley J L. Supraphysiologic control over HIV-1 replication mediated by CD8 T cells expressing a re-engineered CD4-based chimeric antigen receptor. Plos Pathogens. 2017;13(10). doi: ARTN e1006613 10.1371/journal.ppat.1006613. PubMed PMID: WOS:000414163300006.
4. Deeks S G, Wagner B, Anton P A, Mitsuyasu R T, Scadden D T, Haung C, Macken C, Richman D D, Christopherson C, June C H, Lazar R, Broad D F, Jalali S, Hege K M. A phase II randomized study of HIV-specific T-cell gene therapy in subjects with undetectable plasma viremia on combination antiretroviral therapy. Molecular Therapy. 2002; 5(6):788-97. doi: 10.1006/mthe.2002.0611. PubMed PMID: WOS:000176082700020.
5. Lam S, Bollard C. T-cell therapies for HIV. Immunotherapy-Uk. 2013; 5(4):407-14. doi: 10.2217/Imt.13.23. PubMed PMID: WOS:000317055200013.
6. Patel S, Jones R B, Nixon D F, Bollard CM. T-cell therapies for HIV: Preclinical successes and current clinical strategies. Cytotherapy. 2016; 18(8):931-42. Epub 2016/06/07. doi: 10.1016/j.jcyt.2016.04.007. PubMed PMID: 27265874; PMCID: PMC4935558.
7. Long A H, Haso W M, Shern J F, Wanhainen K M, Murgai M, Ingaramo M, Smith J P, Walker A J, Kohler M E, Venkateshwara V R, Kaplan R N, Patterson G H, Fry T J, Orentas R J, Mackall C L. 4-1BB costimulation ameliorates T cell exhaustion induced by tonic signaling of chimeric antigen receptors. Nature medicine. 2015; 21(6):581-90. doi: 10.1038/nm.3838. PubMed PMID: 25939063; PMCID: 4458184.
8. Ghosh A, Smith M, James S E, Davila M L, Velardi E, Argyropoulos K V, Gunset G, Perna F, Kreines F M, Levy E R, Lieberman S, Jay H V, Tuckett A Z, Zakrzewski J L, Tan L, Young L F, Takvorian K, Dudakov J A, Jenq R R, Hanash A M, Motta A C, Murphy G F, Liu C, Schietinger A, Sadelain M, van den Brink M R. Donor CD19 CAR T cells exert potent graft-versus-lymphoma activity with diminished graft-versus-host activity. Nat Med. 2017; 23(2):242-9. doi: 10.1038/nm.4258. PubMed PMID: 28067900.
9. Yang Y, Kohler M E, Chien C D, Sauter C T, Jacoby E, Yan C, Hu Y, Wanhainen K, Qin H, Fry T J. TCR engagement negatively affects CD8 but not CD4 CAR T cell expansion and leukemic clearance. Sci Transl Med. 2017;9(417). doi: 10.1126/scitranslmed.aagl209. PubMed PMID: 29167392.
10. Zheng W, O'Hear C E, Alli R, Basham J H, Abdelsamed H A, Palmer L E, Jones L L, Youngblood B, Geiger T L. PI3K orchestration of the in vivo persistence of chimeric antigen receptor-modified T cells. Leukemia. 2018; 32(5):1157-67. doi: 10.1038/s41375-017-0008-6. PubMed PMID: 29479065; PMCID: PMC5943191.
11. Maude S L, Laetsch T W, Buechner J, Rives S, Boyer M, Bittencourt H, Bader P, Verneris M R, Stefanski H E, Myers G D, Qayed M, De Moerloose B, Hiramatsu H, Schlis K, Davis K L, Martin P L, Nemecek E R, Yanik G A, Peters C, Baruchel A, Boissel N, Mechinaud F, Balduzzi A, Krueger J, June C H, Levine B L, Wood P, Taran T, Leung M, Mueller K T, Zhang Y, Sen K, Lebwohl D, Pulsipher M A, Grupp S A. Tisagenlecleucel in Children and Young Adults with B-Cell Lymphoblastic Leukemia. The New England journal of medicine. 2018; 378(5):439-48. doi: 10.1056/NEJMoa1709866. PubMed PMID: 29385370.
12. Fraietta J A, Lacey S F, Orlando E J, Pruteanu-Malinici I, Gohil M, Lundh S, Boesteanu A C, Wang Y, O'Connor R S, Hwang W T, Pequignot E, Ambrose D E, Zhang C, Wilcox N, Bedoya F, Dorfmeier C, Chen F, Tian L, Parakandi H, Gupta M, Young R M, Johnson F B, Kulikovskaya I, Liu L, Xu J, Kassim S H, Davis M M, Levine B L, Frey N V, Siegel D L, Huang A C, Wherry E J, Bitter H, Brogdon J L, Porter D L, June C H, Melenhorst J J. Determinants of response and resistance to CD19 chimeric antigen receptor (CAR) T cell therapy of chronic lymphocytic leukemia. Nat Med. 2018; 24(5):563-71. Epub 2018/05/02. doi: 10.1038/s41591-018-0010-1. PubMed PMID: 29713085; PMCID: PMC6117613.
13. Ahmed N, Brawley V S, Hegde M, Robertson C, Ghazi A, Gerken C, Liu E, Dakhova O, Ashoori A, Corder A, Gray T, Wu MF, Liu H, Hicks J, Rainusso N, Dotti G, Mei Z, Grilley B, Gee A, Rooney C M, Brenner M K, Heslop H E, Wels W S, Wang L L, Anderson P, Gottschalk S. Human Epidermal Growth Factor Receptor 2 (HER2)-Specific Chimeric Antigen Receptor-Modified T Cells for the Immunotherapy of HER2-Positive Sarcoma. Journal of clinical oncology: official journal of the American Society of Clinical Oncology. 2015; 33(15):1688-96. doi: 10.1200/JCO.2014.58.0225. PubMed PMID: 25800760; PMCID: 4429176.
14. Beatty G L, O'Hara M H, Lacey S F, Torigian D A, Nazimuddin F, Chen F, Kulikovskaya I M, Soulen M C, McGarvey M, Nelson A M, Gladney W L, Levine B L, Melenhorst J J, Plesa G, June C H. Activity of Mesothelin-specific Chimeric Antigen Receptor T cells Against Pancreatic Carcinoma Metastases in a Phase 1 Trial. Gastroenterology. 2018. doi: 10.1053/j.gastro.2018.03.029. PubMed PMID: 29567081.
15. Thistlethwaite F C, Gilham D E, Guest R D, Rothwell D G, Pillai M, Burt D J, Byatte A J, Kirillova N, Valle J W, Sharma S K, Chester K A, Westwood N B, Halford S E R, Nabarro S, Wan S, Austin E, Hawkins RE. The clinical efficacy of first-generation carcinoembryonic antigen (CEACAM5)-specific CAR T cells is limited by poor persistence and transient pre-conditioning-dependent respiratory toxicity. Cancer immunology, immunotherapy: CII. 2017; 66(11):1425-36. Epub 2017/07/01. doi: 10.1007/s00262-017-2034-7. PubMed PMID: 28660319; PMCID: PMC5645435.
16. Heczey A, Louis C U, Savoldo B, Dakhova 0, Durett A, Grilley B, Liu H, Wu MF, Mei Z, Gee A, Mehta B, Zhang H, Mahmood N, Tashiro H, Heslop HE, Dotti G, Rooney CM, Brenner MK. CAR T Cells Administered in Combination with Lymphodepletion and PD-1 Inhibition to Patients with Neuroblastoma. Molecular therapy: the journal of the American Society of Gene Therapy. 2017; 25(9):2214-24. doi: 10.1016/j.ymthe.2017.05.012. PubMed PMID: 28602436; PMCID: PMC5589058.
17. O'Rourke D M, Nasrallah M P, Desai A, Melenhorst J J, Mansfield K, Morrissette J J D, Martinez-Lage M, Brem S, Maloney E, Shen A, Isaacs R, Mohan S, Plesa G, Lacey S F, Navenot J M, Zheng Z, Levine B L, Okada H, June C H, Brogdon J L, Maus M V. A single dose of peripherally infused EGFRvIII-directed CAR T cells mediates antigen loss and induces adaptive resistance in patients with recurrent glioblastoma. Science translational medicine. 2017;9(399). doi: 10.1126/scitranslmed.aaa0984. PubMed PMID: 28724573.
18. Brown C E, Alizadeh D, Starr R, Weng L, Wagner J R, Naranjo A, Ostberg J R, Blanchard M S, Kilpatrick J, Simpson J, Kurien A, Priceman S J, Wang X, Harshbarger T L, D′Apuzzo M, Ressler J A, Jensen M C, Barish M E, Chen M, Portnow J, Forman S J, Badie B. Regression of Glioblastoma after Chimeric Antigen Receptor T-Cell Therapy. The New England journal of medicine. 2016; 375(26):2561-9. doi: 10.1056/NEJMoa1610497. PubMed PMID: 28029927.
19. Maude S L, Frey N, Shaw P A, Aplenc R, Barrett D M, Bunin N J, Chew A, Gonzalez V E, Zheng Z, Lacey S F, Mahnke Y D, Melenhorst J J, Rheingold S R, Shen A, Teachey D T, Levine B L, June C H, Porter D L, Grupp S A. Chimeric antigen receptor T cells for sustained remissions in leukemia. NEnglJMed. 2014; 371(16):1507-17. doi: 10.1056/NEJMoa1407222 [doi].
20. Pauken K E, Sammons M A, Odorizzi P M, Manne S, Godec J, Khan O, Drake A M, Chen Z, Sen D R, Kurachi M, Barnitz R A, Bartman C, Bengsch B, Huang A C, Schenkel J M, Vahedi G, Haining W N, Berger S L, Wherry E J. Epigenetic stability of exhausted T cells limits durability of reinvigoration by PD-1 blockade. Science. 2016; 354(6316):1160-5. Epub 2016/10/30. doi: 10.1 126/science.aaf2807. PubMed PMID: 27789795.
21. Sen D R, Kaminski J, Barnitz R A, Kurachi M, Gerdemann U, Yates K B, Tsao H W, Godec J, LaFleur M W, Brown F D, Tonnerre P, Chung RT, Tully D C, Allen T M, Frahm N, Lauer G M, Wherry E J, Yosef N, Haining W N. The epigenetic landscape of T cell exhaustion. Science. 2016; 354(6316):1165-9. doi: 10.1126/science.aae0491. PubMed PMID: 27789799.
22. Ghoneim H E, Fan Y, Moustaki A, Abdelsamed H A, Dash P, Dogra P, Carter R, Awad W, Neale G, Thomas P G, Youngblood B. De Novo Epigenetic Programs Inhibit PD-1 Blockade-Mediated T Cell Rejuvenation. Cell. 2017; 170(1):142-57 e19. doi: 10.1016/j.cell.2017.06.007. PubMed PMID: 28648661; PMCID: PMC5568784.
23. Youngblood B, Noto A, Porichis F, Akondy R S, Ndhlovu Z M, Austin J W, Bordi R, Procopio F A, Miura T, Allen T M, Sidney J, Sette A, Walker B D, Ahmed R, Boss J M, Sekaly R P, Kaufmann D E. Cutting edge: Prolonged exposure to HIV reinforces a poised epigenetic program for PD-1 expression in virus-specific CD8 T cells. J Immunol. 2013; 191(2):540-4. Epub 2013/06/19. doi: 10.4049/jimmunol.1203161. PubMed PMID: 23772031; PMCID: 3702641.
24. Youngblood B, Oestreich K J, Ha S J, Duraiswamy J, Akondy R S, West E E, Wei Z, Lu P, Austin J W, Riley J L, Boss J M, Ahmed R. Chronic virus infection enforces demethylation of the locus that encodes PD-1 in antigen-specific CD8(+) T cells. Immunity. 2011; 35(3):400-12. Epub 2011/09/29. doi: 10.1016/j.immuni.2011.06.015. PubMed PMID: 21943489; PMCID: 3183460.
25. Nakayama-Hosoya K, Ishida T, Youngblood B, Nakamura H, Hosoya N, Koga M, Koibuchi T, Iwamoto A, Kawana-Tachikawa A. Epigenetic Repression of Interleukin 2 Expression in Senescent CD4+ T Cells During Chronic HIV Type 1 Infection. J Infect Dis. 2014. doi: 10.1093/infdis/jiu376. PubMed PMID: 25001463.
26. Scharer C D, Barwick B G, Youngblood B A, Ahmed R, Boss J M. Global DNA Methylation Remodeling Accompanies CD8 T Cell Effector Function. Journal of immunology. 2013. doi: 10.4049/jimmunol.1301395. PubMed PMID: 23956425.
27. Youngblood B, Oestreich K J, Ha S J, Duraiswamy J, Akondy R S, West E E, Wei Z, Lu P, Austin J W, Riley J L, Boss J M, Ahmed R. Chronic virus infection enforces demethylation of the locus that encodes PD-1 in antigen-specific CD8+ T cells Immunity. 2011;35(3):13.
28. Youngblood B, Wherry E J, Ahmed R. Acquired transcriptional programming in functional and exhausted virus-specific CD8 T cells. Curr Opin HIV AIDS. 2012; 7(1):50-7. Epub 2011/12/03. doi: 10.1097/COH.0b013e32834ddcf2. PubMed PMID: 22134341; PMCID: 3306172.
29. Abdelsamed H A, Moustaki A, Fan Y P, Dogra P, Ghoneim H E, Zebley C C, Triplett B M, Sekaly R P, Youngblood B. Human memory CD8 T cell effector potential is epigenetically preserved during in vivo homeostasis. Journal of Experimental Medicine. 2017; 214(6):1593-606. doi: 10.1084/jem.20161760. PubMed PMID: WOS:000402863300005.
30. Ahn E, Youngblood B, Lee J, Lee J, Sarkar S, Ahmed R. Demethylation of the PD-1 Promoter Is Imprinted during the Effector Phase of CD8 T Cell Exhaustion. J Virol. 2016; 90(19):8934-46. doi: 10.1128/JVI.00798-16. PubMed PMID: 27466420; PMCID: 5021389.
31. Zebley C C, Gottschalk S, Youngblood B. Rewriting History: Epigenetic Reprogramming of CD8(+) T Cell Differentiation to Enhance Immunotherapy. Trends in immunology. 2020; 41(8):665-75. Epub 2020/07/07. doi: 10.1016/j.it.2020.06.008. PubMed PMID: 32624330; PMCID: PMC7395868.
32. Liao J, Karnik R, Gu H, Ziller MJ, Clement K, Tsankov A M, Akopian V, Gifford C A, Donaghey J, Galonska C, Pop R, Reyon D, Tsai S Q, Mallard W, Joung J K, Rinn J L, Gnirke A, Meissner A. Targeted disruption of DNMT1, DNMT3A and DNMT3B in human embryonic stem cells. Nat Genet. 2015; 47(5):469-78. Epub 2015/03/31. doi: 10.1038/ng.3258. PubMed PMID: 25822089; PMCID: PMC4414868.
33. Krenciute G, Prinzing B L, Yi Z, Wu M F, Liu H, Dotti G, Balyasnikova I V, Gottschalk S. Transgenic Expression of IL15 Improves Antiglioma Activity of IL13Ralpha2-CAR T Cells but Results in Antigen Loss Variants. Cancer immunology research. 2017; 5(7):571-81. doi: 10.1158/2326-6066.CIR-16-0376. PubMed PMID: 28550091; PMCID: PMC5746871.
34. Mata M, Gerken C, Nguyen P, Krenciute G, Spencer D M, Gottschalk S. Inducible Activation of MyD88 and CD40 in CAR T Cells Results in Controllable and Potent Antitumor Activity in Preclinical Solid Tumor Models. Cancer discovery. 2017; 7(11):1306-19. doi: 10.1158/2159-8290.CD-17-0263. PubMed PMID: 28801306; PMCID: PMC5780189.
35. Ahmed N, Ratnayake M, Savoldo B, Perlaky L, Dotti G, Wels W S, Bhattacharjee M B, Gilbertson R J, Shine H D, Weiss H L, Rooney C M, Heslop H E, Gottschalk S. Regression of experimental medulloblastoma following transfer of HER2-specific T cells. Cancer Res. 2007; 67(12):5957-64.
36. Krenciute G, Krebs S, Torres D, Wu M F, Liu H, Dotti G, Li X N, Lesniak M S, Balyasnikova I V, Gottschalk S. Characterization and Functional Analysis of scFv-based Chimeric Antigen Receptors to Redirect T Cells to IL13Ralpha2-positive Glioma. Molecular therapy: the journal of the American Society of Gene Therapy. 2016; 24(2):354-63. doi: 10.1038/mt.2015.199. PubMed PMID: 26514825; PMCID: PMC4817815.
37. Yi Z, Prinzing B L, Cao F, Gottschalk S, Krenciute G. Optimizing EphA2-CAR T Cells for the Adoptive Immunotherapy of Glioma. Mol Ther Methods Clin Dev. 2018; 9:70-80. doi: 10.1016/j.omtm.2018.01.009. PubMed PMID: 29552579; PMCID: PMC5852415.
38. Roychoudhuri R, Clever D, Li P, Wakabayashi Y, Quinn K M, Klebanoff C A, Ji Y, Sukumar M, Eil RL, Yu Z, Spolski R, Palmer D C, Pan J H, Patel S J, Macallan D C, Fabozzi G, Shih H Y, Kanno Y, Muto A, Zhu J, Gattinoni L, O'Shea J J, Okkenhaug K, Igarashi K, Leonard W J, Restifo NP. BACH2 regulates CD8(+) T cell differentiation by controlling access of AP-1 factors to enhancers. Nat Immunol. 2016; 17(7):851-60. Epub 2016/05/10. doi: 10.1038/ni.3441. PubMed PMID: 27158840; PMCID: PMC4918801.
39. Kaech S M, Cui W. Transcriptional control of effector and memory CD8+ T cell differentiation. Nat Rev Immunol. 2012; 12(11):749-61. Epub 2012/10/20. doi: 10.1038/nri3307. PubMed PMID: 23080391; PMCID: PMC4137483.
40. Chen Y, Zander R, Khatun A, Schauder D M, Cui W. Transcriptional and Epigenetic Regulation of Effector and Memory CD8 T Cell Differentiation. Front Immunol. 2018; 9:2826. Epub 2018/12/26. doi: 10.3389/fimmu.2018.02826. PubMed PMID: 30581433; PMCID: PMC6292868.
41. Alfei F, Kanev K, Hofmann M, Wu M, Ghoneim H E, Roelli P, Utzschneider D T, von Hoesslin M, Cullen J G, Fan Y, Eisenberg V, Wohlleber D, Steiger K, Merkler D, Delorenzi M, Knolle P A, Cohen C J, Thimme R, Youngblood B, Zehn D. TOX reinforces the phenotype and longevity of exhausted T cells in chronic viral infection. Nature. 2019; 571(7764):265-9. Epub 2019/06/18. doi: 10.1038/s41586-019-1326-9. PubMed PMID: 31207605.
42. Khan O, Giles J R, McDonald S, Manne S, Ngiow S F, Patel K P, Werner M T, Huang A C, Alexander K A, Wu J E, Attanasio J, Yan P, George S M, Bengsch B, Staupe R P, Donahue G, Xu W, Amaravadi R K, Xu X, Karakousis G C, Mitchell T C, Schuchter L M, Kaye J, Berger SL, Wherry E J. TOX transcriptionally and epigenetically programs CD8(+) T cell exhaustion. Nature. 2019; 571(7764):211-8. Epub 2019/06/18. doi: 10.1038/s41586-019-1325-x. PubMed PMID: 31207603; PMCID: PMC6713202.
43. Scott A C, Dundar F, Zumbo P, Chandran S S, Klebanoff C A, Shakiba M, Trivedi P, Menocal L, Appleby H, Camara S, Zamarin D, Walther T, Snyder A, Femia M R, Comen E A, Wen H Y, Hellmann M D, Anandasabapathy N, Liu Y, Altorki N K, Lauer P, Levy O, Glickman M S, Kaye J, Betel D, Philip M, Schietinger A. TOX is a critical regulator of tumour-specific T cell differentiation. Nature. 2019; 571(7764):270-4. Epub 2019/06/18. doi: 10.1038/s41586-019-1324-y. PubMed PMID: 31207604.
44. Abdelsamed H A, Zebley C C, Nguyen H, Rutishauser R L, Fan Y, Ghoneim H E, Crawford J C, Alfei F, Alli S, Ribeiro S P, Castellaw A H, McGargill M A, Jin H, Boi S K, Speake C, Serti E, Turka L A, Busch M E, Stone M, Deeks S G, Sekaly R P, Zehn D, James E A, Nepom GT, Youngblood B. Beta cell-specific CD8(+) T cells maintain stem cell memory-associated epigenetic programs during type 1 diabetes. Nature immunology. 2020. Epub 2020/04/02. doi: 10.1038/s41590-020-0633-5. PubMed PMID: 32231298.
45. Schultz L, Mackall C. Driving CAR T cell translation forward. Science translational medicine. 2019;11(481). Epub 2019/03/01. doi: 10.1126/scitranslmed.aaw2127. PubMed PMID: 30814337.
46. Feucht J, Sun J, Eyquem J, Ho Y J, Zhao Z, Leibold J, Dobrin A, Cabriolu A, Hamieh M, Sadelain M. Calibration of CAR activation potential directs alternative T cell fates and therapeutic potency. Nature medicine. 2019; 25(1):82-8. Epub 2018/12/19. doi: 10.1038/s41591-018-0290-5. PubMed PMID: 30559421; PMCID: PMC6532069.
47. Sommermeyer D, Hudecek M, Kosasih P L, Gogishvili T, Maloney D G, Turtle C J, Riddell S R. Chimeric antigen receptor-modified T cells derived from defined CD8+ and CD4+ subsets confer superior antitumor reactivity in vivo. Leukemia. 2016; 30(2):492-500. doi: 10.1038/leu.2015.247. PubMed PMID: 26369987; PMCID: PMC4746098.
48. Gattinoni L, Lugli E, Ji Y, Pos Z, Paulos C M, Quigley M F, Almeida J R, Gostick E, Yu Z, Carpenito C, Wang E, Douek D C, Price D A, June C H, Marincola F M, Roederer M, Restifo N P. A human memory T cell subset with stem cell-like properties. NatMed. 2011; 17(10):1290-7. doi: nm.2446 [pii];10.1038/nm.2446 [doi].
49. Krishna S, Lowery F J, Copeland A R, Bahadiroglu E, Mukherjee R, Jia L, Anibal J T, Sachs A, Adebola SO, Gurusamy D, Yu Z, Hill V, Gartner J J, Li Y F, Parkhurst M, Paria B, Kvistborg P, Kelly M C, Goff S L, Altan-Bonnet G, Robbins P F, Rosenberg S A. Stem-like CD8 T cells mediate response of adoptive cell immunotherapy against human cancer. Science. 2020; 370(6522):1328-34. Epub 2020/12/12. doi: 10.1126/science.abb9847. PubMed PMID: 33303615.
50. Park J H, Riviere I, Gonen M, Wang X, Senechal B, Curran K J, Sauter C, Wang Y, Santomasso B, Mead E, Roshal M, Maslak P, Davila M, Brentjens R J, Sadelain M. Long-Term Follow-up of CD19 CAR Therapy in Acute Lymphoblastic Leukemia. The New England journal of medicine. 2018; 378(5):449-59. doi: 10.1056/NEJMoa1709919. PubMed PMID: 29385376.
51. Sade-Feldman M, Yizhak K, Bjorgaard S L, Ray J P, de Boer C G, Jenkins R W, Lieb D J, Chen J H, Frederick D T, Barzily-Rokni M, Freeman S S, Reuben A, Hoover P J, Villani A C, Ivanova E, Portell A, Lizotte P H, Aref A R, Eliane J P, Hammond M R, Vitzthum H, Blackmon S M, Li B, Gopalakrishnan V, Reddy S M, Cooper Z A, Paweletz C P, Barbie D A, Stemmer-Rachamimov A, Flaherty K T, Wargo J A, Boland G M, Sullivan R J, Getz G, Hacohen N. Defining T Cell States Associated with Response to Checkpoint Immunotherapy in Melanoma. Cell. 2018; 175(4):998-1013 e20. Epub 2018/11/06. doi: 10.1016/j.cell.2018.10.038. PubMed PMID: 30388456.
52. Lynn R C, Weber E W, Sotillo E, Gennert D, Xu P, Good Z, Anbunathan H, Lattin J, Jones R, Tieu V, Nagaraja S, Granja J, de Bourcy C F A, Majzner R, Satpathy A T, Quake S R, Monje M, Chang H Y, Mackall C L. c-Jun overexpression in CAR T cells induces exhaustion resistance. Nature. 2019; 576(7786):293-300. Epub 2019/12/06. doi: 10.1038/s41586-019-1805-z. PubMed PMID: 31802004.
53. Moskophidis D, Lechner F, Pircher H, Zinkernagel R M. Virus persistence in acutely infected immunocompetent mice by exhaustion of antiviral cytotoxic effector T cells. Nature. 1993; 362(6422):758-61. Epub 1993/04/22. doi: 10.1038/362758a0. PubMed PMID: 8469287.
54. Zajac A J, Blattman J N, Murali-Krishna K, Sourdive D J, Suresh M, Altman J D, Ahmed R. Viral immune evasion due to persistence of activated T cells without effector function. J Exp Med. 1998; 188(12):2205-13. Epub 1998/12/22. doi: 10.1084/jem.188.12.2205. PubMed PMID: 9858507; PMCID: PMC2212420.
55. Hui E, Cheung J, Zhu J, Su X, Taylor M J, Wallweber H A, Sasmal D K, Huang J, Kim J M, Mellman I, Vale R D. T cell costimulatory receptor CD28 is a primary target for PD-1-mediated inhibition. Science. 2017; 355(6332):1428-33. Epub 2017/03/11. doi: 10.1126/science.aafl292. PubMed PMID: 28280247; PMCID: PMC6286077.
56. Kamphorst A O, Wieland A, Nasti T, Yang S, Zhang R, Barber D L, Konieczny B T, Daugherty C Z, Koenig L, Yu K, Sica G L, Sharpe A H, Freeman G J, Blazar B R, Turka L A, Owonikoko T K, Pillai R N, Ramalingam S S, Araki K, Ahmed R. Rescue of exhausted CD8 T cells by PD-1-targeted therapies is CD28-dependent. Science. 2017; 355(6332):1423-7. Epub 2017/03/11. doi: 10.1126/science.aaf0683. PubMed PMID: 28280249; PMCID: PMC5595217.
57. Brooks D G, Trifilo M J, Edelmann K H, Teyton L, McGavern D B, Oldstone MB. Interleukin-10 determines viral clearance or persistence in vivo. Nature medicine. 2006; 12(11):1301-9. Epub 2006/10/17. doi: 10.1038/nm1492. PubMed PMID: 17041596; PMCID: PMC2535582.
58. Ley T J, Ding L, Walter M J, McLellan M D, Lamprecht T, Larson D E, Kandoth C, Payton J E, Baty J, Welch J, Harris C C, Lichti C F, Townsend R R, Fulton R S, Dooling D J, Koboldt D C, Schmidt H, Zhang Q, Osborne J R, Lin L, O'Laughlin M, McMichael J F, Delehaunty K D, McGrath S D, Fulton L A, Magrini V J, Vickery T L, Hundal J, Cook L L, Conyers J J, Swift G W, Reed J P, Alldredge P A, Wylie T, Walker J, Kalicki J, Watson M A, Heath S, Shannon W D, Varghese N, Nagarajan R, Westervelt P, Tomasson M H, Link D C, Graubert T A, DiPersio J F, Mardis E R, Wilson R K. DNMT3A mutations in acute myeloid leukemia. The New England journal of medicine. 2010; 363(25):2424-33. Epub 2010/11/12. doi: 10.1056/NEJMoa1005143. PubMed PMID: 21067377; PMCID: PMC3201818.
59. Walter M J, Ding L, Shen D, Shao J, Grillot M, McLellan M, Fulton R, Schmidt H, Kalicki-Veizer J, O'Laughlin M, Kandoth C, Baty J, Westervelt P, DiPersio J F, Mardis E R, Wilson R K, Ley T J, Graubert T A. Recurrent DNMT3A mutations in patients with myelodysplastic syndromes. Leukemia. 2011; 25(7):1153-8. Epub 2011/03/19. doi: 10.1038/leu.2011.44. PubMed PMID: 21415852; PMCID: PMC3202965.
60. Youngblood B, Hale J S, Kissick H T, Ahn E, Xu X, Wieland A, Araki K, West E E, Ghoneim H E, Fan Y, Dogra P, Davis C W, Konieczny B T, Antia R, Cheng X, Ahmed R. Effector CD8 T cells dedifferentiate into long-lived memory cells. Nature. 2017; 552(7685):404-9. doi: 10.1038/nature25144. PubMed PMID: 29236683; PMCID: PMC5965677.
61. Kramer A C, Kothari A, Wilson W C, Celik H, Nikitas J, Mallaney C, Ostrander E L, Eultgen E, Martens A, Valentine M C, Young A L, Druley T E, Figueroa M E, Zhang B, Challen G A. Dnmt3a regulates T-cell development and suppresses T-ALL transformation. Leukemia. 2017; 31(11):2479-90. Epub 2017/03/23. doi: 10.1038/leu.2017.89. PubMed PMID: 28321121; PMCID: PMC5636646.
62. Fraietta J A, Nobles C L, Sammons M A, Lundh S, Carty S A, Reich T J, Cogdill A P, Morrissette J J D, DeNizio J E, Reddy S, Hwang Y, Gohil M, Kulikovskaya I, Nazimuddin F, Gupta M, Chen F, Everett J K, Alexander K A, Lin-Shiao E, Gee M H, Liu X, Young R M, Ambrose D, Wang Y, Xu J, Jordan M S, Marcucci K T, Levine B L, Garcia K C, Zhao Y, Kalos M, Porter D L, Kohli R M, Lacey S F, Berger S L, Bushman F D, June C H, Melenhorst J J. Disruption of TET2 promotes the therapeutic efficacy of CD19-targeted T cells. Nature. 2018; 558(7709):307-12. doi: 10.1038/s41586-018-0178-z. PubMed PMID: 29849141.
63. Chow K K, Naik S, Kakarla S, Brawley V S, Shaffer D R, Yi Z, Rainusso N, Wu M F, Liu H, Kew Y, Grossman R G, Powell S, Lee D, Ahmed N, Gottschalk S. T Cells Redirected to EphA2 for the Immunotherapy of Glioblastoma. MolTher. 2013; 21(3):629-37. doi: mt2012210 [pii];10.1038/mt.2012.210 [doi].
64. Ahmed N, Salsman V S, Yvon E, Louis C U, Perlaky L, Wels W S, Dishop M K, Kleinerman E E, Pule M, Rooney C M, Heslop H E, Gottschalk S. Immunotherapy for osteosarcoma: genetic modification of T cells overcomes low levels of tumor antigen expression. MolTher. 2009; 17(10):1779-87.
65. Li W, Guo L, Rathi P, Marinova E, Gao X, Wu M F, Liu H, Dotti G, Gottschalk S, Metelitsa L S, Heczey A. Redirecting T Cells to Glypican-3 with 4-1BB Zeta Chimeric Antigen Receptors Results in Th1 Polarization and Potent Antitumor Activity. Human gene therapy. 2017;28(5):437-48. doi: 10.1089/hum.2016.025. PubMed PMID: 27530312; PMCID: PMC5444493.
66. Connelly J P, Pruett-Miller S M. CRIS.py: A Versatile and High-throughput Analysis Program for CRISPR-based Genome Editing. Scientific reports. 2019;9(1):4194. Epub 2019/03/14. doi: 10.1038/s41598-019-40896-w. PubMed PMID: 30862905; PMCID: PMC6414496.
67. Abdelsamed H A, Moustaki A, Fan Y, Dogra P, Ghoneim H E, Zebley C C, Triplett B M, Sekaly R P, Youngblood B. Human memory CD8 T cell effector potential is epigenetically preserved during in vivo homeostasis. J Exp Med. 2017; 214(6):1593-606. doi: 10.1084/jem.20161760. PubMed PMID: 28490440; PMCID: PMC5461005.
68. Xi Y, Li W. BSMAP: whole genome bisulfite sequence MAPping program. BMC Bioinformatics. 2009; 10:232. Epub 2009/07/29. doi: 10.1186/1471-2105-10-232. PubMed PMID: 19635165; PMCID: PMC2724425.
69. Sun D, Xi Y, Rodriguez B, Park H J, Tong P, Meong M, Goodell M A, Li W. MOABS: model based analysis of bisulfite sequencing data. Genome biology. 2014;15(2):R38. doi: 10.1186/gb-2014-15-2-r38. PubMed PMID: 24565500; PMCID: 4054608.
70. Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: a knowledge-based approach for interpreting genome-wide expression profiles. Proceedings of the National Academy of Sciences of the United States of America. 2005; 102(43):15545-50. Epub 2005/10/04. doi: 10.1073/pnas.0506580102. PubMed PMID: 16199517; PMCID: PMC1239896.
71. Fonseca R, Beura L K, Quarnstrom C F, Ghoneim H E, Fan Y, Zebley C C, Scott M C, Fares-Frederickson N J, Wijeyesinghe S, Thompson E A, Borges da Silva H, Vezys V, Youngblood B, Masopust D. Developmental plasticity allows outside—in immune responses by resident memory T cells. Nature immunology. 2020; 21(4):412-21. Epub 2020/02/19. doi: 10.1038/s41590-020-0607-7. PubMed PMID: 32066954; PMCID: PMC7096285.
72. Malta T M, Sokolov A, Gentles A J, Burzykowski T, Poisson L, Weinstein I N, Kaminska B, Huelsken J, Omberg L, Gevaert O, Colaprico A, Czerwinska P, Mazurek S, Mishra L, Heyn H, Krasnitz A, Godwin A K, Lazar A J, Cancer Genome Atlas Research N, Stuart J M, Hoadley K A, Laird P W, Noushmehr H, Wiznerowicz M. Machine Learning Identifies Stemness Features Associated with Oncogenic Dedifferentiation. Cell. 2018; 173(2):338-54 e15. Epub 2018/04/07. doi: 10.1016/j.cell.2018.03.034. PubMed PMID: 29625051; PMCID: PMC5902191.
73. Sokolov A, Carlin D E, Paull E O, Baertsch R, Stuart J M. Pathway-Based Genomics Prediction using Generalized Elastic Net. PLoS Comput Biol. 2016;12(3):e1004790. Epub 2016/03/10. doi: 10.1371/journal.pcbi.1004790. PubMed PMID: 26960204; PMCID: PMC4784899.
74. Robinson M D, McCarthy D J, Smyth G K. edgeR: a Bioconductor package for differential expression analysis of digital gene expression data. Bioinformatics. 2010; 26(1):139-40. Epub 2009/11/17. doi: 10.1093/bioinformatics/btp616. PubMed PMID: 19910308; PMCID: PMC2796818.
75. Ritchie M E, Phipson B, Wu D, Hu Y, Law CW, Shi W, Smyth G K. limma powers differential expression analyses for RNA-sequencing and microarray studies. Nucleic Acids Res. 2015;43(7):e47. Epub 2015/01/22. doi: 10.1093/nar/gkv007. PubMed PMID: 25605792; PMCID: PMC4402510.
76. Wickham H. ggplot2: elegant graphics for data analysis: Springer; 2016.
77. Yugang Guo, Yu-Qing Xie, Min Gao, Yang Zhao, Fabien Franco, Mathias Wenes, Imran Siddiqui, Alessio Bevilacqua, Haiping Wang, Hanshuo Yang, Bing Feng, Xin Xie, Catherine M Sabatel, Benjamin Tschumi, Amphun Chaiboonchoe, Yuxi Wang, Weimin Li, Weihua Xiao, Werner Held, Pedro Romero, Ping-Chih Ho, Li Tang. Metabolic reprogramming of terminally exhausted CD8+ T cells by IL-10 enhances anti-tumor immunity. Nat Immunol. 2021 Jun;22(6):746-756. doi: 10.1038/s41590-021-00940-2. PMID: 34031618.
78. Wenjun Ouyang & Anne O'Garra. IL-10 Family Cytokines IL-10 and IL-22: from Basic Science to Clinical Translation. Immunity. 2019 Apr. 16; 50(4):871-891. doi: 10.1016/j.immuni.2019.03.020. PMID: 30995504.
79. Xiaoting Wang, Kit Wong, Wenjun Ouyang, Sascha Rutz. Targeting IL-10 Family Cytokines for the Treatment of Human Diseases. Cold Spring Harb Perspect Biol. 2019 Feb. 1; 11(2):a028548. doi: 10.1101/cshperspect.a028548. PMID: 29038121.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

1. A modified immune effector cell, wherein (i) DNA (cytosine-5)-methyltransferase 3A (DNMT3A)-mediated de novo DNA methylation of the cell genome is inhibited, and (ii) IL10 signaling pathway is enhanced.

2. The modified immune effector cell of claim 1, wherein DNMT3A-mediated methylation is inhibited by inhibiting the enzymatic activity of the DNMT3A protein in the cell.

3. The modified immune effector cell of claim 2, wherein the enzymatic activity of the DNMT3A protein is inhibited by exposing the cell to a DNMT3A active site inhibitor.

4. The modified immune effector cell of claim 2, wherein the DNMT3A gene is mutated in a DNMT3A catalytic domain so that the enzymatic activity of the DNMT3A protein is inhibited.

5. The modified immune effector cell of claim 1, wherein DNMT3A-mediated methylation is inhibited by deleting the DNMT3A gene or inhibiting expression of the DNMT3A gene.

6. The modified immune effector cell of claim 1, wherein the level of functional DNMT3A protein in the cell is decreased by about 50%, 70%, 90%, 99% or more.

7-9. (canceled)

10. The modified immune effector cell of claim 1, wherein IL1O signaling pathway in the immune effector cell is enhanced by subjecting the cell to an effective amount of an exogenous IL10 or a carrier comprising the exogenous IL10.

11. The modified immune effector cell of claim 10, wherein the exogenous IL1O is a recombinant IL10.

12. The modified immune effector cell of claim 10, wherein the carrier is a nanoparticle.

13. The modified immune effector cell of claim 1, wherein the immune effector cell has been activated and/or expanded ex vivo.

14. The modified immune effector cell of claim 10, wherein the immune effector cell is subjected to the exogenous IL1O at the beginning of cell expansion.

15. The modified immune effector cell of claim 10, wherein the immune effector cell is subjected to the exogenous IL10 more than once.

16. The modified immune effector cell of claim 1, wherein the IL10 signaling pathway is enhanced by genetically modifying the immune effector cell to express IL10.

17. The modified immune effector cell of claim 16, wherein IL10 is expressed from a transgene encoding IL10 introduced into the immune effector cell or wherein IL10 is constitutively expressed.

18. The modified immune effector cell of claim 16, wherein the transgene encoding IL10 is inserted into the DNMT3A locus.

19-21. (canceled)

22. The modified immune effector cell of claim 1, wherein the immune effector cell is a T cell or a Nature Killer (NK) cell.

23. The modified immune effector cell of claim 22, wherein the T cell is selected from a CD8+ T cell, a CD4+ T cell, a cytotoxic T cell, an αβ T cell receptor (TCR) T cell, a natural killer T (NKT) cell, a γδ T cell, a memory T cell, a T-helper cell, and a regulatory T cell (Treg).

24. (canceled)

25. The modified immune effector cell of claim 1, wherein the cell further comprises at least one surface molecule capable of binding specifically to an antigen.

26-27. (canceled)

28. The modified immune effector cell of claim 1, wherein the cell further comprises a chimeric antigen receptor (CAR), an antigen specific T-cell receptor (TCR), a bispecific antibody, and/or a T cell antigen coupler (TAC).

29. The modified immune effector cell of claim 28, wherein the cell further comprises a chimeric antigen receptor (CAR).

30-54. (canceled)

55. The modified immune effector cell of claim 1, wherein the immune effector cell is an allogeneic cell or an autologous cell.

56. (canceled)

57. The modified immune effector cell of claim 1, wherein the immune effector cell is isolated from a subject having a disease.

58. The modified immune effector cell of claim 57, wherein the disease is a cancer, an infectious disease, an inflammatory disorder, or an autoimmune disease.

59-62. (canceled)

63. A method of preserving multipotency of an immune effector cell, said method comprising (i) deleting or modifying a DNA (cytosine-5)-methyltransferase 3A (DNMT3A) gene or gene product in the cell so that the DNMT3A-mediated de novo DNA methylation of the cell genome is inhibited; and (ii) enhancing IL10 signaling pathway in the immune effector cell.

64-99. (canceled)

100. A pharmaceutical composition comprising the modified immune effector cell of claim 1 and a pharmaceutically acceptable carrier and/or excipient.

101. A method of treating a disease in a subject in need thereof comprising administering to the subject an effective amount of the modified immune effector cell of claim 1 or a pharmaceutical composition comprising said modified immune effector cell.

102-116. (canceled)
